(19) United States
(12) Patent Application Publication
(10) Pub. No.: US 2022/0288057 A1
(43) Pub. Date: Sep. 15, 2022

(54) CONJOINT THERAPY FOR TREATING SEIZURE DISORDERS

(71) Applicant: XENON PHARMACEUTICALS INC., Burnaby (CA)

(72) Inventor: James Philip JOHNSON, JR., Vancouver (CA)

(21) Appl. No.: 17/668,316

(22) Filed: Feb. 9, 2022

Related U.S. Application Data
(60) Provisional application No. 63/147,736, filed on Feb. 9, 2021.

(51) Int. Cl. A61K 31/472; A61K 31/19; A61K 31/4015; A61K 31/4166; A61K 31/165; A61K 31/41; A61P 25/08

(52) U.S. Cl. CPC A61K 31/472; A61K 31/19; A61K 31/4015; A61K 31/4166; A61K 31/165; A61K 31/41; A61P 25/08

(57) ABSTRACT

In certain embodiments, the present disclosure is directed to methods and uses for treating seizure disorders in a human in need thereof, wherein the methods and uses comprise conjointly administering N-[4-(6-fluoro-3,4-dihydro-1H-isoquinolin-2-yl)-2,6-dimethylphenyl]-3,3-dimethylbutanamide (Compound A) and an antiseizure medication (ASM) to the human in amounts that are therapeutically effective when conjointly administered. The present disclosure is further directed to various improved methods of therapy and administration of Compound A.

### 1. BACKGROUND

Epilepsy is a common neurological disorder, with a worldwide estimated prevalence of 0.7% of the population (i.e., about 50 million people) (see Hirtz, D. et al., Neurology, (2007), 68:326-337). It is characterized by abnormal electrical activities in the brain leading to seizures. For epidemiological purposes, the definition requires more than one unprovoked seizure of any type.

Patients with epilepsy have an increased mortality risk compared with the general population due primarily to the etiology of the disease. However, in patients with uncontrolled epilepsy, the greatest seizure-related risk of mortality is due to sudden unexpected death in epilepsy (SUDEP) (see, Hitiris, N. et al., Epilepsy and Behavior (2007), 10:363-376. Patients who participate in clinical trials of investigational antiseizure medications (ASMs) generally have had epilepsy for more than 10 years and have failed multiple ASM therapies.

The pathophysiology of most forms of epilepsy remains poorly understood, but it is known that epileptic seizures arise from an excessively synchronous and sustained firing of a group of neurons. Persistent increase in neuronal excitability is common to all epileptic syndromes. The therapeutic strategy in treating epilepsy involves reducing neuronal excitability through various mechanistic pathways. Over the past two decades, several new ASMs were developed and marketed to expand the therapeutic spectrum by targeting different mechanisms of action and to improve the risk/benefit profile. Currently available ASMs are considered to act by inhibition of synaptic vesicle glycoprotein, potentiation of the inhibitory GABAergic neurotransmission, reduction of glutamate-mediated excitatory neurotransmission, or inhibition of voltage-gated sodium or calcium channels. Despite this, up to 30% of patients remain refractory to conventional treatment and continue to have uncontrolled seizures (see Brown, D. A. et al., Nature (1980), 283:673-676, and Elger, C. E. et al., Epilepsy Behav. (2008), 12:501-539). The quality of life in refractory patients is poor; they cannot drive a car, and they have difficulty working or living independently. Additionally, many patients have behavioral, neurological, and/or intellectual disturbances as sequelae of their seizure disorder. Current agents have minimal to no effects on neuronal potassium-gated channels, in spite of the fact that these channels have a major role in the control of neuronal excitability. Medicines with novel mechanisms of action, or medicines that improve on the already marketed ASMs are therefore needed to address the significant unmet clinical need for seizure control in patients with treatment-resistant epilepsy.

The voltage-gated potassium channels Kv7.2 and Kv7.3 (Kv7.2/Kv7.3) are important in controlling neuronal excitability. Kv7.2/Kv7.3 underlie the neuronal “M-current”, named according to its initial characterization as a neuronal current decreased in response to muscarinic/cholinergic agonists (see Brown, D. A. et al., Nature (1980), 283:673-676). The M-current is a non-inactivating, hyperpolarizing current known to act as a brake on neuronal hyperexcitability. Consequently, a decrease in the Kv7.2-mediated M-current, for example through genetic loss-of-function, can cause neuronal depolarization and an increase in membrane and neuronal excitability that can lead to action potential bursts that manifest as epileptic seizures. In contrast, an increase in the Kv7.2-mediated M-current can hyperpolarize the cell membrane and thereby reduce neuronal excitability and prevent the initiation and propagation of action potential bursts and the resultant seizures. Enhancing the open state of Kv7.2/Kv7.3 channels in neurons favors a hyperpolarized resting state, which reduces rapid action potential spiking (i.e., burst firing). Such enhancement can provide a stabilizing effect on excitable, particularly hyper-excitable, neurons and therefore be useful in treating certain seizure disorders. This enhancement has been clinically proven to be effective for treatment of seizure disorders, such as partial onset seizures in adults with epilepsy, with retigabine (ezogabine), a known Kv7.2/Kv7.3 opener.

While significant advances have been made in this field, particularly in the context of Compound A, defined below, and its use in treating seizure disorders, there remains a substantial need to provide patients further options for treating seizure disorders.

### 2. SUMMARY

The present disclosure describes certain methods and uses for the small molecule N-[4-(6-Fluoro-3,4-dihydro-1H-isoquinolin-2-yl)-2,6-dimethylphenyl]-3,3-dimethylbutanamide (herein referred to as “Compound A”).

In one embodiment, the present disclosure is directed to a method of treating a seizure disorder in a human in need thereof, comprising conjointly administering Compound A and an ASM to the human in amounts that are therapeutically effective when conjointly administered. Likewise, in some embodiments, the present disclosure is directed to the use of Compound A and an ASM, in amounts that are therapeutically effective when conjointly administered, in treating a seizure disorder in a human in need thereof.

In another embodiment, the present disclosure is directed to a method of reducing the amount of an ASM that is required for therapeutic efficacy in a human suffering from a seizure disorder, comprising administering to the human, conjointly with the ASM, an amount of Compound A that is effective to achieve such reduction when administered with the ASM. Likewise, in some embodiments, the present disclosure is directed to the use of Compound A in reducing the amount of an ASM that is required for therapeutic efficacy in a human suffering from a seizure disorder, such as by administering to the human, conjointly with the ASM, an amount of Compound A that is effective to achieve such reduction when administered with the ASM. In certain instances of these embodiments, the ASM is valproic acid, levetiracetam, phenytoin, lacosamide, cenobamate, or a combination thereof, particularly valproic acid.

In one embodiment, the present disclosure is directed to a method of reducing the amount of Compound A that is required for therapeutic efficacy in a human suffering from a seizure disorder, comprising administering to the human, conjointly with Compound A, an amount of the ASM that is effective to achieve such reduction when administered with Compound A. Likewise, in some embodiments, the present disclosure is directed to the use of an ASM in reducing the amount of Compound A that is required for therapeutic efficacy in a human suffering from a seizure disorder, such as by administering to the human, conjointly with Compound A, an amount of the ASM that is effective to achieve such reduction when administered with Compound A. In certain instances of these embodiments, the ASM is valproic acid, levetiracetam, phenytoin, lacosamide, cenobamate, or a combination thereof, particularly phenytoin.

In some aspects, the methods and uses described herein of treating a seizure disorder in a human in need thereof, of reducing the amount of an ASM required for therapeutic efficacy, of reducing the amount of Compound A required for therapeutic efficacy, or of reducing absorption of an administered amount of Compound A into plasma or brain in a human while effectively treating a seizure disorder, comprise enhancing the opening of a Kv7 potassium channel in the human.

In one embodiment, the present disclosure is directed to a method of enhancing the opening of a Kv7 potassium channel in a human, comprising conjointly administering Compound A and an ASM to the human in amounts that are therapeutically effective when conjointly administered, such as wherein the human has a seizure disorder. Likewise, in some embodiments, the present disclosure is directed to the use of Compound A and an ASM, in amounts that are therapeutically effective when conjointly administered, in enhancing the opening of a Kv7 potassium channel in a human, for example, wherein the human has a seizure disorder.

In some aspects, the Kv7 potassium channel is one or more of Kv7.2, Kv7.3, Kv7.4, or Kv7.5. In certain instances, the opening or enhanced opening of one or more of the Kv7.2, Kv7.3, Kv7.4, or Kv7.5 potassium channels is selective over Kv7.1. In other instances, the method comprises opening or enhanced opening of the Kv7.2/Kv7.3 (KCNQ2/3) potassium channel.

In some aspects of the present methods and uses, the ASM is a benzodiazepine, carbamazepine, cenobamate, felbamate, gabapentin, lacosamide, lamotrigine, levetiracetam, oxcarbazepine, phenobarbital, phenytoin, pregabalin, rufinamide, tiagabine, topiramate, valproic acid, vigabatrin, zonisamide, or a combination thereof. In particular aspects, the antiseizure medication is valproic acid, levetiracetam, phenytoin, lacosamide, cenobamate, or a combination thereof. In certain instances, the antiseizure medication does not enhance the opening of a Kv7 potassium channel in the human.

In some embodiments, the ASM decreases neuronal excitation by blocking a sodium channel in the human, decreases neuronal excitation by blocking a calcium channel in the human, decreases neuronal excitation by binding to synaptic vesicle glycoprotein 2A (SV2A) in the human, or increases neuronal inhibition in the human.

In some aspects the ASM is a glutamatergic agent. In other aspects, the ASM is a GABAergic agent.

In some instances, the seizure disorder treated by or associated with the present methods is associated with Kv7 potassium channel dysfunction. In other instances, the seizure disorder is focal onset epilepsy.

In some embodiments of the present methods and uses, Compound A is orally administered to the human. In certain embodiments of the present methods and uses, the ASM is orally administered to the human. In further embodiments of the present methods and uses, both Compound A and the ASM are orally administered to the human.

In some aspects of the present methods and uses, Compound A is administered, e.g., orally, conjointly with an ASM, at a dose of Compound A of 1 to 200 mg to the human, at a dose of Compound A of 2 to 100 mg to the human, at a dose of Compound A of 5 to 50 mg to the human, at a dose of Compound A of 5, 10, 15, 20, or 25 mg to the human, or at a dose of Compound A of 20 mg to the human. In other aspects, Compound A is administered, e.g., orally, conjointly with an ASM, at a dose of Compound A of at least 10 mg to the human, at a dose of Compound A of at least 20 mg to the human, or at a dose of Compound A of at least 50 mg to the human. In other aspects, Compound A is administered, e.g., orally, conjointly with an ASM, at a dose of Compound A of 5-1000 mg per day to the human, at a dose of Compound A of 5-500 mg per day to the human, at a dose of Compound A of 5-250 mg per day to the human, at a dose of Compound A of 20-150 mg per day to the human, or at a dose of Compound A of 100 mg per day to the human. In other instances, Compound A is administered, e.g., orally, conjointly with an ASM, at a dose of Compound A of 0.01-2.0 mg/kg to the human, at a dose of Compound A of 0.03-1.0 mg/kg to the human, or at a dose of Compound A of 0.05-0.5 mg/kg to the human.

In some embodiments of the present methods and uses, Compound A is orally administered to the human from between about 30 minutes before to about 2 hours after eating a meal, for example, Compound A may be orally administered to the human during a meal or within 15 minutes after eating a meal.

In another embodiment of the present methods and uses, the ASM is valproic acid. In some aspects, the valproic acid is administered conjointly with Compound A at a dose of valproic acid of 2-16 mg/kg to the human, for example, valproic acid may be administered at a dose of 4-12 mg/kg to the human.

In another embodiment of the present methods and uses, the ASM is phenytoin. In some aspects, the phenytoin is administered conjointly with Compound A at a dose of phenytoin of 0.05-5 mg/kg to the human, for example, phenytoin may be administered at a dose of 0.1-1 mg/kg to the human.

In another embodiment of the present methods and uses, the ASM is lacosamide. In some aspects, the lacosamide is administered conjointly with Compound A at a dose of lacosamide of 0.1-5 mg/kg to the human, for example, the lacosamide is administered at a dose of 0.5-1 mg/kg to the human.

In another embodiment of the present methods and uses, the ASM is cenobamate. In some aspects, the cenobamate is administered conjointly with Compound A at a dose of cenobamate of 0.05-5 mg/kg to the human, for example, the cenobamate is administered at a dose of 0.1-1 mg/kg to the human.

In certain embodiments of the present methods and uses, the conjoint administration of Compound A and the ASM (e.g., valproic acid, phenytoin, levetiracetam, lacosamide, cenobamate, or a combination thereof) provides improved efficacy (e.g., increases the reduction in the number of seizure episodes or reduction of the severity of seizure episodes in the human) relative to individual administration of Compound A or the ASM alone. In certain such embodiments, the conjoint administration provides an additive effect, wherein an additive effect refers to the sum of the individual effects of administering Compound A and an ASM. In some embodiments, the conjoint administration provides a synergistic effect, wherein a synergistic effect refers to an effect that is greater than the sum of the individual effects of administering Compound A and an ASM.

In other embodiments, the present disclosure provides a pharmaceutical composition comprising Compound A, an antiseizure medication (ASM), and a pharmaceutically acceptable carrier. In some aspects of the pharmaceutical composition, the ASM is a benzodiazepine, carbamazepine, cenobamate, felbamate, gabapentin, lacosamide, lamotrigine, levetiracetam, oxcarbazepine, phenobarbital, phenytoin, pregabalin, rufinamide, tiagabine, topiramate, valproic acid, vigabatrin, zonisamide, or a combination thereof.

Compound A is a small molecule currently being developed for the treatment of seizure disorders, and its use as a potassium channel modulator is disclosed in U.S. Pat. Nos. 8,293,911 and 8,993,593 as well as U.S. application Ser. Nos. 16/409,684 and 16/410,851, the disclosures of which are hereby incorporated by reference in their entireties.

These and other aspects of this disclosure will be apparent upon reference to the following detailed description. To this end, various references are set forth herein which describe in more detail certain background information and procedures and are each hereby incorporated by reference in their entirety.

### 3. BRIEF DESCRIPTION OF THE DRAWINGS

FIG. 1 shows results for the PO administration of Compound A at 1, 2, 4, 8 mg/kg or vehicle 2 hours before the assay in CF-1 mice (n=7 per group). Upper graph: fraction of mice displaying hindlimb tonic extensor component during the MES test. Each bar indicates the mean response±S.E.M. Lower graph: percentage of mice protected from tonic seizure. A single asterisk in this and all other figures indicates 0.01<p<0.05. “Mpk” recited is this and all other figures is mg/kg.

FIG. 2 shows results for the PO administration of Compound A at 1, 2, 4, 8 mg/kg or vehicle 0.5 hours before the assay in CF-1 mice (n=7 per dose group, n=5 for vehicle alone). Upper graph: presence or absence of hindlimb tonic extensor component during the tonic seizure per animal Lower graph: percentage of mice protected.

FIG. 3 shows the pharmacokinetic (PK) and pharmacodynamics (PD) properties of Compound A in the mouse MES assay. The horizontal error bars indicate the S.E.M. of the plasma or brain concentration. When not visible, it is because they are smaller than the symbol indicating the mean value. The solid curve through each set of data collected 2 hours after dosing is the best fit to a concentration-response curve. The IC₅₀ based upon brain and plasma concentrations were 275 nM and 154 nM, respectively. The efficacy measured at 0.5 hours after dosing was consistent with the PK-PD relationship determined 2 hours after dosing.

FIG. 4 shows the results for the PO administration of Compound A at 1 mg/kg 2 hours before the assay and IP administration of Valproic Acid at 30, 56 or 100 mg/kg 0.5 hours before the assay in CF-1 mice (Single dose groups: n=15 Compound A at 1 mg/kg, n=15 Valproic Acid at 100 mg/kg, n=7 Valproic Acid at 30 and 56 mg/kg; Combination dose groups: n=15 Compound A+Valproic Acid 100 mg/kg, n=8 Compound A+Valproic Acid 30 and 56 mg/kg; n=15 vehicle). Top graph: presence or absence of hindlimb tonic extensor component during the tonic seizure per animal Bottom graph: comparison of single dose or combination dosing of Compound A and Valproic Acid. Four asterisks indicates p<0.0001.

FIG. 5 shows the PK/PD of Valproic Acid with and without 1 mg/kg Compound A in the mouse MES assay. The solid curves indicate the best fit to the concentration-response curves for valproic acid with and without 1 mg/kg Compound A. The curve was fit with a maximum of 100% seizures for valproic acid alone and 73.3% with 1 mg/kg Compound A to reflect the efficacy of Compound A alone at this dose. The effect of co-dosing with Compound A was to reduce the IC₅₀ for Valproic Acid from 1440 μM to 608 μM.

FIG. 6 shows the results for the PO administration of Compound A at 1 or 1.5 mg/kg 2 hours before the assay and IP administration of Levetiracetam at 120 or 150 mg/kg 2 hours before the assay in mice. Top graph: presence or absence of hindlimb tonic extensor component during the tonic seizure per animal Bottom graph: comparison of single dose or combination dosing of Compound A and Levetiracetam.

FIG. 7 shows the results for the PO administration of Compound A at 0.25, 0.75, 1, 1.5 or 2.5 mg/kg 2 hours before the assay and IP administration of Phenytoin at 2 mg/kg 2 hours before the assay. Single dose: Compound A, n=8 per dose, Phenytoin 2 mg/kg, n=24; Combination dose: n=8 per group, n=24 vehicle. Top graph: presence or absence of hindlimb tonic extensor component during the tonic seizure per animal Bottom graph: comparison of single dose or combination dosing of Compound A and Phenytoin. Two asterisks indicates 0.001<p<0.01.

FIG. 8 shows the PK/PD of Compound A with and without 2 mg/kg Phenytoin in the mouse MES assay. The solid curves indicate the best fit to the concentration-response curves for Compound A with and without 2 mg/kg Phenytoin. The curve was fit with a maximum of 94.1% seizures for Compound A alone and 75% with 2 mg/kg Phenytoin to reflect the efficacy of Phenytoin alone at 2 mg/kg. These top values are indicated by horizontal dashed lines. The effect of Phenytoin co-dosing with Compound A is to reduce the IC₅₀ for Compound A from 147 nM to 39.7 nM.

FIG. 9 shows the fraction of mice seizing versus various doses of Compound A and that Compound A provides dose-dependent efficacy in the mouse AC-MES assay (A, B, and C). Compound A was administered PO at 1, 3, 5, 7.5, and 10 mg/kg 0.5 hours before the assay in CF-1 mice (groups: n=8 Compound A at 1 mg/kg, n=8 Compound A at 3 mg/kg, n=16 Compound A at 5 mg/kg, n=7 Compound A at 7.5 mg/kg, n=17 Compound A at 10 mg/kg; n=24 vehicle). Results are expressed as the fraction of animals seizing in any given dose group. The fraction of animals seizing in Compound A-treated groups was significantly different from the vehicle-treated group in all three studies (respective p-values are shown in FIG. 9A-C). Plasma (D) and brain tissue (E) concentration-response curves of Compound A based on the Hill Langmuir equation show that efficacy is concentration-dependent. The concentration response curve analysis of Compound A showed an EC₅₀ of 0.30 μM for plasma and an EC₅₀ of 0.47 μM for brain tissue. Terminal plasma and brain samples were obtained at 0.5 hours after dosing with Compound A. Each data point in D and E represents the fraction of animals seizing at the mean concentration level for each dose group.

FIG. 10 shows that Lacosamide provides dose-dependent efficacy in the mouse AC-MES assay (A, B, C, and D). Lacosamide was administered PO at 6, 8, 10, and 20 mg/kg 2 hours before the assay in CF-1 mice (groups: n=16 Lacosamide at 6 mg/kg; n=8 Lacosamide at 8 mg/kg; n=16 Lacosamide at 10 mg/kg; n=8 Lacosamide at 20 mg/kg; n=32 vehicle). Results are expressed as the fraction of animals seizing in any given dose group (n=8). In Study 2D, the fraction of animals seizing in the 20 mg/kg Lacosamide-treated group was significantly different from the vehicle-treated group (the p-value is shown in FIG. 10A). Plasma (E) and brain tissue (F) concentration-response curves of Lacosamide based on the Hill Langmuir equation show that efficacy is concentration-dependent. The concentration response curve analysis of Lacosamide showed an EC₅₀ for plasma of 21.6 μM and an EC₅₀ for brain tissue of 22.2 μM. Terminal plasma and brain samples were obtained at 2 hours after dosing with Lacosamide. Each data point in E and F represents the fraction of animals seizing at the mean concentration level for each dose group (n=8).

FIG. 11 shows the results from a combination of Compound A and Lacosamide in the mouse AC-MES assay (A). Compound A was administered PO at 3 mg/kg 0.5 hours before the assay; Lacosamide was administered PO at 10 mg/kg 2 hours before the assay in CF-1 mice (single dose groups: n=8 Compound A at 3 mg/kg, n=8 Lacosamide at 10 mg/kg; combination dose group: n=8 Compound A at 3 mg/kg+Lacosamide at 10 mg/kg; n=8 vehicle). Results are expressed as the fraction of animals seizing in any given dose group (n=8). The difference between the Lacosamide-treated group and the combination group was statistically significant (p-values are shown in FIG. 11A). The pharmacokinetic-pharmacodynamic relationship (PK/PD) of Compound A with and without 10 mg/kg Lacosamide and PK/PD of Lacosamide with and without 3 mg/kg Compound A in the mouse AC-MES assay is shown in B and C. Each data point in B and C represents an individual concentration obtained from a single animal and whether tonic seizures were observed in the animal Terminal plasma and brain samples were obtained at 0.5 hours after PO dosing with Compound A and 2 hours after PO dosing with Lacosamide.

FIG. 12 shows dose responses of Compound A in the mouse 6 Hz psychomotor assay 1 hour following a single oral dose. PO dosing of Compound A 1 hour prior to the seizure assay showed dose-dependent efficacy (A) with a significant effect reached at a dose of 8 mg/kg (**p=0.0081 vs. vehicle). The dose response curve (B) based on the Langmuir Hill equation projects an ED₅₀ of 6.48 mg/kg and an ED₂₀ of 4.13 mg/kg at a Hill coefficient n=−3.09.

FIG. 13 shows the concentration response of Compound A in the mouse 6 Hz psychomotor assay 1 hour following a single oral dose. Individual animal plasma (A) and brain (B) exposures at 1 hour following a single PO dose of Compound A demonstrate a clear relationship between Compound A tissue concentration and efficacy in the 6 Hz seizure assay. The animal that experienced tremor and was cold to the touch had the highest exposure and is marked with a circle. The plasma (C) and brain (D) concentration response curves based on the Hill Langmuir equation project a plasma EC₅₀ of 0.35 μM at a Hill coefficient of n=−1.95, and a brain EC₅₀ of 0.54 μM at a Hill coefficient of n=−2.17.

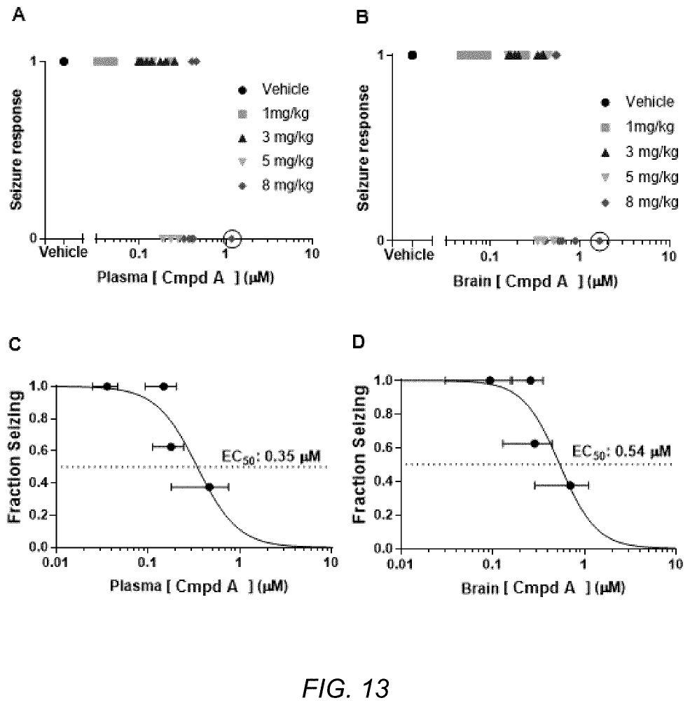

FIG. 14 shows the efficacy of Compound A and Levetiracetam alone and combined in the 6 Hz psychomotor seizure assay 1 hour post dosing. (A) Compound A (dosed orally) and Levetiracetam (dosed intraperitoneally) alone demonstrated different levels of efficacy in each of the two studies (no protection in Study 3B and up to 25% protection in Study 3C). In each study, the combination of Compound A and Levetiracetam resulted in significantly increased protection from seizure compared to either compound alone, and compared to vehicle (*p=0.034; **p<0.01; ***p=0.0002). (B) When both studies are combined, maximal efficacy with either compound alone was reached with Levetiracetam at 14/16 seizing, while the combination of both compounds resulted in 5/15 animals seizing. The combination of Compound A and Levetiracetam thus protected 66.7% of animals from seizure, which was significantly different from vehicle (****p<0.0001), and from either compound alone (***p<0.001).

FIG. 15 shows pharmacokinetics of Compound A and Levetiracetam alone and combined at 1 hour post dosing in CD-1 mice. (A) Comparison of the plasma concentrations of Compound A (dosed orally) and Levetiracetam (dosed intraperitoneally) across all experimental groups reveals that a 12-fold higher plasma concentration of Compound A was reached at 1 hour post dosing in Study 3C compared to Study 3B. Plasma concentrations of Levetiracetam were comparable between the two studies. Administration of Compound A and Levetiracetam in combination (Combo) did not significantly change exposure of either compound. One animal in the combination group in Study 3C had no measurable concentration of Levetiracetam in the plasma and was excluded (not shown). (B) Comparison of the brain concentrations of Compound A (dosed orally) and Levetiracetam (dosed intraperitoneally) across all experimental groups reveals that an 11-fold higher brain concentration of Compound A was reached at 1 hour post dosing in Study 3C compared to Study 3B. Brain concentrations of Levetiracetam were comparable between the two studies. Administration of Compound A and Levetiracetam in combination (Combo) did not significantly change exposure of either compound. One animal in the combination group in Study 3C had no measurable concentration of Levetiracetam in the brain and was excluded (not shown).

FIG. 16 shows pharmacokinetic-pharmacodynamic shifts in the efficacy of Compound A and Levetiracetam after combination dosing. LEV: Levetiracetam. When Compound A (dosed orally) is administered in combination with Levetiracetam (dosed intraperitoneally; open circles), the fraction of animals seizing is reduced compared with Compound A administered alone (filled circles), although both groups have similar concentrations of Compound A in plasma (A) and brain (B). Likewise, the fraction of animals seizing is reduced in the combination dose group (open squares) compared to the administration of Levetiracetam alone (filled squares), although both groups have similar concentrations of Levetiracetam in plasma (C) and brain (D).

FIG. 17 shows the dose and concentration response of Compound A following a single oral dose in the mouse AC-MES assay. Compound A and vehicle group data are shown from the studies indicated. Compound A shows dose-dependent efficacy in the mouse AC-MES assay (A, B, C, and D). Compound A was administered PO at 1, 2, 3, 5, 7.5, and 10 mg/kg 0.5 hours before the assay in CF-1 mice (groups: n=8 Compound A at 1 mg/kg, n=8 Compound A at 2 mg/kg, n=8 Compound A at 3 mg/kg, n=16 Compound A at 5 mg/kg, n=7 Compound A at 7.5 mg/kg, n=17 Compound A at 10 mg/kg; n=24 vehicle). Results are expressed as the fraction of animals seizing in any given dose group. The fraction of animals seizing in Compound A-treated groups was significantly different from the vehicle-treated group in 3/4 studies (respective p-values are shown). Plasma (E) and brain tissue (F) concentration-response curves of Compound A based on the Hill Langmuir equation show that efficacy was concentration-dependent. The concentration response curve analysis of Compound A showed an EC₅₀ of 0.296 μM for plasma and an EC₅₀ of 0.471 μM for brain tissue. Terminal plasma and brain samples were obtained at 0.5 hours after dosing with Compound A. Each data point in E and F represents the fraction of animals seizing at the mean concentration level for each dose group.

FIG. 18 shows the dose and concentration response of Cenobamate following a single oral dose in the mouse AC-MES assay. Cenobamate and vehicle group data are shown from the studies indicated. Cenobamate shows dose-dependent efficacy in the mouse AC-MES assay (A, B, and C). Cenobamate was administered PO at 3, 5, 7.5, 10, and 30 mg/kg 2 hours before the assay in CF-1 mice (groups: n=15 Cenobamate at 3 mg/kg; n=15 Cenobamate at 5 mg/kg; n=8 Cenobamate at 7.5 mg/kg; n=8 Cenobamate at 10 mg/kg; n=8 Cenobamate at 30 mg/kg; n=24 vehicle). Results are expressed as the fraction of animals seizing in any given dose group. The fraction of animals seizing in the cenobamate-treated group was significantly different in 2/3 studies from the vehicle-treated group (the p-value is shown). Plasma (D) and brain tissue (E) concentration-response curves of Cenobamate based on the Hill Langmuir equation show that efficacy is concentration-dependent. The concentration response curve analysis of Cenobamate showed an EC₅₀ for plasma of 70.5 μM and an EC₅₀ for brain tissue of 25.2 μM. Terminal plasma and brain samples were obtained at 2 hours after dosing with Cenobamate. Each data point in D and E represents the fraction of animals seizing at the mean concentration level for each dose group.

FIG. 19 shows the anticonvulsant effects of Compound A and Cenobamate in combination in the mouse AC-MES assay. A combination of Compound A and Cenobamate in the mouse AC-MES assay (A and B). Compound A was administered PO at 0.5, 1, and 2 mg/kg 0.5 hours before the assay; Cenobamate was administered PO at 5 mg/kg 2 hours before the assay in CF-1 mice (single dose groups: n=8 Compound A at 2 mg/kg, n=8 Cenobamate at 5 mg/kg; combination dose group: n=8 Compound A at 2 mg/kg+Cenobamate at 5 mg/kg; n=8 Compound A at 1 mg/kg+Cenobamate at 5 mg/kg; n=8 Compound A at 0.5 mg/kg+Cenobamate at 5 mg/kg; n=8 vehicle). Results are expressed as the fraction of animals seizing in any given dose group. In study 4F, the difference between the Cenobamate-treated group and the combination group was statistically significant, whereas the difference between the Compound A-treated group and combination group was not statistically significant. In study 4G, the difference between the vehicle group and the combination groups was statistically significant. P-values are shown. The pharmacokinetic-pharmacodynamic relationship (PK/PD) of Compound A with and without Cenobamate by plasma and brain concentration is shown in C and D. Each data point in C and D represents the fraction of animals seizing at the mean concentration level for each dose group. Terminal plasma and brain samples were obtained at 0.5 hours after PO dosing with Compound A and 2 hours after PO dosing with Cenobamate.

### 4. DETAILED DESCRIPTION

The present disclosure relates to, among other things, novel and improved methods and uses for treating a seizure disorder in a human in need thereof, comprising conjointly administering Compound A and an antiseizure medication (ASM) to the human, including by oral administration.

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### 4.1. Definitions

As used in the specification and appended claims, unless specified to the contrary, the following terms and abbreviations have the meaning indicated:

“Compound A” refers to the compound having the following formula:

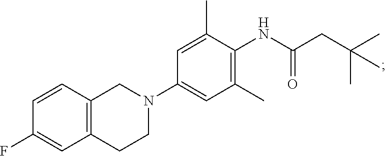 [SMILES: Cc1cc(N2CCc3cc(F)ccc3C2)cc(C)c1NC(=O)CC(C)(C)C]

and having a chemical name of N-[4-(6-fluoro-3,4-dihydro-1H-isoquinolin-2-yl)-2,6-dimethylphenyl]-3,3-dimethylbutanamide. Preparation of Compound A and its use as a Kv7.2/Kv7.3 (KCNQ2/3) opener is disclosed in U.S. Pat. Nos. 8,293,911 and 8,993,593 as well as U.S. application Ser. Nos. 16/409,684 and 16/410,851. Compound A is different from most known antiseizure medications in that it potentiates and enhances opening of the voltage-gated potassium channels Kv7.2 and Kv7.3 (Kv7.2/Kv7.3), which are important in controlling neuronal excitability. Compound A is used in the methods and uses described herein. It will be understood that any reference to Compound A or any of the ASMs mentioned in the disclosure also includes pharmaceutically acceptable salts thereof (e.g., valproic acid may also be sodium valproate or valproate semi-sodium forms).

“Conjointly administering” refers herein to any form of administration of two or more different therapeutic compounds such that the second administered compound is administered while the first administered therapeutic compound is still effective in the body (e.g., the two compounds are simultaneously effective in the patient, which may include additive or synergistic effects of the two compounds). For example, Compound A and the antiseizure medications disclosed herein can be administered either in the same formulation or in a separate formulation, either concomitantly or sequentially. In certain embodiments, Compound A and the antiseizure medications disclosed herein can be administered within one hour, 12 hours, 24 hours, 36 hours, 48 hours, 72 hours, or a week of one another. Thus, an individual who receives such treatment can benefit from a combined effect of the different therapeutic compounds.

“Decreased neuronal excitation” or “decreased neuronal excitability” as used herein refers to a level of neuronal cell activity that is lessened to some degree toward a normal physiological state that would be observed in the absence of the seizure disorder in a patient. Particular agents which decrease neuronal excitability include agents which act upon channels and receptors expressed on neuronal cells to directly decrease the level of excitability of the neuron. Conversely, the agent may act indirectly to decrease the level of excitability of the neuron by initiating a cascade of cellular events, the downstream effects of which result in decreased neuronal excitability. Certain antiseizure medications that decrease neuronal excitability to restore physiological levels of neuronal activity are described herein.

“Increased neuronal inhibition” as used herein refers to a level of neuronal inhibition that is increased to some degree toward a normal physiological state that would be observed in the absence of the seizure disorder in a patient. Certain antiseizure medications, such as GABAergic agents, that increase neural inhibition to restore physiological levels of neuronal activity are described herein.

“Seizure disorder(s)” refers to seizures and disorders associated with seizures such as partial onset (focal) seizures, photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures+, Rett syndrome, multiple sclerosis, Alzheimer's disease, autism, ataxia, hypotonia and paroxysmal dyskinesia. In certain embodiments, the term “seizure disorder” refers to focal onset epilepsy, also known as partial onset (focal) epilepsy.

“Therapeutically effective amount” as used herein refers to an amount of Compound A, an amount of ASM, or both an amount of Compound A and an amount of an ASM that is sufficient to treat the stated disease, disorder, or condition or have the desired stated effect on the disease, disorder, or condition or one or more mechanisms underlying the disease, disorder, or condition in a human subject. In certain embodiments, when Compound A is administered conjointly with an ASM for the treatment of a seizure disorder, therapeutically effective amount refers to both an amount of Compound A and an amount of the ASM which, upon conjoint administration to a human, treats or ameliorates a seizure disorder in the human, or exhibits a detectable therapeutic effect in the human having a seizure disorder. The effect can be detected by, for example, a reduction in the number of seizure episodes or by the reduction of the severity of seizure episodes.

“Treatment” as used herein refers to therapeutic applications associated with conjointly administering Compound A and an ASM that ameliorate the indicated disease, disorder, or condition or one or more underlying mechanisms of said disease, disorder, or condition, including slowing or stopping progression of the disease, disorder or condition or one or more of the underlying mechanisms in a human subject. In certain embodiments, when Compound A and an ASM are conjointly administered for the treatment of a seizure disorder, treatment refers to therapeutic applications to slow or stop progression of a seizure disorder and/or reversal of a seizure disorder. Reversal of a seizure disorder differs from a therapeutic application which slows or stops a seizure disorder in that with a method of reversing, not only is progression of a seizure disorder stopped, cellular behavior is moved to some degree toward a normal state that would be observed in the absence of the seizure disorder. In some embodiments, the treatment of a seizure disorder comprising the conjoint administration of Compound A with an ASM is accompanied by an alteration of the cellular activity of one or more Kv7 potassium channels (e.g., Kv7.2, Kv7.3, Kv7.4, and/or Kv7.5, particularly Kv7.2 and/or Kv7.3, optionally over Kv7.1) toward a normal level that would be observed in the absence of the seizure disorder.

“Under fed conditions” refers to the condition of having consumed food during the time period between from about 4 hours prior to the oral administration of an effective amount (e.g., within the therapeutically effective dose range) of Compound A to about 4 hours after the administration of Compound A. The food may be a solid, liquid, or mixture of solid and liquid food with sufficient bulk and fat content that it is not rapidly dissolved and absorbed in the stomach. In some instances, the food is a meal, such as breakfast, lunch, dinner or, alternatively, baby food (e.g., formula or breast milk). The therapeutically effective amount of Compound A may be orally administered to the subject, for example, between about 30 minutes prior to about 2 hours after eating a meal, most advantageously, the dosage unit of Compound A is orally administered during a meal or within 15 minutes after eating a meal.

“Under fasted conditions” refers to the condition of not having consumed food during the time period between from at least 4 hours prior to the oral administration of a therapeutically effective amount of Compound A to about 4 hours after administration of Compound A.

### 4.2. Embodiments

In some embodiments, the present disclosure is directed to a method of treating a seizure disorder in a human in need thereof, comprising conjointly administering (e.g., orally) Compound A and an antiseizure medication (ASM) to the human in amounts that are therapeutically effective when conjointly administered. Likewise, in some embodiments, the present disclosure is directed to the use of Compound A and an ASM, in amounts that are therapeutically effective when conjointly administered, in treating a seizure disorder in a human in need thereof. Conjoint administration contemplates that Compound A can be administered simultaneously, prior to, or after administration of the ASM. In certain instances, the seizure disorder treated comprising the conjoint administration of Compound A and an ASM is focal onset epilepsy.

In further embodiments when a seizure disorder is treated herein, the seizure disorder is selected from focal onset epilepsy, photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures+, Rett syndrome, multiple sclerosis, Alzheimer's disease, autism, ataxia, hypotonia and paroxysmal dyskinesia. In certain embodiments, the seizure disorder is focal onset epilepsy, also known as partial onset (focal) epilepsy.

In some embodiments, the present disclosure is directed to a method of reducing the amount of an ASM that is required for therapeutic efficacy in a human suffering from a seizure disorder, comprising administering (e.g., orally) to the human, conjointly with the ASM, an amount of Compound A that is effective to achieve such reduction when administered with the ASM. Likewise, in some embodiments, the present disclosure is directed to the use of Compound A in reducing the amount of an ASM that is required for therapeutic efficacy in a human suffering from a seizure disorder, such as by administering to the human, conjointly with the ASM, an amount of Compound A that is effective to achieve such reduction when administered with the ASM. For instance, in some embodiments, the present disclosure provides a method of treating a seizure disorder in a subject (e.g., a human) in need thereof, comprising conjointly administering (e.g., orally) Compound A and an ASM, wherein the amount of the ASM administered is less than the amount of the ASM that would be needed to achieve the same or a similar reduction in the number of seizure episodes or reduction of the severity of seizure episodes in the absence of administering Compound A. In some embodiments, the ASM is valproic acid, levetiracetam, phenytoin, lacosamide, cenobamate or a combination thereof, particularly valproic acid.

In some embodiments, the present disclosure is directed to a method of reducing the amount of Compound A that is required for therapeutic efficacy in a human suffering from a seizure disorder, comprising administering (e.g., orally) to the human, conjointly with Compound A, an amount of an ASM that is effective to achieve such reduction when administered with Compound A. Likewise, in some embodiments, the present disclosure is directed to the use of an ASM in reducing the amount of Compound A that is required for therapeutic efficacy in a human suffering from a seizure disorder, such as by administering to the human, conjointly with Compound A, an amount of the ASM that is effective to achieve such reduction when administered with Compound A. For instance, in some embodiments, the present disclosure provides a method of treating a seizure disorder in a subject (e.g., a human) in need thereof, comprising conjointly administering (e.g., orally) Compound A and an ASM, wherein the amount of Compound A administered is less than the amount of Compound A that would be needed to achieve the same or a similar reduction in the number of seizure episodes or reduction of the severity of seizure episodes in the absence of administering the ASM. In some embodiments, the ASM is valproic acid, levetiracetam, phenytoin, lacosamide, cenobamate or a combination thereof, particularly phenytoin.

In some embodiments, the methods and uses described herein of treating a seizure in a human in need thereof, of reducing the amount of an ASM required for therapeutic efficacy or of reducing the amount of Compound A required for therapeutic efficacy, comprise enhancing the opening of a Kv7 potassium channel in the human.

In certain embodiments, the present disclosure provides a method or use comprising opening or enhancing the opening of a Kv7 potassium channel, such as the Kv7.2, Kv7.3, Kv7.4, and/or Kv7.5 potassium channel, particularly the Kv7.2/Kv7.3 (KCNQ2/3) potassium channel in a human in need thereof by administering an effective amount of Compound A conjointly with an ASM. In some such embodiments, the human has a seizure disorder, such as those described herein.

In certain instances, the method or use described herein comprises selectively opening or enhancing the opening of a Kv7 potassium channel, such as one or more of Kv7.2, Kv7.3, Kv7.4, or Kv7.5 over Kv7.1. In some embodiments, the method or use is selective for Kv7.2, over Kv7.1. In other embodiments, the method or use is selective for Kv7.3, over Kv7.1. In yet other embodiments, the method or use is selective for Kv7.4, over Kv7.1. In yet further other embodiments, the method or use is selective for Kv7.5, over Kv7.1. In certain embodiments, the method or use is selective for Kv7.2 and Kv7.3, over Kv7.1. In certain embodiments, the method or use is selective for Kv7.2 and Kv7.3 over other Kv7 potassium channels. In certain embodiments, the method or use is selective for Kv7.2 and Kv7.3 over Kv7.4 and Kv7.5.

In one embodiment, the methods and uses described herein, such as the method of or use in treating a seizure disorder in a human in need thereof, is achieved by administering (e.g., orally) an amount of Compound A conjointly with an ASM, such as from about 0.01 mg/kg to about 2.0 mg/kg of Compound A. More specific representative amounts of Compound A include 0.05 mg/kg, 0.10 mg/kg, 0.20 mg/kg, 0.30 mg/kg, 0.40 mg/kg, 0.5 mg/kg, 0.6 mg/kg, 0.7 mg/kg, 0.80 mg/kg, 0.90 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg and 2.0 mg/kg, or any range of amounts created by using two of the aforementioned amounts as endpoints. In some aspects, the method or use includes administering (e.g., orally) 0.03-1.0 mg/kg of Compound A conjointly with a disclosed amount of an ASM. In some aspects, the method includes administering (e.g., orally) 0.05-0.5 mg/kg of Compound A conjointly with a disclosed amount of an ASM.

In some embodiments, the methods and uses described herein, such as the method of or use in treating a seizure disorder in a human in need thereof, is achieved by administering (e.g., orally) an amount of Compound A conjointly with an ASM, such as 2 to 200 mg of Compound A in a single or divided doses. For example, the method can include administering Compound A (e.g., orally), in a single or divided doses, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, or about 200 mg or administering (e.g., orally) any range of amounts created by using two of the aforementioned amounts as endpoints. In some aspects, the method or use includes oral administration of 2 to 100 or 5 to 50 mg of Compound A in a single or divided doses conjointly with a disclosed amount of an ASM. In some aspects, method or use includes the oral administration of a single or divided dose of 5, 10, 15, 20, or 25 mg of Compound A conjointly with a disclosed amount of an ASM. In some aspects, the method or use includes oral administration of a single or divided dose of 20 mg of Compound A conjointly with a disclosed amount of an ASM.

In some aspects, the methods and uses described herein, such as the method of or use in treating a seizure disorder in a human in need thereof, is achieved by administering (e.g., orally) at least 10 mg of Compound A, such as at least 20, 30, 40, 50, 75, or 100 mg of Compound A conjointly with a disclosed amount of an ASM. In some embodiments, the methods and uses described herein, such as the method of or use in treating a seizure disorder in a human in need thereof, is achieved by administering (e.g., orally) at least 50 mg of Compound A, such as at least 75, 100, 125, 150, 175, or 200 mg of Compound A conjointly with a disclosed amount of an ASM.

In some embodiments, the methods and uses described herein, such as the method of or use in treating a seizure disorder in a human in need thereof, is achieved by administering (e.g., orally) an amount of Compound A per day, such as 5 to 1000 mg of Compound A per day, such as 5 to 500 mg or 5 to 250 mg of Compound A per day conjointly with an ASM. For example, the method or use can include administering (e.g., orally) about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, or about 1000 mg of Compound A per day, or administering (e.g., orally) per day a range of amounts created by using two of the aforementioned amounts as endpoints. In some aspects, the method or use includes orally administering 5 to 250 mg of Compound A per day, such as 10, 15, 20, 25, 30, 35, or 40 mg to 75, 100, 125, 150, 175, or 200 mg of Compound A per day, including 20 to 150 mg per day conjointly with a disclosed amount of an ASM. In some aspects, the oral administration includes 50, 75, 100, or 125 mg of Compound A per day, such as 100 mg per day conjointly with a disclosed amount of an ASM.

In certain instances, the above daily doses of Compound A are administered (e.g., orally) as multiple doses per day, such as in two, three, four, or five doses per day. For example, a daily dose of 100 mg, may be administered in five 20 mg, four 25 mg, three 33.3 mg, or two 50 mg doses throughout the day conjointly with a disclosed amount of an ASM.

In some embodiments, the above daily doses of Compound A are administered (e.g., orally) as a single dose of Compound A or as a single dose of Compound A conjointly with a single dose of an ASM. For example, about 5, 10, 15, 20, 25, or 30 mg to about 50, 65, 75, 100, 125, or 150 mg of Compound A per day can be orally administered as a single dose, including 10-25 mg, 10-30 mg, and 10-40 mg per day as a single dose, such as 10-25 mg per day as a single dose. Relatedly, any of the doses of Compound A discussed in the preceding paragraphs may be included in a unit dosage form.

In some embodiments, the above doses of Compound A are administered (e.g., orally) as multiple doses per week, such as in two, three, four, five, ten, fifteen, or twenty doses per week. For example, a weekly dose of 100 mg, may be administered in five 20 mg, four 25 mg, or two 50 mg doses throughout the week conjointly with a disclosed amount of an ASM.

In some embodiments, the methods and uses described herein, when using the daily dosing disclosed herein, achieve a steady state for Compound A within 6 to 9 days, such as in about 1 week.

In some embodiments, the methods and uses described herein, such as the method of or use in treating a seizure disorder in a human in need thereof, is achieved by administering (e.g., orally) an amount of an ASM conjointly with Compound A, such as from about 0.01 mg/kg to about 2.0 mg/kg of ASM. More specific representative amounts of ASM include 0.05 mg/kg, 0.10 mg/kg, 0.20 mg/kg, 0.30 mg/kg, 0.40 mg/kg, 0.5 mg/kg, 0.6 mg/kg, 0.7 mg/kg, 0.80 mg/kg, 0.90 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg and 2.0 mg/kg, or any range of amounts created by using two of the aforementioned amounts as endpoints. In some aspects, the method or use includes administering (e.g., orally) 0.03-1.0 mg/kg of an ASM conjointly with Compound A. In some aspects, the method includes administering (e.g., orally) 0.05-0.5 mg/kg of an ASM conjointly with Compound A.

In some embodiments, the methods and uses described herein, such as the method of or use in treating a seizure disorder in a human in need thereof, is achieved by administering (e.g., orally) an amount of an ASM conjointly with Compound A, such as 2 to 200 mg of an ASM in a single or divided doses. For example, the method can include administering an ASM (e.g., orally), in a single or divided doses, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, or about 200 mg or administering (e.g., orally) any range of amounts created by using two of the aforementioned amounts as endpoints. In some aspects, the method or use includes oral administration of 2 to 100 or 5 to 50 mg of an ASM in a single or divided doses conjointly with Compound A. In some aspects, method or use includes the oral administration of a single or divided dose of 5, 10, 15, 20, or 25 mg of an ASM conjointly with Compound A. In some aspects, the method or use includes oral administration of a single or divided dose of 20 mg of an ASM conjointly with a disclosed amount of Compound A.

In some aspects, the methods and uses described herein, such as the method of or use in treating a seizure disorder in a human in need thereof, is achieved by administering (e.g., orally) at least 10 mg of an ASM, such as at least 20, 30, 40, 50, 75, or 100 mg of an ASM conjointly with Compound A. In some embodiments, the methods and uses described herein, such as the method of or use in treating a seizure disorder in a human in need thereof, is achieved by administering (e.g., orally) at least 50 mg of an ASM, such as at least 75, 100, 125, 150, 175, or 200 mg of an ASM conjointly with Compound A.

In some embodiments, the methods and uses described herein, such as the method of or use in treating a seizure disorder in a human in need thereof, is achieved by administering (e.g., orally) an amount of an ASM per day, such as 5 to 1000 mg of an ASM per day, such as 5 to 500 mg or 5 to 250 mg of an ASM per day conjointly with Compound A. For example, the method or use can include administering (e.g., orally) about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, or about 1000 mg of an ASM per day, or administering (e.g., orally) per day a range of amounts created by using two of the aforementioned amounts as endpoints. In some aspects, the method or use includes orally administering 5 to 250 mg of an ASM per day, such as 10, 15, 20, 25, 30, 35, or 40 mg to 75, 100, 125, 150, 175, or 200 mg of an ASM per day, including 20 to 150 mg per day conjointly with Compound A. In some aspects, the oral administration includes 50, 75, 100, or 125 mg of an ASM per day, such as 100 mg per day conjointly with Compound A.

In certain instances, the above daily doses of an ASM are administered (e.g., orally) as multiple doses per day, such as in two, three, four, or five doses per day. For example, a daily dose of 100 mg, may be administered in five 20 mg, four 25 mg, three 33.3 mg, or two 50 mg doses throughout the day conjointly with Compound A.

In some embodiments, the above daily doses of an ASM are administered (e.g., orally) as a single dose conjointly with Compound A. For example, about 5, 10, 15, 20, 25, or 30 mg to about 50, 65, 75, 100, 125, or 150 mg of an ASM per day can be orally administered as a single dose, including 10-25 mg, 10-30 mg, and 10-40 mg per day as a single dose, such as 10-25 mg per day as a single dose conjointly with Compound A. Relatedly, any of the doses of the ASM discussed in the preceding paragraphs may be included in a unit dosage form.

In some embodiments of the present methods and uses described herein, the ASM is conjointly administered with Compound A is one or more benzodiazepines (e.g., chlorazepate, clobazam, clonazepam, diazepam, lorazepam, nitrazepam, etc.), carbamazepine, cenobamate, felbamate, gabapentin, lacosamide, lamotrigine, levetiracetam, oxcarbazepine, phenobarbital, phenytoin, pregabalin, rufinamide, tiagabine, topiramate, valproic acid, vigabatrin, or zonisamide. In some embodiments, the ASM conjointly administered with Compound A is valproic acid, levetiracetam, phenytoin, lacosamide, cenobamate or a combination thereof.

In some embodiments of the present methods and uses described herein, the ASM treats seizures disorder in a patent by not enhancing the opening of a Kv7 potassium channel in the human (e.g., treats a seizure disorder in a patent by a different mechanism than Compound A). In some embodiments, the ASM decreases neuronal excitation by blocking a sodium channel in the human. ASMs that are known to be sodium channel blockers include, for example, carbamazepine, lacosamide, lamotrigine, oxcarbazepine, phenytoin, rufinamide, topiramate, and zonisamide. In some embodiments, the sodium channel blocker inhibits neuronal action potential firing and transmission by promoting inactivation and reducing contributions to electrical activity at the axon initial segment (AIS) as well as on the axon itself.

In some embodiments, the present disclosure is directed to a method of treating a seizure disorder in a human in need thereof, comprising conjointly administering (e.g., orally) Compound A and carbamazepine to the human in amounts that are therapeutically effective when conjointly administered.

In some embodiments, the present disclosure is directed to a method of treating a seizure disorder in a human in need thereof, comprising conjointly administering (e.g., orally) Compound A and lacosamide to the human in amounts that are therapeutically effective when conjointly administered.

In some embodiments, the present disclosure is directed to a method of treating a seizure disorder in a human in need thereof, comprising conjointly administering (e.g., orally) Compound A and lamotrigine to the human in amounts that are therapeutically effective when conjointly administered.

In some embodiments, the present disclosure is directed to a method of treating a seizure disorder in a human in need thereof, comprising conjointly administering (e.g., orally) Compound A and oxcarbazepine to the human in amounts that are therapeutically effective when conjointly administered.

In some embodiments, the present disclosure is directed to a method of treating a seizure disorder in a human in need thereof, comprising conjointly administering (e.g., orally) A and phenytoin to the human in amounts that are therapeutically effective when conjointly administered.

In some embodiments, the present disclosure is directed to a method of treating a seizure disorder in a human in need thereof, comprising conjointly administering (e.g., orally) Compound A and rufinamide to the human in amounts that are therapeutically effective when conjointly administered.

In some embodiments, the present disclosure is directed to a method of treating a seizure disorder in a human in need thereof, comprising conjointly administering (e.g., orally) of Compound A and topiramate to the human in amounts that are therapeutically effective when conjointly administered.

In some embodiments, the present disclosure is directed to a method of treating a seizure disorder in a human in need thereof, comprising conjointly administering (e.g., orally) of Compound A and zonisamide to the human in amounts that are therapeutically effective when conjointly administered.

In some embodiments of the present methods and uses described herein, the ASM decreases neuronal excitation by blocking a calcium channel in the human. ASMs that are known to be calcium channel blockers include, for example, gabapentin, phenobarbital, pregabalin, and zonisamide. In some embodiments, the calcium channel blocker reduces excitatory transmission by reducing presynaptic neurotransmitter release, a calcium-dependent process.

In some embodiments, the present disclosure is directed to a method of treating a seizure disorder in a human in need thereof, comprising conjointly administering (e.g., orally) Compound A and gabapentin to the human in amounts that are therapeutically effective when conjointly administered.

In some embodiments, the present disclosure is directed to a method of treating a seizure disorder in a human in need thereof, comprising conjointly administering (e.g., orally) Compound A and phenobarbital to the human in amounts that are therapeutically effective when conjointly administered.

In some embodiments, the present disclosure is directed to a method of treating a seizure disorder in a human in need thereof, comprising conjointly administering (e.g., orally) Compound A and pregabalin to the human in amounts that are therapeutically effective when conjointly administered.

In some embodiments, the present disclosure is directed to a method of treating a seizure disorder in a human in need thereof, comprising conjointly administering (e.g., orally) Compound A and gabapentin to the human in amounts that are therapeutically effective when conjointly administered.

In some embodiments of the present methods and uses described herein, the ASM decreases neuronal excitation by binding to synaptic vesicle glycoprotein 2A (SV2A) in the human. ASMs that are known to bind to SV2A include, for example, levetiracetam. In some embodiments, the SV2A binder reduces excitatory transmission by reducing presynaptic neurotransmitter release.

In some embodiments, the present disclosure is directed to a method of treating a seizure disorder in a human in need thereof, comprising conjointly administering (e.g., orally) Compound A and levetiracetam to the human in amounts that are therapeutically effective when conjointly administered.

In some embodiments of the present methods and uses described herein, the ASM increases neuronal inhibition in the human. ASMs that are known to increase neuronal inhibition include, for example, benzodiazepines (e.g., chlorazepate, clobazam, clonazepam, diazepam, lorazepam, nitrazepam, etc.), felbamate, phenobarbital, tiagabine, topiramate, valproic acid, and vigabatrin. In some embodiments, the ASM is a glutamatergic agent. In some aspects, the glutamatergic agent reduces the effects of this neurotransmitter on AMPA or NMDA receptors on the postsynaptic membrane. In some embodiments, the glutamatergic agent is carbamazepine, felbamate, lamotrigine, pregabalin, phenytoin, pregabalin, tiagabine, or topiramate. In other embodiments, the ASM is a GABAergic agent. In some aspects, the GABAergic agent is a benzodiazepine (e.g., chlorazepate, clobazam, clonazepam, diazepam, lorazepam, nitrazepam, etc.), felbamate, phenobarbital, tiagabine, topiramate, valproic acid, or vigabatrin. In some instances, the GABAergic agent may affect GABA receptors by direct positive allosteric modulation of GABA receptor activity (e.g., benzodiazepines). In other instances, the GABAergic agent may affect GABA receptors by indirectly increasing levels of GABA via inhibition of GABA transaminase (e.g., vigabatrin) or GABA transporter-1 (GAT1, e.g., tiagabine).

In some embodiments, the present disclosure is directed to a method of treating a seizure disorder in a human in need thereof, comprising conjointly administering (e.g., orally) Compound A and a benzodiazepine (e.g., chlorazepate, clobazam, clonazepam, diazepam, lorazepam, nitrazepam, etc.) to the human in amounts that are therapeutically effective when conjointly administered.

In some embodiments, the present disclosure is directed to a method of treating a seizure disorder in a human in need thereof, comprising conjointly administering (e.g., orally Compound A and felbamate to the human in amounts that are therapeutically effective when conjointly administered.

In some embodiments, the present disclosure is directed to a method of treating a seizure disorder in a human in need thereof, comprising conjointly administering (e.g., orally) Compound A and phenobarbital to the human in amounts that are therapeutically effective when conjointly administered.

In some embodiments, the present disclosure is directed to a method of treating a seizure disorder in a human in need thereof, comprising conjointly administering (e.g., orally) Compound A and tiagabine to the human in amounts that are therapeutically effective when conjointly administered.

In some embodiments, the present disclosure is directed to a method of treating a seizure disorder in a human in need thereof, comprising conjointly administering (e.g., orally) Compound A and topiramate to the human in amounts that are therapeutically effective when conjointly administered.

In some embodiments, the present disclosure is directed to a method of treating a seizure disorder in a human in need thereof, comprising conjointly administering (e.g., orally) Compound A and valproic acid to the human in amounts that are therapeutically effective when conjointly administered.

In some embodiments, the present disclosure is directed to a method of treating a seizure disorder in a human in need thereof, comprising conjointly administering (e.g., orally) Compound A and vigabatrin to the human in amounts that are therapeutically effective when conjointly administered.

In some embodiments of the present methods and uses, the ASM conjointly administered with Compound A is valproic acid. In some aspects, the valproic acid is administered conjointly with Compound A at any of the doses of the ASM discussed in the paragraphs above. In some embodiments, valproic acid is administered conjointly with Compound A at dose of 2-16 mg/kg to the human, for example, valproic acid may be administered at a dose of 4-12 mg/kg to the human.

In some embodiments of the present methods and uses, the ASM conjointly administered with Compound A is phenytoin. In some aspects, the phenytoin is administered conjointly with Compound A at any of the doses of the ASM discussed in the paragraphs above. In some embodiments, phenytoin is administered conjointly with Compound A at a dose of 0.05-5 mg/kg to the human, for example, phenytoin may be administered (e.g., orally) at a dose of 0.1-1 mg/kg to the human.

In some embodiments of the present methods and uses, the ASM conjointly administered with Compound A is lacosamide. In some aspects, the lacosamide is administered conjointly with Compound A at any of the doses of the ASM discussed in the paragraphs above. In some embodiments, lacosamide is administered conjointly with Compound A at a dose of 0.1-5 mg/kg to the human, for example, the lacosamide is administered (e.g., orally) at a dose of 0.5-1 mg/kg to the human.

In another embodiments of the present methods and uses, the ASM conjointly administered with Compound A is cenobamate. In some aspects, the cenobamate is administered conjointly with Compound A at any of the doses of the ASM discussed in the paragraphs above. In some embodiments, cenobamate is administered conjointly with Compound A at a dose of 0.05-5 mg/kg to the human, for example, the cenobamate is administered (e.g., orally) at a dose of 0.1-1 mg/kg to the human.

In certain embodiments of the present methods and uses, the conjoint administration of Compound A and the ASM (e.g., valproic acid, phenytoin, levetiracetam, lacosamide, or cenobamate) provides improved efficacy (e.g., increases the reduction in the number of seizure episodes or reduction of the severity of seizure episodes in the human) relative to individual administration of Compound A or the ASM alone. In certain such embodiments, the conjoint administration provides an additive effect, wherein an additive effect refers to the sum of the individual effects of administering Compound A and administering one or more ASMs. In some embodiments, the conjoint administration provides a synergistic effect, wherein a synergistic effect refers to an effect that is greater than the sum of the individual effects of administering Compound A and administering the one or more ASMs.

In additional embodiments, the above-discussed methods and uses of treating a seizure disorder by administering (e.g., orally) Compound A comprise administration of Compound A to the human under fed conditions. In some embodiments, the oral administration of Compound A to a human under fed conditions (i.e., with food or in temporal proximity to the ingestion of food) significantly enhances the bioavailability and exposure of Compound A as compared to the oral administration of Compound A to the human under fasted conditions (i.e., without food or not in temporal proximity to the ingestion of food). In some embodiments, the oral administration of Compound A to a human under fed conditions increases one or more pharmacokinetic parameters for Compound A (e.g., Cₘₐₓ, AUC_{inf}, Tₘₐₓ, t½_{λz}, etc.) as compared to when the same amount of Compound A is orally administered to the human under fasted conditions.

In certain embodiments, the methods and uses described herein administer Compound A, an ASM, or both Compound A and an ASM in the form of a pharmaceutically acceptable oral composition that comprises Compound A, an ASM, or both Compound A and an ASM, as the case may be, and one or more pharmaceutically acceptable carriers or excipients. The amount(s) of Compound A, an ASM, or both Compound A and an ASM included in these compositions may correspond to one or more of the amounts described herein. In some embodiments, the compositions are a unit dose.

Examples of pharmaceutically acceptable oral compositions that comprise Compound A, an ASM, or both Compound A and an ASM include solid formulations (such as tablets, capsules, lozenges, dragées, granules, powders, sprinkles, wafers, multi-particulates, and films), liquid formulations (such as aqueous solutions, elixirs, tinctures, tonics, slurries, suspensions, and dispersions), and aerosolized formulations (such as mists and sprays). In one embodiment, a pharmaceutically acceptable oral composition of Compound A includes a pediatric suspension or granulate. All above-noted amounts of Compound A, an ASM, or both Compound A and an ASM may be included in such formulations, e.g., a capsule comprising 5, 10, 15, 10, 25, 30, or 35 mg of Compound A, a capsule comprising 5, 10, 15, 10, 25, 30, 35, 40, 45, 50, 55, 60, 65, 75, 85, 90, 95, or 100 mg of an ASM, or a capsule comprising 5, 10, 15, 10, 25, 30, or 35 mg of Compound A and 5, 10, 15, 10, 25, 30, 35, 40, 45, 50, 55, 60, 65, 75, 85, 90, 95, or 100 mg of an ASM.

Other administration routes suitable for administration of Compound A, an ASM, or both Compound A and an ASM according to the methods and uses described herein include sublingual and buccal (e.g., with a film or other composition that dissolves in the mouth under the tongue or on the inside of the cheek), ocular (e.g., eye drops), otic (e.g., by ear drops), oral or nasal inhalation (e.g., by insufflation or nebulization), cutaneous or topical (e.g., by creams or lotions), or transdermal (e.g., by skin patches). Besides oral administration, other enteral administration routes can be used for Compound A, an ASM, or both Compound A and an ASM, including vaginal and rectal (e.g., by ointment, suppository, enema).

Examples of compositions suitable for parenteral administration of Compound A, an ASM, or both Compound A and an ASM, include sterile injectable solutions, suspensions, or dispersions, including aqueous or oleaginous preparations, particularly aqueous. In some embodiments, Compound A, an ASM, or both Compound A and an ASM is administered according to a method or use described herein in an injectable sterile aqueous formulation that includes a parenterally-acceptable diluent or solvent, such as water, Ringer's solution, isotonic sodium chloride solution, buffered aqueous solutions, and aqueous solutions containing a miscible alcohol, such as 1,3-butanediol. Additional suitable excipients for parenteral formulations of Compound A, an ASM, or both Compound A and an ASM include, mono- or di-glycerides; fatty acids, such as oleic acid and its glyceride derivatives; natural pharmaceutically-acceptable oils, such as olive oil or castor oil, including their polyoxyethylated versions; long-chain alcohol diluents or dispersants, such as alkyl celluloses, including carboxymethyl cellulose; and surfactants, such as Tweens, Spans and other emulsifying agents or bioavailability enhancers.

In another embodiment, kits are provided for oral administration of Compound A and an ASM conjointly for the treatment of a depressive disorder upon oral administration. Such kits comprise a plurality of oral dosage unit forms of Compound A, an ASM, or both Compound A and an ASM in addition to instructions for orally administering of Compound A and an ASM conjointly.

Additional embodiments and examples of the present disclosure are described herein. These embodiments and examples are illustrative and should not be construed as limiting the scope of the claimed invention.

### 5. EXAMPLES

Studies were conducted to determine the effect of Compound A with other ASMs.

### 5.1. Example 1. Anticonvulsant Effects of Compound A Alone and in Combination with Common ASMs

The interaction of Compound A with other ASMs was evaluated after oral dosing in the mouse maximal electroshock (MES) assay to determine whether some combinations were favorable or unfavorable. Efficacy was quantified by calculating the fraction of animals with a tonic hindlimb seizure following corneal stimulation.

5.1.1 Compound A

When dosed alone, Compound A (1, 2, 4, or 8 mg/kg) reduced the occurrence of tonic seizures in the MES assay at both 0.5 and 2 hr after oral dosing. The concentration response relationship was fit to a binding isotherm with an IC₅₀ of plasma concentration of 154 nM and an IC₅₀ for brain concentration of 275 nM (n=7 per dose, per time point).

Compound Preparation: Compound A was initially solubilized in DMSO. This solution was then added into a 0.5% methyl cellulose solution to create a more even and less aggregated compound suspension. Serial dilutions were prepared from the tube with the highest concentration of Compound A and the compound suspensions were further vortexed prior to animal dosing. The final concentration of DMSO was 5%, an amount with no apparent toxicity or neuroprotection in the MES assay. Phenytoin and Valproic acid were solubilized in 100% Saline (0.9%). Levetiracetam was solubilized in 0.5% methyl cellulose and 0.2% Tween 80 in deionized water.

Animals: Adult male CF-1 albino mice (25-35 g) purchased from Harlan-Envigo were used. The mice were housed four per cage and had access to filtered water and chow ad libidum, throughout the experiment.

MES Assay: Compounds were administered orally by gavage before testing unless indicated otherwise. During MES testing, a 60-Hz alternating current (50 mA) was delivered for 0.2 seconds through corneal electrodes to the mice. A drop of 0.5% Alcaine solution was placed on the eye prior to current delivery. The electrodes were subsequently placed gently onto the eyes of the animal and the electrical shock was initiated by triggering through a foot-pedal activator. The animals were restrained by hand and gently released as the shock was delivered and the seizure commenced. Animals were monitored for hind limb tonic extension as the end point for this test.

Statistical analysis: All statistics were calculated using Prism version 7 software (Graphpad Software). The method used for each experiment is indicated in the results section. Concentration-response curves were calculated as best fits to the equation

Y=Bottom+(Top−Bottom)/(1+10\hat{ }((Log IC50−X)*HillSlope))

where X was the log of plasma concentration. Unless indicated otherwise, bottom was constrained to be 0. Top was constrained to be the value determined experimentally by vehicle control measurements.

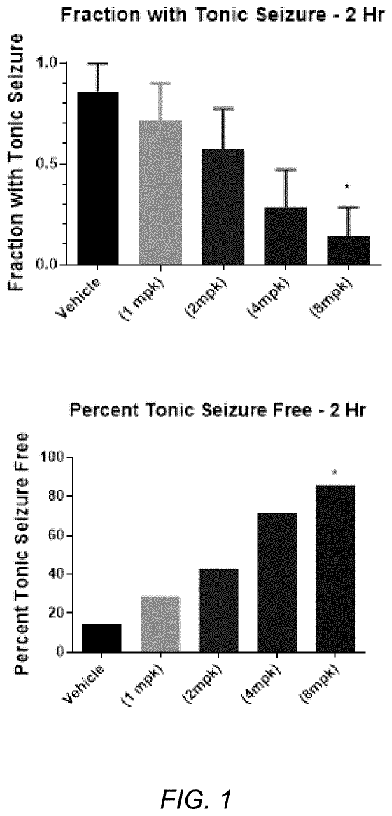

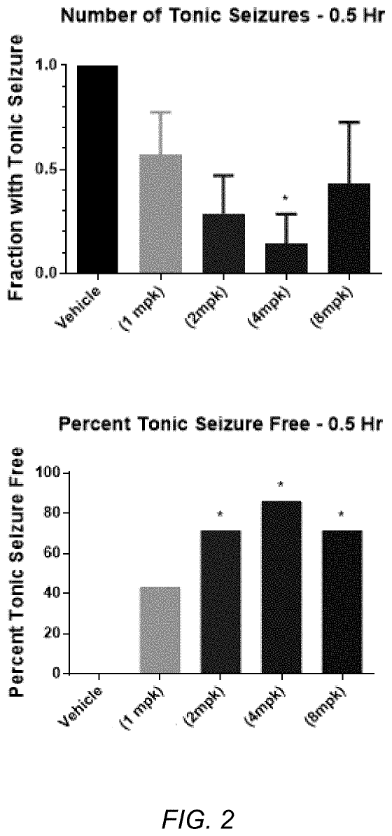

Compound A Dose Response: The dose response following oral administration of 1, 2, 4, or 8 mg/kg Compound A was evaluated in the mouse MES assay at 2 different time points: 0.5 and 2 hr after oral dosing (FIG. 1 and FIG. 2). Animals were randomly assigned to vehicle (n=7 at 2 hr, n=5 at 0.5 hr) or different dose groups (n=7 per dose, per time point) and the MES assay was performed by an experimenter blinded to the treatment conditions.

At the 2 hour time point, Compound A showed dose-dependent reduction in number of tonic seizures assessed by hindlimb extension. A dose of 8 mg/kg provided significant protection compared to vehicle treated mice (number of mice with tonic seizures/total number tested: 1 mg/kg: 5/7 (p=0.939), 2 mg/kg: 4/7 (p=0.598), 4 mg/kg: 2/7 (p=0.085), 8 mg/kg: 1/7 (p=0.023), vehicle: 6/7.—p values calculated by one-way ANOVA using Dunnett's multiple comparisons test). The same data was used to calculate the percentage of mice protected from hindlimb tonic extension (1 mg/kg: 28.6% (p>0.999), 2 mg/kg: 42.9% (p>0.999), 4 mg/kg: 71.4% (p=0.103), 8 mg/kg: 85.7% (p=0.029), vehicle: 14.3%; p values calculated by Fisher's exact test). Brain and plasma samples were collected from mice immediately after efficacy testing and analyzed using UHPLC-ESI-MS/MS (Table 1).

TABLE 1
Plasma and Brain Concentrations of Compound A

| | | | | Time of | | |
|---|---|---|---|---|---|---|
| | | | | Tissue | | |
| | Dose | # of | # of | Collection | Concentration (μM) | |
| Cmpd ID | (mg/kg) | Days | Doses | (hr) | Plasma | Brain |
| Cmpd A | 1 | 1 | 1 | 2 | 0.10 | 0.19 |
| Cmpd A | 2 | 1 | 1 | 2 | 0.13 | 0.21 |
| Cmpd A | 4 | 1 | 1 | 2 | 0.23 | 0.36 |
| Cmpd A | 8 | 1 | 1 | 2 | 0.43 | 0.56 |

Total brain and plasma concentrations of 1 mg/kg: 0.19 μM (70.2 ng/g) and 0.1 μM (35.8 ng/mL), 2 mg/kg: 0.21 μM (75.8 ng/g) and 0.13 μM (46.7 ng/mL), 4 mg/kg: 0.36 μM (131.3 ng/g) and 0.23 μM (83 ng/mL), 8 mg/kg: 0.56 μM (207.7 ng/g) and 0.43 μM (157.8 ng/mL), respectively.

At the 0.5 hour time point, doses of 1, 2 and 4 mg/kg showed dose dependent reduction in number of tonic seizures with extension of hindlimbs, with 4 mg/kg providing significant protection compared to vehicle treated mice (1 mg/kg: 4/7 (p=0.449), 2 mg/kg: 2/7 (p=0.088), 4 mg/kg: 1/7 (p=0.032), 8 mg/kg: 3/7 (p=0.215), vehicle: 5/5; one-way ANOVA calculated using Dunnett's multiple comparisons test. At 8 mg/kg, 1/7 animals had two tonic seizures with hindlimb extension after the stimulation, an occurrence that was not otherwise seen in other dose groups or vehicle treated mice. Similar dose dependent efficacy was observed for mice protected from hindlimb tonic extensor component of the tonic seizure (1 mg/kg: 42.9% (p>0.205), 2 mg/kg: 71.4% (p=0.028), 4 mg/kg: 85.7% (p=0.015), 8 mg/kg: 71.4% (p=0.028), vehicle: 0%; calculated using Fisher's exact test). Brain and plasma samples were collected from mice immediately after efficacy testing and analyzed using UHPLC-ESI-MS/MS (Table 2).

TABLE 2
Plasma and Brain Concentrations of Compound A

| | | | | Time of | | |
|---|---|---|---|---|---|---|
| | | | | Tissue | | |
| | Dose | # of | # of | Collection | Concentration (μM) | |
| Cmpd ID | (mg/kg) | Days | Doses | (hr) | Plasma | Brain |
| Cmpd A | 1 | 1 | 1 | 0.5 | 0.13 | 0.27 |
| Cmpd A | 2 | 1 | 1 | 0.5 | 0.22 | 0.36 |
| Cmpd A | 4 | 1 | 1 | 0.5 | 0.42 | 0.64 |
| Cmpd A | 8 | 1 | 1 | 0.5 | 0.71 | 1.09 |

The mean total brain and plasma concentrations for each dose group were: 1 mg/kg: 0.27 μM (97.9 ng/g) and 0.13 μM (48.9 ng/mL), 2 mg/kg: 0.36 μM (132 ng/g) and 0.22 μM (80.7 ng/mL), 4 mg/kg: 0.64 μM (234.7 ng/g) and 0.42 μM (154.4 ng/mL), 8 mg/kg: 1.09 μM (402 ng/g) and 0.71 μM (261.3 ng/mL), respectively.

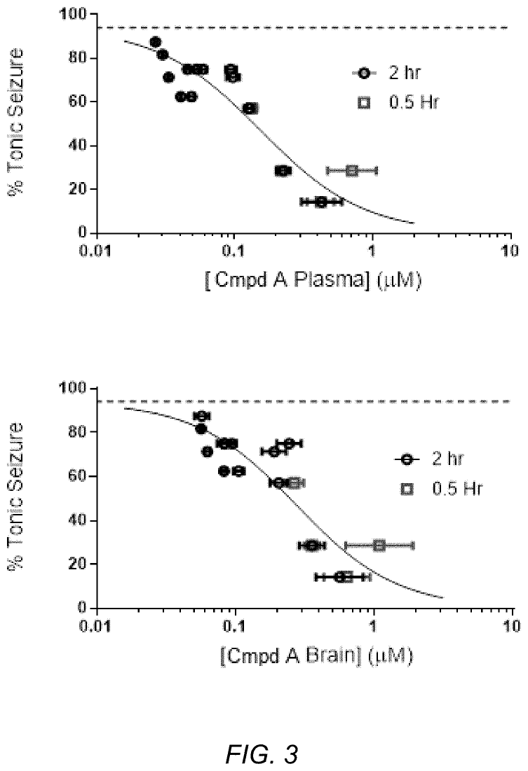

FIG. 3 shows the concentration-response relationship that summarizes all of the PK-PD data for Compound A in the MES assay. The upper panel shows the inhibition of tonic seizures as a function of plasma concentration and the lower panel shows the inhibition as a function of brain concentration. Although higher brain and plasma exposures were observed at 0.5 hour compared with 2 hour pre-treatment times, efficacy observed at each exposure level reflected brain or plasma concentration regardless of time after dosing (FIG. 3). Due to the well-defined dose response with 2 hour pre-treatment of Compound A, this time point was chosen for further studies. The data collected 2 hours after dosing was fit to a concentration-response curve with only the IC₅₀ and Hill coefficient varying for optimal fit. The horizontal dashed line indicates the incidence of tonic seizures in vehicle: 94.1±0.03% (mean±S.E.M., n=85). This value was taken as the top of the curve and the bottom was constrained to 0 (complete inhibition). The IC₅₀ estimates based upon brain and plasma concentration were 275 nM and 154 nM, respectively.

5.1.2 Combination of Compound A and Valproic Acid

Doses of 1 mg/kg PO with pre-treatment of 2 hr for Compound A and 100 mg/kg IP with pre-treatment of 0.5 hr for Valproic Acid were chosen for the initial combination study. A second experiment was conducted to repeat the findings of first study and test additional doses of Valproic acid (30 and 56 mg/kg IP) in order to quantify the extent of additional efficacy produced by Compound A (1 mg/kg PO). Results of these two experiments combined are discussed below.

Animals were randomly assigned and dosed orally with Compound A or vehicle 2 hours prior to efficacy testing, followed by IP dosing of Valproic Acid or vehicle 0.5 hour prior to efficacy testing. Based on this experimental design, animals in single dose control groups or the vehicle control group also received two doses each: (Compound A+Vehicle, Vehicle+Valproic Acid, Compound A+Valproic Acid, or Vehicle+Vehicle).

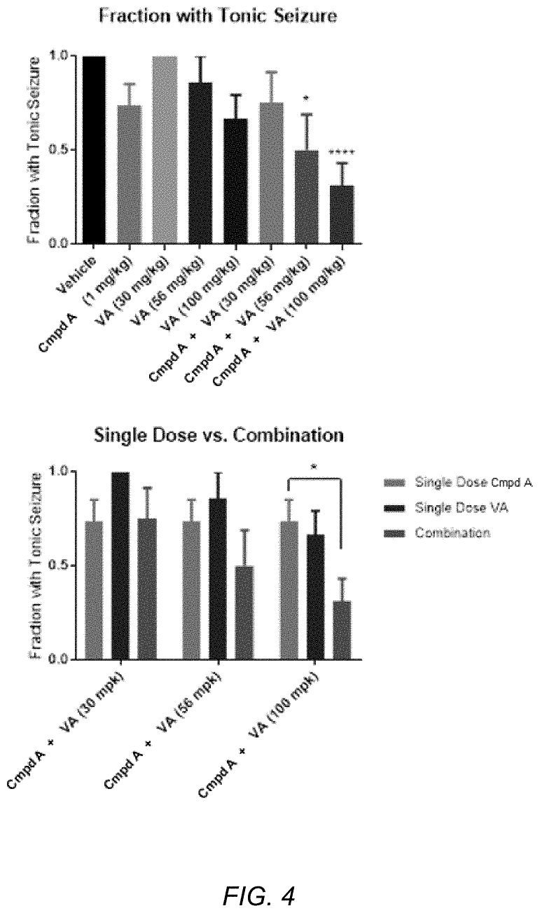

Co-dosing of Compound A and Valproic Acid at 100 mg/kg led to significant increase in number of mice without tonic seizures with extension of hindlimb when compared with Compound A single dose treated mice (Compound A+Valproic Acid at 100 mg/kg: 10/15 vs. Compound A at 1 mg/kg+Vehicle: 4/15 (p=0.021), and vs. Vehicle+Valproic Acid at 100 mg/kg: 5/15 (p=0.059); p values were calculated by two-way ANOVA followed by Dunnett's multiple comparisons test. A trend of increased protection against tonic seizures was noted with the combination dosing of Compound A and 56 mg/kg of Valproic Acid, while no difference was noted with the combination of Compound A and 30 mg/kg of Valproic Acid: Compound A+Valproic Acid at 56 mg/kg: 4/8 vs. Compound A at 1 mg/kg+Vehicle: 4/15 (p=0.396) and vs. Vehicle+Valproic Acid at 56 mg/kg: 1/7 (p=0.226); Compound A+Valproic Acid at 30 mg/kg: 2/8 vs. Compound A at 1 mg/kg+Vehicle: 4/15 (p=0.995) and vs. Vehicle+Valproic Acid at 56 mg/kg: 0/8 (p=0.463) (FIG. 4).

Both combination dose groups of Compound A (1 mg/kg)+Valproic Acid at 56 and 100 mg/kg led to significant protection when compared with vehicle treated animals: Compound A+Valproic Acid at 100 mg/kg: 10/15 (p=0.0001) and Compound A+Valproic Acid at 56 mg/kg: 4/8 (p=0.0381), vs. Vehicle+Vehicle: 0/15; p values were calculated by one-way ANOVA followed by Dunnett's multiple comparisons test.

Plasma and brain samples were collected from mice immediately after efficacy testing and analyzed using UHPLC-ESI-MS/MS (Table 3).

TABLE 3
Plasma and Brain Concentrations of Compound A and Valproic Acid

| | | +100 mg/kg Valproic | | 100 mg/kg Valproic | | | |
|---|---|---|---|---|---|---|---|
| 1 mg/kg Cmpd A | | Acid | | Acid | | +1 mg/kg Cmpd A | |
| Mean | SD | Mean | SD | Mean | SD | Mean | SD |

Plasma

| | | | | | | | |
|---|---|---|---|---|---|---|---|
| 24 ng/mL | 22 | 15 ng/mL | 6 | 151710 | 78134 | 144073 | 52101 |
| | | | | ng/mL | | ng/mL | |
| 0.066 μM | 0.05 | 0.042 μM | 0.016 | 1059 μM | 546 | 1006 μM | 364 |

Brain

| | | | | | | | |
|---|---|---|---|---|---|---|---|
| 59 ng/g | 51 | 36 ng/g | 23 | 40084 | 8722 | 42686 | 15028 |
| | | | | ng/g | | ng/g | |
| 0.16 μM | 0.14 | 0.097 μM | 0.062 | 280 μM | 61 | 298 μM | 105 |

There was no significant difference in plasma or brain concentrations of Compound A when dosed alone or co-dosed with Valproic Acid. The mean total plasma and brain concentrations of Compound A at 1 mg/kg when dosed alone: 0.066 μM (24 ng/mL) and 0.16 μM (59 ng/g), when dosed with Valproic Acid (30 mg/kg): 0.038 μM (14 ng/mL) and 0.075 μM (28 ng/g), when dosed with Valproic Acid (56 mg/kg): 0.050 μM (18 ng/mL) and 0.117 μM (43 ng/g), and when dosed with Valproic Acid (100 mg/kg): 0.042 μM (15 ng/mL) and 0.097 μM (36 ng/g). Likewise, there were no significant changes in plasma or brain concentrations of Valproic Acid at the 3 doses tested when combined with Compound A.

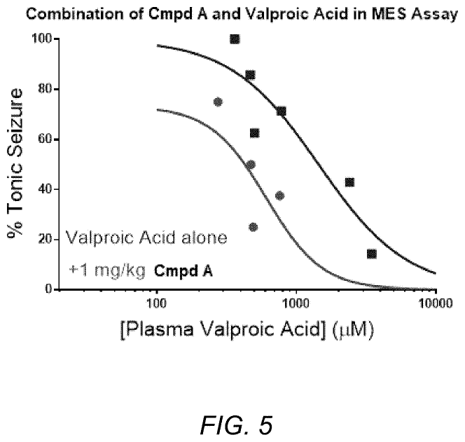

The co-dosing experiments with Compound A and VA were also analyzed as the effect of Compound A on the concentration-response relationship of VA to quantify the effect on the IC₅₀ for inhibition of tonic seizures by VA (FIG. 5).

5.1.3 Combination of Compound A and Levetiracetam

Levetiracetam is reported to be inactive in the MES assay (ED₅₀>500 mg/kg) but active in the 6 Hz, 32 mAmp assay with ED₅₀=19.4 mg/kg (Barton et al., Epilepsy Res. 2001, 47:217-227). High doses of Levetiracetam were chosen that are known to have biological activity.

Levetiracetam was evaluated in the MES assay by IP dosing of 120 and 150 mg/kg. These doses resulted in plasma concentrations of 1001 μM (170.3 μg/ml) and 1154 μM (196.4 μg/mL), and brain concentrations of 583 μM (99.2 μg/g) and 540 μM (91.9 μg/g) respectively (Table 4).

TABLE 4
Plasma and Brain Concentrations of Compound A and Levetiracetam

| | | +120 mg/kg | | 120 mg/kg | | | |
|---|---|---|---|---|---|---|---|
| 1 mg/kg Cmpd A | | Levetiracetam | | Levetiracetam | | +1 mg/kg Cmpd A | |
| Mean | SD | Mean | SD | Mean | SD | Mean | SD |

Plasma

| | | | | | | | |
|---|---|---|---|---|---|---|---|
| 11.1 ng/mL | 3.8 | 11.1 ng/mL | 5.6 | 170.3 μg/ml | 12.7 | 162.8 μg/ml | 12.8 |
| 0.03 μM | 0.01 | 0.03 μM | 0.02 | 1001 μM | 75 | 957 μM | 75 |

Brain

| 20.9 ng/g | 4.8 | 20.0 ng/g | 12.5 | 99.2 μg/g | 14.4 | 94.3 μg/g | 2.0 |
|---|---|---|---|---|---|---|---|
| 0.06 μM | 0.01 | 0.05 μM | 0.03 | 583 μM | 85 | 554 μM | 12 |
| | | +150 mg/kg | | 150 mg/kg | | | |
| 1.5 mg/kg Cmpd A | | Levetiracetam | | Levetiracetam | | +1.5 mg/kg Cmpd A | |
| Mean | SD | Mean | SD | Mean | SD | Mean | SD |

Plasma

| | | | | | | | |
|---|---|---|---|---|---|---|---|
| 15 ng/mL | 4 | 25 ng/mL | 24 | 196.4 μg/mL | 29.7 | 210.3 μg/mL | 28.9 |
| 0.04 μM | 0.01 | 0.07 μM | 0.06 | 1154 μM | 174 | 1235 μM | 169 |

Brain

| | | | | | | | |
|---|---|---|---|---|---|---|---|
| 31 ng/g | 8 | 52 ng/g | 56 | 91.9 μg/g | 7.0 | 90.4 μg/g | 12.8 |
| 0.08 μM | 0.02 | 0.14 μM | 0.15 | 539.9 μM | 41 | 531 μM | 75.3 |

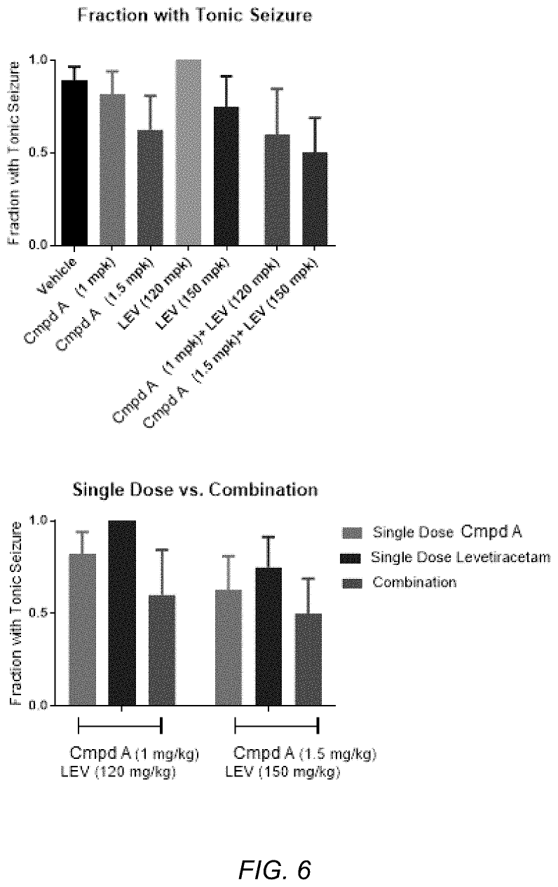

Neither dose produced a significant increase in the fraction of mice without tonic seizures compared to vehicle treated mice: 120 mg/kg: 0/6 (p=0.98), 150 mg/kg 2/8 (p=0.93), vehicle: 2/18; p value calculated by one-way ANOVA by Dunnett's multiple comparisons test (FIG. 6).

Co-dosing with 1 or 1.5 mg/kg Compound A had no effect on plasma or brain levels of Levetiracetam: 957 μM (163 μg/mL) plasma and 554 μM (943 μg/g) brain (LEV 120 mg/kg+1 mg/kg Compound A); 1235 μM (210 μg/mL) plasma and 531 μM (90.4 μg/g) brain (LEV 150 mg/kg+1.5 mg/kg Compound A). Exposure values for Compound A at 1 mg/kg alone or co-dosed with Levetiracetam at 120 mg/kg were 0.03 μM (11.1 ng/mL) and 0.03 μM (11.1 ng/mL) in plasma, and 0.06 μM (20.9 ng/g) and 0.05 μM (20.0 ng/g) in brain, respectively. Exposure values for Compound A at 1.5 mg/kg alone or co-dosed with Levetiracetam at 150 mg/kg were 0.04 μM (15 ng/mL) and 0.07 μM (25 ng/mL) in plasma, and 0.08 μM (31 ng/g) and 0.14 μM (52 ng/g) in brain, respectively.

The increase in fraction of mice without tonic seizures at either dose combination was not significantly greater than the effect of Compound A alone: Compound A at 1 mg/kg: 2/11 vs. Compound A (1 mg/kg)+LEV (120 mg/kg): 2/5, p=0.578; and Compound A at 1.5 mg/kg: 3/8 vs. Compound A (1.5 mg/kg)+LEV (150 mg/kg): 4/8, p=0.805; p value calculated by two-way ANOVA by Dunnett's multiple comparisons test (FIG. 6).

5.1.4 Combination of Compound A and Phenytoin

Kv7 channels co-localize in the axon initial segment with Naᵥ1.6 and Naᵥ1.2 channels. Since Compound A activates Kᵥ7 channels and Phenytoin inhibits voltage gated sodium channels, a favorable interaction was anticipated, but the magnitude was unknown.

Dose response of Compound A (0.25, 0.75, 1, 1.5, and 2.5 mg/kg) administered PO with 2 hours pre-treatment time was tested in the MES assay in presence or absence of a pre-determined constant dose of Phenytoin (2 mg/kg, IP, 2 hr pre-treatment).

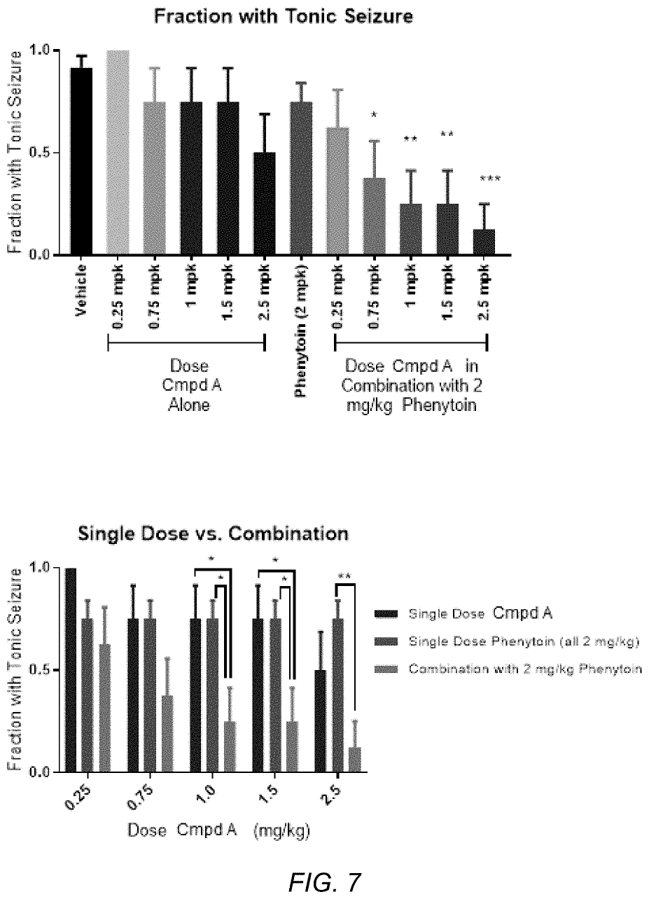

Compared to vehicle treated animals, single doses of Compound A led to dose dependent increase in fraction of animals without tonic seizures: Compound A at 0.25 mg/kg: 0/8 (p=0.999), 0.75 mg/kg: 2/8 (p=0.948), 1 mg/kg: 2/8 (p=0.948), 1.5 mg/kg: 2/8 (p=0.948), 2.5 mg/kg: 4/8 (p=0.12); Phenytoin at 2 mg/kg: 6/24 (p=0.72); Vehicle 2/24. When co-dosed with Phenytoin, Compound A at doses of 0.75, 1, 1.5 and 2.5 mg/kg led to significant increase in the fraction of animals without tonic seizures when compared with vehicle treated animals: Compound A 0.25 mg/kg+Phenytoin 2 mg/kg: 3/8 (p=0.48), Compound A 0.75 mg/kg+Phenytoin 2 mg/kg: 5/8 (p=0.018), Compound A 1 mg/kg+Phenytoin 2 mg/kg: 6/8 (p=0.002), Compound A 1.5 mg/kg+Phenytoin 2 mg/kg: 6/8 (p=0.002), Compound A 2.5 mg/kg+Phenytoin 2 mg/kg: 7/8 (p=0.0001); p values were calculated by one-way ANOVA Dunnett's multiple comparisons test (FIG. 7).

Comparison of animals administered with single dose of Compound A with the groups co-dosed with both Compound A and Phenytoin, showed significant increase in fraction of animals without tonic seizures in co-dose groups at doses 1 and 1.5 mg/kg. A similar trend was observed when comparing a single dose of Phenytoin with co-dose groups, with significant seizure freedom noted at 1, 1.5 and 2.5 mg/kg of Compound A: Compound A 0.25+Phenytoin 2 mg/kg combination: 3/8 vs. single dose Compound A (0.25 mg/kg): 0/8 (p=0.165) and 6/24 Phenytoin (2 mg/kg; p=0.715), Compound A 0.75+Phenytoin 2 mg/kg combination: 5/8 vs. single dose Compound A (0.75 mg/kg): 2/8 (p=0.165) and 6/24 Phenytoin (2 mg/kg; p=0.074), Compound A 1+Phenytoin 2 mg/kg combination: 6/8 vs. single dose Compound A (1 mg/kg): 2/8 (p=0.048) and 6/24 Phenytoin (2 mg/kg; p=0.012), Compound A 1.5+Phenytoin 2 mg/kg combination: 6/8 vs. single dose Compound A (1.5 mg/kg): 2/8 (p=0.048) and 6/24 (Phenytoin at 2 mg/kg; p=0.012), Compound A 2.5+Phenytoin 2 mg/kg combination: 7/8 vs. single dose Compound A (2.5 mg/kg): 4/8 (p=0.165) and 6/24 Phenytoin (2 mg/kg; p=0.001); p values were calculated by two-way ANOVA followed by Dunnett's multiple comparisons test (FIG. 7).

No Significant change in plasma or brain concentrations was noted for Phenytoin when dosed with or without Compound A (Table 4).

TABLE 4
Plasma and Brain Concentration of Compound A and Phenytoin

| 2 mg/kg | | +0.25 mg/kg | | +0.75 mg/kg | | +1 mg/kg | | +1.5 mg/kg | | +2.5 mg/kg | |
|---|---|---|---|---|---|---|---|---|---|---|---|
| Phenytoin | | Cmpd A | | Cmpd A | | Cmpd A | | Cmpd A | | Cmpd A | |
| Mean | SD | Mean | SD | Mean | SD | Mean | SD | Mean | SD | Mean | SD |

Plasma

| | | | | | | | | | | | |
|---|---|---|---|---|---|---|---|---|---|---|---|
| 1019 | 206 | 1014 | 80 | 1025 | 149 | 989 | 101 | 1210 | 232 | 971 | 82 |
| ng/mL | | ng/mL | | ng/mL | | ng/mL | | ng/mL | | ng/mL | |
| 4.1 μM | 0.8 | 4.0 μM | 0.3 | 4.1 μM | 0.6 | 3.9 μM | 0.4 | 4.8 μM | 0.9 | 3.9 μM | 0.3 |

Brain

| | | | | | | | | | | | |
|---|---|---|---|---|---|---|---|---|---|---|---|
| 1052 | 137 | 1055 | 126 | 1070 | 119 | 1263 | 67 | 1196 | 133 | 1051 | 67 |
| ng/g | | ng/g | | ng/g | | ng/g | | ng/g | | ng/g | |
| 4.2 μM | 0.5 | 4.2 μM | 0.5 | 2.9 μM | 0.3 | 5.0 μM | 0.4 | 4.8 μM | 0.5 | 2.9 μM | 0.2 |

Plasma and brain exposure of Phenytoin 2 mg/kg single dose respectively: 4.1 μM (1019 ng/mL) and 4.2 μM (1052 ng/g), combined with 0.25 mg/kg of Compound A: 4.0 μM (1014 ng/mL) and 4.2 μM (1055 ng/g), combined with 0.75 mg/kg of Compound A: 4.1 μM (1025 ng/mL) and 2.9 μM (1070 ng/g), combined with 1 mg/kg of Compound A: 3.9 μM (989 ng/mL) and 5.0 μM (1263 ng/g), combined with 1.5 mg/kg of Compound A: 4.8 μM (1210 ng/mL) and 4.8 μM (1196 ng/g), and combined with 2.5 mg/kg of Compound A: 3.9 μM (971 ng/mL) and 2.9 μM (1051 ng/g).

Similarly, there are no significant changes in plasma and brain concentration for Compound A when dosed alone or in combination with Phenytoin (Table 5).

TABLE 5
Plasma and Brain Concentration of Compound A and Phenytoin
Plasma

| 0.25 mg/kg | | +2 mg/kg | | 0.75 mg/kg | | +2 mg/kg | | 1 mg/kg | | +2 mg/kg | |
|---|---|---|---|---|---|---|---|---|---|---|---|
| Cmpd A | | Phenytoin | | Cmpd A | | Phenytoin | | Cmpd A | | Phenytoin | |
| Mean | SD | Mean | SD | Mean | SD | Mean | SD | Mean | SD | Mean | SD |
| 4.3 | 2.6 | 5.6 | 1.3 | 11.7 | 2.3 | 16.0 | 3.9 | 17.0 | 8.0 | 19.0 | 5.0 |
| ng/mL | | ng/mL | | ng/mL | | ng/mL | | ng/mL | | ng/mL | |
| 0.01 | 0.01 | 0.02 | 0.00 | 0.03 | 0.01 | 0.04 | 0.01 | 0.05 | 0.02 | 0.05 | 0.01 |
| μM | | μM | | μM | | μM | | μM | | μM | |

| 1.5 mg/kg | | +2 mg/kg | | 2.5 mg/kg | | +2 mg/kg | |
|---|---|---|---|---|---|---|---|
| Cmpd A | | Phenytoin | | Cmpd A | | Phenytoin | |
| Mean | SD | Mean | SD | Mean | SD | Mean | SD |
| 19.5 | 12.5 | 23.4 | 12.8 | 45.1 ng/mL | 12.7 | 58.4 ng/mL | 14.6 |
| ng/mL | | ng/mL | | | | | |
| 0.05 | 0.03 | 0.06 | 0.04 | 0.12 μM | 0.03 | 0.16 μM | 0.04 |
| μM | | μM | | | | | |

Brain

| 0.25 mg/kg | | +2 mg/kg | | 0.75 mg/kg | | +2 mg/kg | | 1 mg/kg | | +2 mg/kg | |
|---|---|---|---|---|---|---|---|---|---|---|---|
| Cmpd A | | Phenytoin | | Cmpd A | | Phenytoin | | Cmpd A | | Phenytoin | |
| Mean | SD | Mean | SD | Mean | SD | Mean | SD | Mean | SD | Mean | SD |
| 14.8 | 2.6 | 18.4 | 3.3 | 43.8 | 7.6 | 56.7 | 17.3 | 35.0 | 17 | 37.0 | 11.0 |
| ng/g | | ng/g | | ng/g | | ng/g | | ng/g | | ng/g | |
| 0.04 | 0.01 | 0.05 | 0.01 | 0.12 | 0.02 | 0.15 | 0.05 | 0.09 | 0.04 | 0.10 | 0.03 |
| μM | | μM | | μM | | μM | | μM | | μM | |

| 1.5 mg/kg | | +2 mg/kg | | 2.5 mg/kg | | +2 mg/kg | |
|---|---|---|---|---|---|---|---|
| Cmpd A | | Phenytoin | | Cmpd A | | Phenytoin | |
| Mean | SD | Mean | SD | Mean | SD | Mean | SD |
| 30.8 | 14.3 | 42.0 | 18.9 | 144.1 ng/g | 8.2 | 186.5 ng/g | 38 |
| ng/g | | ng/g | | | | | |
| 0.08 | 0.04 | 0.11 | 0.05 | 0.39 μM | 0.07 | 0.51 μM | 0.10 |
| μM | | μM | | | | | |

Compound A at 0.25 mg/kg single dose plasma and brain vs. in combination with Phenytoin (2 mg/kg) respectively: 0.01 μM (4.3 ng/mL) and 0.04 μM (14.8 ng/g) vs. 0.02 μM (5.6 ng/mL) and 0.05 μM (18.4 ng/g), 0.75 mg/kg single dose vs. combination: 0.03 μM (11.7 ng/mL) and 0.12 μM (43.8 ng/g) vs. 0.04 μM (16.0 ng/mL) and 0.15 μM (56.7 ng/g), 1 mg/kg single dose vs. combination: 0.05 μM (17.0 ng/mL) and 0.10 μM (35.0 ng/g) vs. 0.05 μM (19.0 ng/mL) and 0.10 μM (37.0 ng/g), 1.5 mg/kg single dose vs. combination: 0.05 μM (19.5 ng/mL) and 0.08 μM (30.8 ng/g) vs. 0.06 μM (23.4 ng/mL) and 0.11 μM (42 ng/g), 2.5 mg/kg single dose vs. combination: 0.12 μM (45.1 ng/mL) and 0.39 μM (144.1 ng/g) vs. 0.16 μM (58.4 ng/mL) and 0.51 μM (186.5 ng/g).

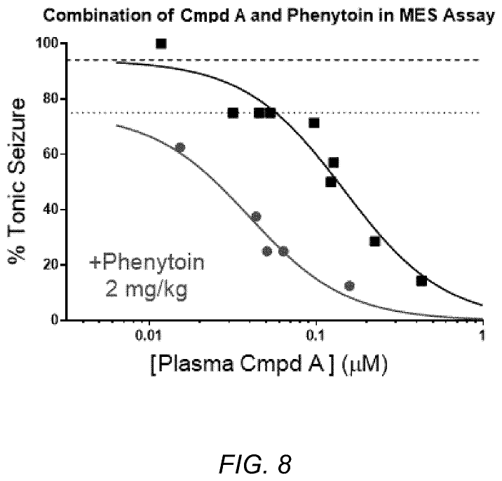

The co-dosing experiments with Compound A and phenytoin were also analyzed as the effect of phenytoin on the concentration-response relationship of Compound A to quantify the effect on the IC₅₀ for inhibition of tonic seizures by Compound A (FIG. 8).

5.1.5 Conclusions

Compound A demonstrated dose-dependent efficacy in the mouse MES assay that correlated well with plasma and brain exposure values. Combination of Compound A and Valproic Acid at 100 mg/kg or Phenytoin at 2 mg/kg led to higher efficacy observed in the MES assay than observed with either compound alone. Co-dosing of Compound A with Levetiracetam did not provide significant improvement in efficacy in the MES assay when compared with Compound A alone.

Valproic acid (30, 56 or 100 mg/kg IP, dosed 0.5 hours before MES): Combining Compound A (1 mg/kg PO, 2 hours before MES) with Valproic Acid (VA) dosed at 100 mg/kg produced greater inhibition than when Compound A was dosed alone (Compound A at 1 mg/kg+Valproic Acid at 100 mg/kg: 10/15 vs. Compound A at 1 mg/kg+Vehicle: 4/15 (p=0.021)) (Single dose groups: n=15 Compound A at 1 mg/kg, n=15 Valproic Acid at 100 mg/kg, n=7 Valproic Acid at 30 and 56 mg/kg; Combination dose groups: n=15 Compound A 1 mg/kg+Valproic Acid 100 mg/kg, n=8 Compound A+Valproic Acid 30 and 56 mg/kg; n=15 vehicle). Compound A dosed at 1 mg/kg reduced the plasma level of Valproic Acid needed for protection against tonic seizures in the MES assay. The IC₅₀ for Valproic Acid alone was 1440 μM. When combined with 1 mg/kg Compound A, the IC₅₀ for Valproic Acid was 608 μM, a decrease of 2.37 fold. The mean total plasma concentration of Compound A at 1 mg/kg when dosed alone: 0.07 μM (24.2 ng/mL) and when dosed with Valproic Acid (30 mg/kg): 0.04 μM (14.0 ng/mL), when dosed with Valproic Acid (56 mg/kg): 0.03 μM (12.0 ng/mL), and when dosed with Valproic Acid (100 mg/kg): 0.04 μM (15.0 ng/mL).

Levetiracetam (120 and 150 mg/kg IP, dosed 2 hours before MES): A dose of 1 mg/kg of Compound A produced a plasma concentration of 66±14 nM (mean±SEM, n=15) and reduced the fraction with tonic seizures by 26.7%. Co-dosing of Compound A with Levetiracetam (LEV) did not provide improvement in protection against tonic seizures when compared with Compound A alone: Compound A at 1 mg/kg: 2/11 vs. Compound A (1 mg/kg)+LEV (120 mg/kg): 2/5, p=0.578; and Compound A at 1.5 mg/kg: 3/8 vs. Compound A (1.5 mg/kg)+LEV (150 mg/kg): 4/8, p=0.805. Plasma Concentrations for Compound A at 1 mg/kg alone or co-dosed with LEV at 120 mg/kg were 0.03 μM (11.1 ng/mL) and 0.03 μM (11.1 ng/mL), at 1.5 mg/kg alone or co-dosed with LEV at 150 mg/kg were 0.041 μM (15 ng/mL) and 0.068 μM (25 ng/mL) respectively.

Phenytoin (2 mg/kg IP, dosed 2 hours before MES): Combining Compound A with Phenytoin dosed at 2 mg/kg produced greater inhibition than when Compound A was dosed alone. When co-dosed 2 hours before MES with Phenytoin, Compound A at oral doses of 0.75, 1, 1.5 and 2.5 mg/kg led to significant increase in the fraction of mice without tonic seizures when compared with vehicle treated animals: Compound A 0.25 mg/kg+Phenytoin 2 mg/kg: 3/8 (p=0.48), Compound A 0.75 mg/kg+Phenytoin 2 mg/kg: 5/8 (p=0.013), Compound A 1 mg/kg+Phenytoin 2 mg/kg: 6/8 (p=0.002), Compound A 1.5 mg/kg+Phenytoin 2 mg/kg: 6/8 (p=0.002), Compound A 2.5 mg/kg+Phenytoin 2 mg/kg: 7/8 (p=0.0001). The IC₅₀ for Compound A alone was 147 nM. When combined with 2 mg/kg phenytoin, the IC₅₀ for Compound A was 39.7 nM. There were no significant changes in plasma concentration for Compound A when dosed alone or in combination with Phenytoin: Compound A at 0.25 mg/kg single dose vs. in combination with Phenytoin (2 mg/kg): 0.01 μM (4.3 ng/mL) vs. 0.02 μM (5.6 ng/mL), 0.75 mg/kg single dose vs. combination: 0.03 μM (11.7 ng/mL) vs. 0.04 μM (16 ng/mL), 1 mg/kg single dose vs. combination: 0.05 μM (17 ng/mL) vs. 0.05 μM (19 ng/mL), 1.5 mg/kg single dose vs. combination: 0.05 μM (19.5 ng/mL) vs. 0.06 μM (23.4 ng/mL), 2.5 mg/kg single dose vs. combination: 0.12 μM (45.1 ng/mL) vs. 0.16 μM (58.4 ng/mL).

### 5.2. Example 2. Anticonvulsant Effects of Compound A Alone and in Combination with Lacosamide

The efficacy of Compound A and its pharmacological interaction with Lacosamide was evaluated after oral dosing in the mouse maximal electroshock seizure (AC-MES) assay.

The objective of these studies was to characterize the dose-dependent anticonvulsant activity of Compound A and its pharmacological interaction with Lacosamide in the AC-MES assay in mice after a single oral administration of the compounds. The AC-MES assay is typically responsive to non-selective sodium channel blockers and potassium channel openers, and has been used as a translational animal model for partial onset seizures. The MES assay has been used extensively for the screening and characterization of novel anti-seizure compounds (Löscher et al., Epilepsy Res. 1991, 8(2):79-94; Piredda et al., J Pharmacol Exp Ther. 1985, 232(3):741-745; and White et al., Ital J Neurol Sci. 1995, 16(1-2):73-77). Following electroshock stimulation at sufficiently high currents, mice and rats exhibit a tonic extension followed by hindlimb clonus. If a test compound is able to prevent tonic extension it was considered protective.

The anticonvulsant efficacy of Compound A and Lacosamide, and the effect of combining Compound A with Lacosamide were tested in the AC-MES assay in male CF-1 mice following a single PO dose. Plasma and brain samples were obtained to understand the relationship between drug concentration and efficacy.

5.2.1 Materials and Methods

Test Compound—Compound A

| | |
|---|---|
| Identity: | Compound A |
| Batch Number: | Batch 12 |
| Physical Description: | Solid, white powder |
| Purity: | 98.8% |
| Supplier: | Xenon Pharmaceuticals Inc. |

Test Compound—Lacosamide

| | |
|---|---|
| Identity: | Lacosamide |
| Batch Number: | Batch 03 |
| Physical Description: | White powder |
| Purity: | >95% |
| Supplier: | Toronto Research Chemicals |

Vehicle F1: 0.5% methyl cellulose and 0.2% Tween-80 in deionized (DI) water. 0.8 L of DI water was heated up to 70° C. to 80° C. Five grams of methyl cellulose was weighed and slowly added in small portions to heated DI water. The mixture was stirred until it formed a homogeneous milky suspension. The suspension was moved to a cold room and stirred overnight to get a clear solution. Two mL of Tween-80 was added to the clear solution and diluted up to 1 L with DI water. The vehicle solution was stored at 2° C. to 8° C.

Vehicle F2: 5% dimethyl sulfoxide (DMSO) and F1. 5% DMSO was added to F1 vehicle.

Dose Formulation: Compound A and Lacosamide were weighed into separate vials.

Compound A was formulated in F2 vehicle and Lacosamide was formulated in F1 vehicle. An appropriate amount of vehicle was added to the Compound A and Lacosamide powders then mixed on the IKA T-18 Ultra-Turrax Homogenizer to create a uniform suspension at the desired concentration. The vials were then wrapped in aluminum foil to protect from light, and placed on a stir plate until the time of dosing.

Test System

| | |
|---|---|
| Species/Strain: | CF-1 mouse |
| Number and Sex: | 144 male |
| Source: | Charles River Laboratories |
| Age and Body Weight | Animals were 6.5 to 8.5 weeks old and body weight ranged from |
| at Initiation of Dosing: | 27 to 38 g at initiation of dosing. |
| Housing: | Animals were group-housed (4 per cage) in cages that are in |
| | accordance with applicable animal welfare laws and regulations. |
| | Temperature was between 18° C. and 25° C. and relative humidity |
| | between 45% and 65%. |
| Acclimation: | Upon arrival the animals were examined to assure satisfactory |
| | health status and acclimated to the facility for at least 5 days |
| | before study. |
| Food/Water: | Animals were provided Certified Rodent Chow (Teklad Rodent |
| | Chow #2014) ad libitum. |
| | Tap water was available ad libitum. |

Experimental Design: The animals were assigned to treatment groups as indicated in Table 6, Table 7, Table 8, and Table 9.

Three studies were conducted with Compound A and four studies were conducted with Lacosamide. Their respective study dates are shown in Table 6 to Table 9.

TABLE 6
Experimental Groups - Study 2A

| | | | | | Pre- | | |
|---|---|---|---|---|---|---|---|
| | | | | | Treat- | | |
| | | | | | ment | Dose | |
| | | Dose | | Number | Time | Volume | Formu- |
| Group | Compound | (mg/kg) | Route | of Mice | (h) | (mL/kg) | lation |
| 1 | Vehicle | 0 | PO | 8 | 0.5 | 10 | F2 |
| 2 | Compound A | 1 | PO | 8 | 0.5 | 10 | F2 |
| 3 | Compound A | 5 | PO | 8 | 0.5 | 10 | F2 |
| 4 | Compound A | 10 | PO | 8 | 0.5 | 10 | F2 |
| 5 | Lacosamide | 6 | PO | 8 | 2 | 10 | F1 |
| 6 | Lacosamide | 8 | PO | 8 | 2 | 10 | F1 |

PO: per os, oral.

TABLE 7
Experimental Groups - Study 2B

| | | | | | Pre- | | |
|---|---|---|---|---|---|---|---|
| | | | | | Treat- | | |
| | | | | | ment | Dose | |
| | | Dose | | Number | Time | Volume | Formu- |
| Group | Compound | (mg/kg) | Route | of Mice | (h) | (mL/kg) | lation |
| 1 | Vehicle | 0 | PO | 8 | 0.5 | 10 | F2 |
| 2 | Compound A | 5 | PO | 8 | 0.5 | 10 | F2 |
| 3 | Compound A | 7.5 | PO | 7 | 0.5 | 10 | F2 |
| 4 | Compound A | 10 | PO | 9 | 0.5 | 10 | F02 |
| 5 | Lacosamide | 10 | PO | 8 | 2 | 10 | F1 |

PO: per os, oral.

TABLE 8
Experimental Groups - Study 2C

| | | | | | Pre- | | |
|---|---|---|---|---|---|---|---|
| | | Dose | | Number | Treatment | Dose Volume | |
| Group | Compound | (mg/kg) | Route | of Mice | Time (h) | (mL/kg) | Formulation |
| 1 | Vehicle | 0 | PO | 8 | 0.5 | 10 | F2 + F1 |
| 2 | Compound A | 3 | PO | 8 | 0.5 | 10 | F2 + F1 |
| 3 | Lacosamide | 10 | PO | 8 | 2 | 10 | F2 + F1 |
| 4 | Compound A + | 3 + 10 | PO | 8 | 0.5 + 2 | 10 | F2 + F1 |
| | Lacosamide | | | | | | |

PO: per os, oral.

TABLE 9
Experimental Groups - Study 2D

| | | | | | Pre- | | |
|---|---|---|---|---|---|---|---|
| | | | | | Treat- | | |
| | | | | | ment | Dose | |
| | | Dose | | Number | Time | Volume | Formu- |
| Group | Compound | (mg/kg) | Route | of Mice | (h) | (mL/kg) | lation |
| 1 | Vehicle | 0 | PO | 8 | 2 | 10 | F1 |
| 2 | Lacosamide | 6 | PO | 8 | 2 | 10 | F1 |
| 3 | Lacosamide | 20 | PO | 8 | 2 | 10 | F1 |

PO: per os, oral.

Blinding and Randomization: The experimenter administering the compound assigned each animal randomly to a treatment group. A different experimenter, blinded to treatment group allocation, performed the test. In addition, compound dosing and MES testing were done in different rooms to make sure that an experimenter performing the test is completely blinded to treatment. All animals tested in a given experiment had an equal chance of assignment to any treatment group.

Clinical Observations: All animals were observed for abnormal behavior for 10 minutes after dosing with Compound A and Lacosamide by the experimenter who dosed the animals and again at the time of stimulation by the experimenter who tested the animals. Any qualitative changes from normal behavior were recorded.

AC-MES Assay: The MES test has been extensively used in the search for anticonvulsant substances (Piredda et al., Löscher et al., and White et al.). The MES test is considered a model for generalized tonic-clonic (GTC) seizures and provides an indication of a compound's ability to prevent seizure spread. In the AC-MES model, an electroshock of alternating current (60 Hz, 40 mA) was delivered for 0.2 seconds by corneal electrodes (HSE-HA Rodent Shocker, Harvard Apparatus, model no: 73-0105). CF-1 mice were dosed PO (according to Standard Operating Procedure (SOP) TECH-006) with vehicle or Compound A 0.5 hours before the electroshock assay Immediately prior to the electroshock stimulation, the animals' eyes were anesthetized with a topical application of 0.5% Alcaine solution (proparacaine hydrochloride, one drop per eye). Mice were then restrained, the corneal electrodes applied, and the shock administered. In naïve animals, the seizure is characterized by an initial generalized tonic seizure with hindlimb tonic extensor component. An animal is considered protected from MES-induced seizures upon abolition of the hindlimb tonic extensor component of the seizure and is then scored “0”. If a mouse displays tonic hindlimb extension, the score is “1”. Mice were euthanized immediately after an initial seizure score assessment following the electroshock for plasma and brain collection.

Sample Collection and Preparation: Mice were anesthetized by isoflurane inhalation (according to SOP TECH-018) until they reached a surgical plane of anesthesia. Then a syringe (1 mL syringe with a 22 gauge needle) was inserted under the sternum into the heart (according to SOP TECH-031). Approximately 0.5 mL of blood was collected, deposited into a K₂EDTA tube, and stored on ice. Animals were then euthanized by cervical dislocation. Brains were removed, placed into a pre-weighed vial, and snap frozen on dry ice. At the end of the sample collection, blood was centrifuged at 4000 rpm for 10 minutes at 4° C., and the plasma pipetted into a labeled tube. All samples were stored in a −80° C. freezer until the time of bioanalysis.

Plasma Samples: Extraction of plasma samples was carried out by protein precipitation using acetonitrile. Diluted plasma samples (50 μL) were mixed with 50 μL of internal standard (IS) solution in 1:1 acetonitrile:water (v:v) followed by addition of 200 μL of acetonitrile. Samples were vortexed for 30 seconds, centrifuged at 13,000 rpm for 20 minutes, decanted into a 96-well plate and further centrifuged at 4000 rpm for 20 minutes. The samples were analyzed by ultra-high performance liquid chromatography electrospray ionization tandem mass spectrometry (UHPLC-ESI-MS/MS) as described in the bioanalysis procedure below.

Brain Samples: Prior to extraction, pre-weighed whole brains were homogenized in 1:1 acetonitrile:water (v:v) (2 mL per mouse brain) using an IKA T18 Ultra-Turrax Homogenizer at the setting of 4 for approximately 1 minute. The homogenate was centrifuged at 13,000 rpm for 20 minutes and 50 μL of the supernatant were treated exactly as described in above for plasma samples.

Bioanalysis Procedures: All samples, including plasma and brain homogenate extracts (including calibration standards and quality control (QC) samples prepared in K2EDTA mouse plasma), were extracted by protein precipitation. To each 50 μL aliquot of sample, 50 μL of IS solution (2500 ng/mL of Lacosamide in water:acetonitrile (1:1)) and 50 μL of 6% (v:v) phosphoric acid in water were added, then 200 μL acetonitrile was added. The samples in 1.7 mL tubes were vortex-mixed then centrifuged for 20 minutes at 13,000 rpm. Fifty microliters of the supernatant was mixed with 150 μL of water:acetonitrile (1:1) in a 96 well plate, which was centrifuged post-mixing for 20 minutes at 4000 rpm. The samples were then ready for UHPLC-MS/MS analysis.

Samples were analyzed for Compound A by a research-grade UHPLC-MS/MS method using the conditions listed below.

| Instrument: | Sciex TQ-5500 | |
|---|---|---|
| Matrix: | Mouse plasma (K₂EDTA) | |
| | Mouse brain homogenate | |
| Analyte: | Compound A | |
| Internal Standard (IS): | (S)-5-chloro-4-((1-(5-chloro-2- | |
| | fluorophenyl)ethyl)amino)-2-fluoro-N- | |
| | (pyrazin-2-yl)benzenesulfonamide | |
| MS Conditions: | ESI: positive | |
| | Compound A: [M + H]⁺ m/z 369.2/247.2 | |
| | (S)-5-chloro-4-((1-(5-chloro-2- | |
| | fluorophenyl)ethyl)amino)-2-fluoro-N- | |
| | (pyrazin-2-yl)benzenesulfonamide | |
| | (IS): [M + H]⁺ m/z 458.793/157 | |
| UHPLC Conditions: | Time (min) | % B |
| | 0.0 | 10 |
| | 0.2 | 10 |
| | 3.4 | 6 |
| | 3.5 | 100 |
| | 4.0 | 100 |
| | 4.1 | 10 |
| | 5.0 | 10 |

| | |
|---|---|
| | Mobile Phase A: 0.1% formic acid in water |
| | Mobile Phase B: 0.1% formic acid in acetonitrile |
| Calibration Standards | Standards were prepared at 2.34, 4.69, 9.38, |
| | 18.8, 37.5, 75.0, 150, 300, 600, 1200, 2400, |
| | and 4800 ng/mL |
| QC Samples | QC samples were prepared at 14.0, 225 and |
| | 3600 ng/mL, analyzed in triplicate. |

Samples were analyzed for Lacosamide by a research-grade UHPLC-MS/MS method using the conditions listed below.

| Instrument: | Sciex TQ-5500 | |
|---|---|---|
| Matrix: | Mouse plasma (K₂EDTA) | |
| | Mouse brain homogenate | |
| Analyte: | Lacosamide | |
| Internal Standard (IS): | (S)-5-((1-benzylpyrrolidin-3-yl)(methyl)amino)- | |
| | 6-methyl-N-(thiazol-4-yl)pyridine-2-sulfonamide | |
| MS Conditions: | ESI: positive | |
| | Lacosamide: [M + H]⁺ m/z 251.168/91 | |
| | (S)-5-((1-benzylpyrrolidin-3-yl)(methyl)amino)- | |
| | 6-methyl-N-(thiazol-4-yl)pyridine-2-sulfonamide | |
| | (IS): [M + H]⁺ m/z 551.1/214.0 | |
| UHPLC Conditions: | Time (min) | %B |
| | 0.0 | 20 |
| | 0.6 | 20 |
| | 1.00 | 100 |
| | 1.50 | 100 |
| | 1.60 | 20 |
| | 2.5 | 20 |

| | |
|---|---|
| | A: 0.1% formic acid in water |
| | B: 0.1% formic acid in acetonitrile |
| Calibration Standards | Standards were prepared at 2.34, 4.69, 9.38, |
| | 18.8, 37.5, 75.0, 150, 300, 600, 1200, 2400, |
| | and 4800 ng/mL. |
| QC samples | QC samples were prepared at 14.0, 225 and |
| | 3600 ng/mL, analyzed in triplicate. |

Sample concentrations were determined using the best fitting model, either linear or quadratic calibration functions, weighted 1/x, generated by the regression of analyte to IS peak area ratio in the standard samples to their respective concentrations. Acceptance criteria for the analytical runs required that the back-calculated values of the standards and the QC samples fell within ±20% of their nominal values, except for the lowest standard or lower limit of quantitation (LLOQ) for which the acceptance criterion was ±25%. At least six out of twelve standard points had to show back-calculated values within ±20% of their nominal concentrations for the calibration to be accepted. At least three QC samples, one at each concentration, had to show back-calculated values within ±20% of their nominal concentrations for the whole sample batch to be valid.

Data Processing and Analysis: All statistics were calculated using GraphPad Prism version 8 software. Concentration response curves were generated using the Hill Langmuir equation:

Y=B+(T−B)×xⁿ/(IC₅₀ⁿ+xⁿ), Equation 1

where:
B=bottom, set as 0.
T=top, set as 1.
n=the Hill coefficient, constrained to less than zero.
IC₅₀=concentration of a compound required for 50% inhibition in vitro.

All group data is expressed as mean. Between-group differences were analyzed using Kruskal-Wallis followed by Dunn's multiple comparison test. Statistical significance was reached at values of p<0.05.

Bioanalysis: All system suitability test (SST), QC, matrix, and solvent blanks met acceptance criteria described in SOP MTD-066. The LLOQ for Compound A and Lacosamide was 2.34 ng/mL for Study 2C, 4.69 ng/mL for both compounds in Studies 2D and 2A, and 2.34 ng/mL and 4.69 ng/mL for Lacosamide and Compound A, respectively, in Study 2B. The upper limit of quantitation (ULOQ) was 4800 ng/mL for both compounds in all studies.

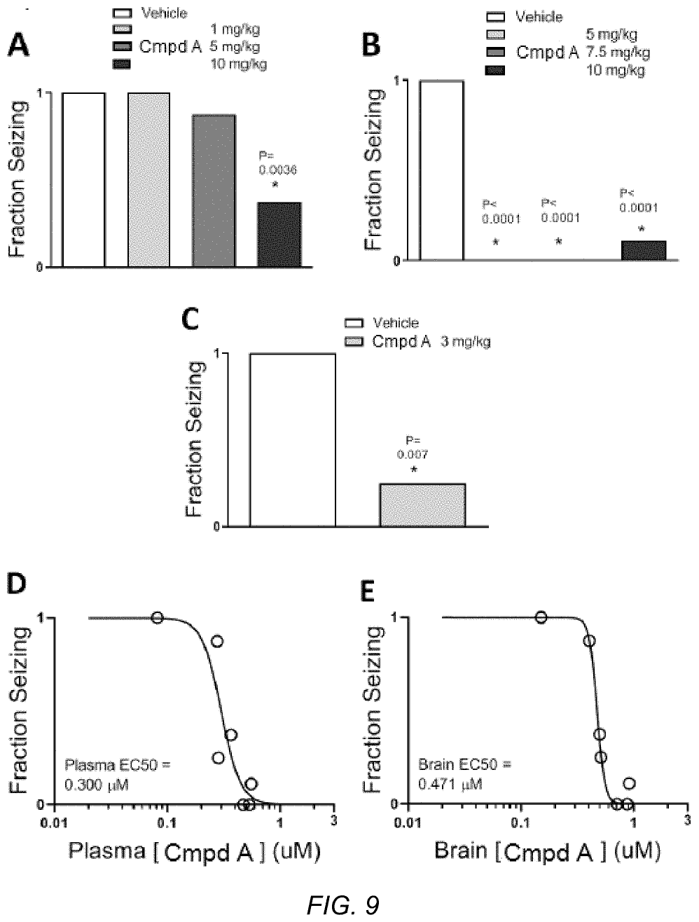

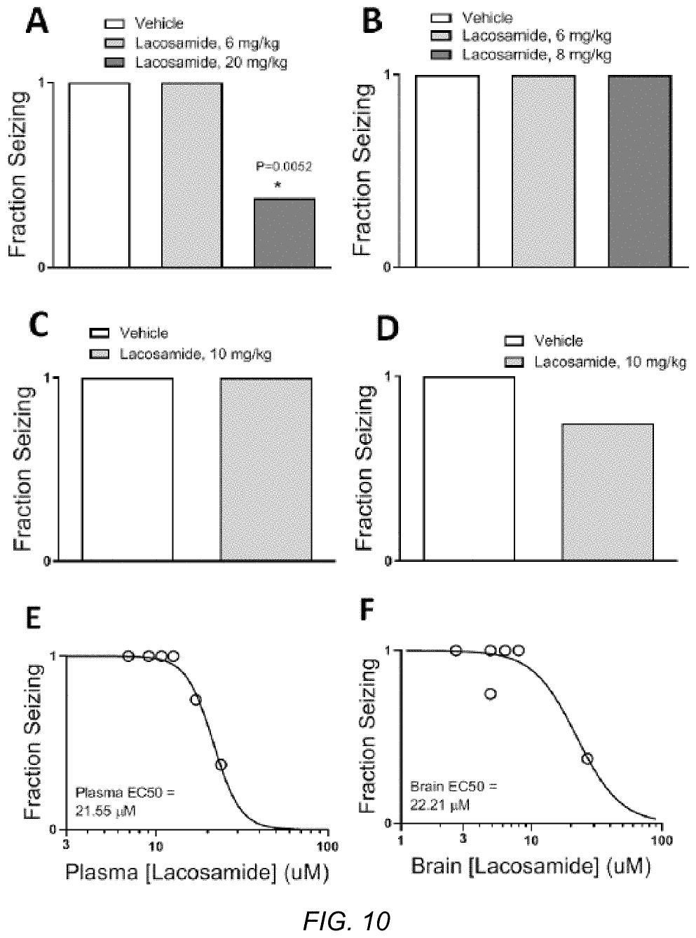

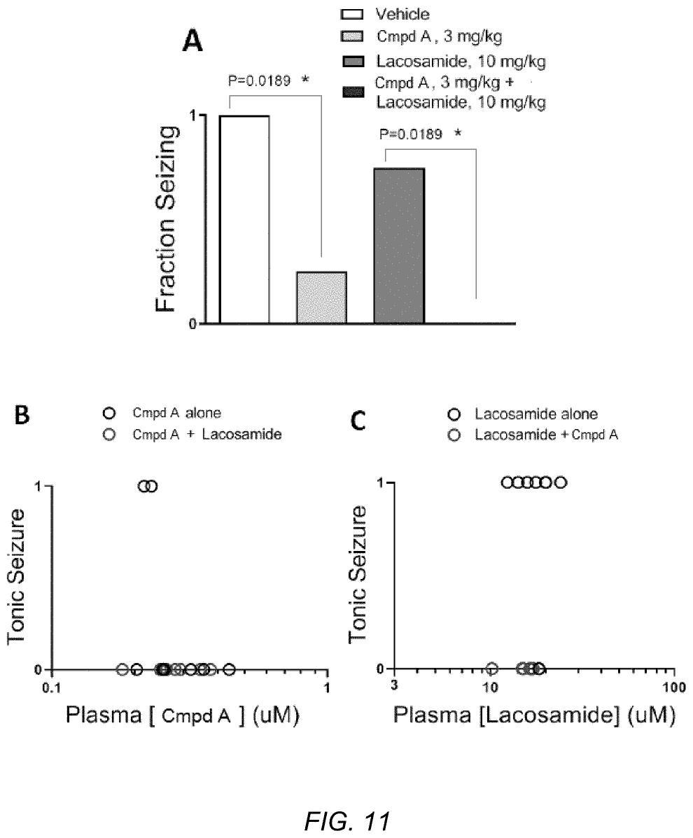

Efficacy in the AC-MES Assay: The results of the AC-MES-induced seizure study in mice are presented in Table 10, Table 11, Table 12, and Table 13, and in FIG. 9, FIG. 10, and FIG. 11.

Dose and Concentration Response of Compound A: The dose and concentration responses of Compound A are shown in FIG. 9. In all three efficacy studies (2A, 2B, and 2C), 100% of vehicle-treated CF-1 mice showed a tonic seizure with extension of hindlimbs. In Study 2A, the fraction of animals dosed with Compound A that showed a tonic seizure response to the AC-MES stimulus decreased from 8/8 at 1 mg/kg, to 7/8 at 5 mg/kg, to 3/8 at 10 mg/kg, suggesting a dose-dependent increase in efficacy. In Study 2B, 5 mg/kg and 10 mg/kg doses showed greater efficacy, with 0/8 and 1/9 animals seizing, respectively, than in Study 2A, which may have resulted from higher plasma concentrations achieved in Study 2B (mean plasma concentrations: 0.470 μM at 5 mg/kg and 0.553 μM at 10 mg/kg) than in Study 2A (mean plasma concentrations: 0.276 μM at 5 mg/kg and 0.366 μM at 10 mg/kg). In addition, Studies 2A and 2B were performed a week apart from each other, which also may explain the difference in efficacy. In Study 2C, a 3 mg/kg dose of Compound A showed 2/8 animals seizing at a mean plasma concentration of 0.284 μM. In all three studies (2A, 2B, and 2C), the difference between Compound A-treated groups and vehicle-dosed groups reached statistical significance (p-values are shown in FIG. 9).

One mouse from the 10 mg/kg group in Study 2A and three mice in study 2B (two mice from the 10 mg/kg group and one mouse from the 7.5 mg/kg group) showed behavioral signs (tremors, decreased activity, and splayed hindlimbs) (plasma concentrations: 2A: 0.391 μM; 2B: 10 mg/kg: 0.608 and 0.516 μM, 7.5 mg/kg: 0.746 μM). The concentrations of Compound A reached in brain tissue and plasma were dose-linear (FIG. 9).

The composite concentration response curve of Compound A from the three studies showed an EC₅₀ for plasma of 0.300 μM and an EC₅₀ for brain tissue of 0.471 μM (FIG. 9).

5.2.2 Results

Prior to testing of Compound A in combination with Lacosamide in the AC-MES model, Lacosamide was tested at different doses in a separate study (2D) and to separate groups of mice within the studies summarized for Compound A above, to establish a full dose-response. The composite dose- and concentration-response of Lacosamide from these 4 studies are shown in FIG. 10. Lacosamide showed a dose- and concentration-dependent effect against AC-MES-induced tonic seizures with maximal effect at 20 mg/kg with 3/8 animals seizing (mean plasma concentration of 23.9 μM). The fraction of animals seizing in the 20 mg/kg Lacosamide-treated group was significantly different from the vehicle-treated group (p-value=0.0052). The concentration-response curve analysis of Lacosamide showed an EC₅₀ for plasma of 21.6 μM and an EC₅₀ for brain tissue of 22.2 μM. For the combination study of Compound A+Lacosamide, Study 2C, a dose of 10 mg/kg Lacosamide and 3 mg/kg of Compound A was chosen, both of which showed minimal efficacy when dosed alone in the AC-MES model.

Combining Compound A (3 mg/kg administered PO 0.5 hours before AC-MES) with Lacosamide (10 mg/kg administered PO 2 hours before AC-MES) abolished tonic seizures (Table 12). The difference between the Lacosamide-treated group (Lacosamide alone at 10 mg/kg with 6/8 animals seizing) and the combination group (Compound A+Lacosamide with 0/8 animals seizing) was statistically significant (p value=0.0189), whereas the difference between the Compound A-treated group (Compound A alone at 3 mg/kg with 2/8 animals seizing) and the combination group was not statistically significant. Plasma concentrations of Compound A (administered at 3 mg/kg) and Lacosamide (administered at 10 mg/kg) were similar whether dosed alone or in combination, suggesting that a greater efficacy observed in the combination group was not a result of higher exposure of either compound. The mean plasma concentrations achieved were: 0.284 μM for Compound A when administered alone at 3 mg/kg; 0.279 μM for Compound A when administered at 3 mg/kg in combination with Lacosamide; 17.1 μM for Lacosamide when administered alone at 10 mg/kg; and 15.5 μM for Lacosamide when administered at 10 mg/kg in combination with Compound A.

TABLE 10
Study 2A: Summary of the Anticonvulsant Effects of Compound A
and Lacosamide in the Mouse AC-MES Following a Single Oral Dose

| | Mean | Mean | | | Fraction with |
|---|---|---|---|---|---|
| | Plasma | Brain | B/P | Fraction | Behavioral |
| Compound ID and Dose | (μM) | (μM) | Ratio | Seizing | Signs |
| Vehicle | n/a | n/a | n/a | 8/8 | 0/8 |
| Compound A (1 mg/kg) | 0.08 | 0.15 | 1.85 | 8/8 | 0/8 |
| Compound A (5 mg/kg) | 0.28 | 0.40 | 1.45 | 7/8 | 0/8 |
| Compound A (10 mg/kg) | 0.37 | 0.50 | 1.36 | 3/8 | 1/8 |
| Lacosamide (6 mg/kg) | 9.07 | 4.85 | 0.54 | 8/8 | 0/8 |
| Lacosamide (8 mg/kg) | 10.8 | 6.27 | 0.58 | 8/8 | 0/8 |

B/P: brain to plasma ratio; n/a: not applicable.

TABLE 11
Study 2B: Summary of the Anticonvulsant Effects of Compound A
and Lacosamide in the Mouse AC-MES Following a Single Oral Dose

| | Mean | Mean | | | Fraction with |
|---|---|---|---|---|---|
| | Plasma | Brain | B/P | Fraction | Behavioral |
| Compound ID and Dose | (μM) | (μM) | Ratio | Seizing | Signs |
| Vehicle | n/a | n/a | n/a | 8/8 | 0/8 |
| Compound A (5 mg/kg) | 1 | 0.71 | 1.51 | 0/8 | 0/8 |
| Compound A (7.5 mg/kg) | 0.53 | 0.87 | 1.64 | 0/7 | 1/7 |
| Compound A (10 mg/kg) | 0.55 | 0.91 | 1.64 | 1/9 | 2/9 |
| Lacosamide (10 mg/kg) | 12.6 | 2.63 | 0.21 | 8/8 | 0/8 |

B/P: brain to plasma ratio; n/a: not applicable.

TABLE 12
Study 2C: Summary of the Anticonvulsant Effects of Compound A
and Lacosamide in Combination in the Mouse AC-MES Following
a Single Oral Dose

| | | | | | Fraction |
|---|---|---|---|---|---|
| | | | | | with |
| | Mean | Mean | | | Behav- |
| | Plasma | Brain | B/P | Fraction | ioral |
| Compound ID and Dose | (μM) | (μM) | Ratio | Seizing | Signs |
| Vehicle | n/a | n/a | n/a | 8/8 | 0/8 |
| Compound A (3 mg/kg) | 0.28 | 0.51 | 1.79 | 2/8 | 0/8 |
| Lacosamide (10 mg/kg) | 17.1 | 4.84 | 0.28 | 6/8 | 0/8 |
| Compound A (3 mg/kg) + | 0.28 + | 0.54 + | 1.95 + | 0/8 | 0/8 |
| Lacosamide (10 mg/kg) | 15.5 | 4.52 | 0.29 | | |

B/P: brain to plasma ratio; n/a: not applicable.

TABLE 13
Study 2D: Summary of the Anticonvulsant Effects of Lacosamide
in the Mouse AC-MES Following a Single Oral Dose

| | Mean | Mean | | | Fraction with |
|---|---|---|---|---|---|
| Compound ID | Plasma | Brain | B/P | Fraction | Behavioral |
| and Dose | (μM) | (μM) | Ratio | Seizing | Signs |
| Vehicle | n/a | n/a | n/a | 8/8 | 0/8 |
| Lacosamide (6 mg/kg) | 6.90 | 7.95 | 1.15 | 8/8 | 0/8 |
| Lacosamide (20 mg/kg) | 23.9 | 26.8 | 1.12 | 3/8 | 0/8 |

B/P: brain to plasma ratio; n/a: not applicable.

5.2.1 Conclusions

Compound A and Lacosamide demonstrated concentration-dependent efficacy in the CF-1 mouse AC-MES assay. Plasma and brain Compound A EC₅₀ values projected from the concentration-response curve analysis were 0.30 μM and 0.47 μM, respectively. The combination of Compound A at 3 mg/kg and Lacosamide at 10 mg/kg led to complete suppression of tonic seizures compared to partial suppression when Compound A or Lacosamide was administered alone in the mouse AC-MES assay.

Five oral (PO) doses of Compound A were each tested in the AC-MES model. Thirty minutes after administration of Compound A at 1 mg/kg (n=8), 3 mg/kg (n=8), 5 mg/kg (n=16), 7.5 mg/kg (n=7), and 10 mg/kg (n=17) via oral gavage to male CF-1 mice (body weights 28.2 to 43.8 g), the mice underwent a 60 Hz corneal electrical stimulus (0.2 seconds duration, 40 mA). This stimulus elicited a tonic hindlimb extension in all vehicle-dosed animals. Any animal that did not show hindlimb extension in response to the electrical stimulus was considered protected. An observational neurological assessment (qualitative test) was also conducted at the time of testing as a tolerability screen. Terminal plasma and brain samples were collected from all animals to obtain the levels of Compound A concentration, and to understand the relationship between efficacy and drug concentration.

After a single oral dose, Compound A showed concentration-dependent effect against AC-MES-induced tonic seizures with maximal effect of 0/8 animals seizing at a plasma concentration of 0.47 μM (from Study 2B at 5 mg/kg). In Study 2A (at 1 mg/kg, n=8; 5 mg/kg, n=8; and 10 mg/kg, n=8; study date 28 Mar. 2019), the fraction of animals that showed a tonic seizure response to the AC-MES stimulus following a 30 minute pre-treatment with Compound A decreased from 8/8 (mean plasma concentration of 0.0815 μM; 1 mg/kg), to 7/8 (mean plasma concentration of 0.28 μM; 5 mg/kg), and to 3/8 (mean plasma concentration of 0.37 μM; 10 mg/kg). In Study 2B (5 mg/kg, n=8; 7.5 mg/kg, n=7 and 10 mg/kg, n=9; study date 5 Jun. 2019), 5 mg/kg (mean plasma concentration of 0.47 μM) and 10 mg/kg (mean plasma concentration of 0.55 μM) doses of Compound A showed strong efficacy (0/8 and 1/9 animals seizing, respectively). In Study 2C (3 mg/kg, n=8; study date 11 Jun. 2019), 3 mg/kg dose of Compound A showed 2/8 animals seizing at a plasma concentration of 0.28 μM. In all three efficacy studies (2A, 2B, and 2C) the difference between Compound A-treated groups and vehicle-dosed groups reached statistical significance. Data from all 3 studies were combined for concentration-response curve analysis of Compound A when dosed alone. The concentration-response curve of Compound A showed a half-maximum effective concentration (EC₅₀) for plasma of 0.30 μM and an EC₅₀ for brain tissue of 0.47 μM.

One mouse from the 10 mg/kg group in Study 2A and three mice in study 2B (two mice from the 10 mg/kg group and one mouse from the 7.5 mg/kg group) showed behavioral signs (tremors, decreased activity, and splayed hindlimbs) (plasma concentrations: 2A: 0.39 μM; 2B: 10 mg/kg: 0.61 and 0.52 μM, 7.5 mg/kg: 0.75 μM).

Prior to testing of Compound A in combination with Lacosamide in the AC-MES model (Study 2C), Lacosamide was tested alone at different doses to establish a full dose-response. Four PO doses of Lacosamide were each tested in the AC-MES model separately (Study 2D: 6 mg/kg and 20 mg/kg, n=8 per group, study date 14 Mar. 2019; Study 2A: 6 mg/kg and 8 mg/kg, n=8 per group, study date 28 May 2019; Study 2B: 10 mg/kg, n=8, study date 5 Jun. 2019; and Study 2C: 10 mg/kg, n=8, study date 11 Jun. 2019) and as part of the studies. Two hours after administration of Lacosamide at 6 mg/kg (n=16), 8 mg/kg (n=8), 10 mg/kg (n=16), and 20 mg/kg (n=8) via oral gavage to male CF-1 mice (body weights 28.2 to 43.8 g), the mice underwent an AC-MES stimulus. Lacosamide showed dose- and concentration-dependent effect against AC-MES-induced tonic seizures with maximal effect at 20 mg/kg with 3/8 animals seizing (mean plasma concentration of 23.9 μM) and minimal effect at lower doses of 6, 8, and 10 mg/kg. Data from all 4 studies (2A, 2B, 2C, and 2D) were combined for concentration response curve analysis of Lacosamide when dosed alone. The concentration response curve of Lacosamide showed an EC₅₀ for plasma of 21.6 μM and an EC₅₀ for brain tissue of 22.2 μM. Based on dose-response studies, a dose of 10 mg/kg Lacosamide and 3 mg/kg of Compound A was chosen that showed minimal efficacy when dosed alone for the combination study in the AC-MES model.

Combining Compound A (3 mg/kg PO, 0.5 hours before AC-MES) with Lacosamide (10 mg/kg PO, 2 hours before AC-MES) in male CF-1 mice completely suppressed tonic seizures compared to partial suppression when Compound A or Lacosamide were dosed alone (Compound A alone at 3 mg/kg: 2/8 animals seizing; Lacosamide alone at 10 mg/kg: 6/8 animals seizing; Compound A+Lacosamide: 0/8 animals seizing; vehicle: 8/8 animals seizing). Plasma concentrations of Compound A (3 mg/kg) and Lacosamide (10 mg/kg) were similar whether dosed alone or in combination, suggesting that the maximal efficacy observed in the combination group was not a result of higher exposure of either compound. The mean total plasma concentrations achieved were: Compound A when dosed alone at 3 mg/kg: 0.283 μM; Compound A when dosed in combination at 3 mg/kg: 0.279 μM; Lacosamide when dosed alone at 10 mg/kg: 17.1 μM; Lacosamide when dosed in combination at 10 mg/kg: 15.5 μM.

### 5.3. Example 3. Compound A Alone and in Combination with Levetiracetam in 6 Hz Psychomotor Seizure Assay

The objective of this study was to evaluate the interaction of Compound A with Levetiracetam in a mouse model of seizures sensitive to each compound, the 6 Hz psychomotor seizure assay (Barton et al., Epilepsy Res. 2001; 47(3):217-227). It was evaluated whether the combination of both compounds is favorable or unfavorable for efficacy by dosing Compound A and Levetiracetam alone and in combination, each at a dose that produces sub-maximal efficacy on its own. Compound A and Levetiracetam were each administered at a dose that produces sub-maximal efficacy in the 6 Hz assay on its own to allow detection of a change in efficacy in either direction when these compounds were combined. Plasma and brain samples were analyzed to gain an understanding of how the combination of Compound A and Levetiracetam may affect pharmacokinetics and pharmacodynamics.

5.3.1 Materials and Methods

Test Compound—Compound A

| | |
|---|---|
| Identity: | Compound A |
| Batch Number: | 12 |
| Physical Description: | White solid |
| Purity (%): | 98.8 |
| Supplier: | Olon Recerca Biosciences |
| | (Lot No. 60367-18-002) |

Reference Compound—Levetiracetam

| | |
|---|---|
| Identity: | Levetiracetam |
| Batch Number: | 03 |
| Physical Description: | White powder |
| Purity (%): | 98% |
| Supplier: | Toronto Research Chemicals; |
| | Catalog No. L331500, Lot No. 3-JSH-3-1 |

Vehicle: Compound A formulation F2: 5% dimethyl sulfoxide (DMSO), 0.5% methyl cellulose in deionized (DI) water. Levetiracetam formulation F1: 0.5% w:w methyl cellulose, 0.2% v:v Tween 80 in DI water.

Dose Formulation: The appropriate amount of Compound A (no correction for purity) was weighed and dissolved in DMSO at 20× the intended final concentration. The 20×DMSO stock solution of Compound A was diluted 20-fold with 0.5% methyl cellulose in DI water to achieve the final desired concentration. The resulting Compound A suspension was stirred or vortex-mixed to yield a homogenous suspension. The formulation was kept at room temperature and stirred continuously or vortex-mixed prior to each dose administration.

For Levetiracetam, DI water (0.8 L) was heated up to 70° C. to 80° C. Five grams of methyl cellulose was weighed and slowly added in small portions to heated DI water. The mixture was stirred until it formed a homogeneous milky suspension. The suspension was moved to a cold room and stirred overnight to obtain a clear solution. Two mL of Tween 80 were added to the clear solution and diluted up to 1 L with DI water. The vehicle solution was stored at 2° C. to 8° C.

Levetiracetam powder was weighed into vials. An appropriate amount of vehicle was added to the powder then mixed on the IKA-T18 ULTRA TURRAX homogenizer to create a uniform suspension at the desired concentration. The vials were then wrapped in aluminum foil to protect them from light, and placed on a stir plate until the time of dosing.

Test System

| Species/Strain: | CF-1 mice | | |
|---|---|---|---|
| Number and Sex: | 104 males | | |
| Source: | Charles River Laboratories | | |
| Age and Body Weight at | Study ID | Age (days) | Weight Range (g) |
| Initiation of Dosing: | 3A | 60 | 32.7 to 46.0 |
| | 3B | 46 | 29.0 to 37.3 |
| | 3C | 48 | 31.0 to 40.0 |

| | |
|---|---|
| Housing: | Animals were group-housed (4 per cage) in cages that are in |
| | accordance with applicable animal welfare laws and regulations |
| | (CCAC). Temperature was between 18° C. and 25° C. and relative |
| | humidity between 45% and 65%. |
| Acclimation: | Upon arrival the animals were examined to assure their |
| | satisfactory health status and acclimated to the facility for at least |
| | 5 days before being placed on study. |
| Food/Water: | Animals were provided Certified Rodent Chow (Teklad Rodent |
| | Chow #2014) ad libitum. Tap water was available ad libitum. |

Experimental Design: The animals were assigned to treatment groups as indicated in Table 14. All mice were dosed with either vehicle or Compound A via PO gavage (Standard Operating Procedure (SOP) TECH-006) and either vehicle or Levetiracetam by IP injection (TECH-004) at 1 hour prior to seizure induction. The ED₂₀ (4 mg/kg) of Compound A was chosen for combination experiments (based on the dose response experiment, Study 3A). A dose of 300 mg/kg Levetiracetam was chosen based on previous experiments that yielded on average 35% efficacy at this dose.

TABLE 14
Overview of Experimental Groups

| | | | Dose | Formulation | | Route | No. of | Pre- |
|---|---|---|---|---|---|---|---|---|
| | | Dose | Volume | Conc. | | of | Animals | Treatment |
| Study | Cmpd | (mg/kg) | (mL/kg) | (mg/mL) | Vehicle | Admin. | (Sex) | Time (h) |
| 3A | Vehicle | 0 | 10 | n/a | F2 | PO | 8 (male) | 1 |
| | Cpmd A | 1 | 10 | 0.1 | F2 | PO | 8 (male) | 1 |
| | Cpmd A | 3 | 10 | 0.3 | F2 | PO | 8 (male) | 1 |
| | Cpmd A | 5 | 10 | 0.5 | F2 | PO | 8 (male) | 1 |
| | Cpmd A | 8 | 10 | 0.8 | F2 | PO | 8 (male) | 1 |
| 3B | Vehicle | n/a | 10 | n/a | F2 | PO | 8 (male) | 1 |
| | Vehicle | | | | F1 | IP | | |
| | Vehicle | 0 | 10 | n/a | F2 | PO | 8 (male) | 1 |
| | Lev | 300 | | 30 | F1 | IP | | |
| | Cpmd A | 4 | 10 | 0.4 | F2 | PO | 8 (male) | 1 |
| | Vehicle | 0 | | n/a | F1 | IP | | |
| | Cpmd A | 4 | 10 | 0.4 | F2 | PO | 8 (male) | 1 |
| | Lev | 300 | | 30 | F1 | IP | | |
| 3C | Vehicle | n/a | 10 | n/a | F2 | PO | 8 (male) | 1 |
| | Vehicle | | | | F1 | IP | | |
| | Vehicle | 0 | 10 | n/a | F2 | PO | 8 (male) | 1 |
| | Lev | 300 | | 30 | F1 | IP | | |
| | Cpmd A | 4 | 10 | 0.4 | F2 | PO | 8 (male) | 1 |
| | Vehicle | 0 | | n/a | F1 | IP | | |
| | Cpmd A | 4 | 10 | 0.4 | F2 | PO | 8 (male) | 1 |
| | Lev | 300 | | 30 | F1 | IP | | |

Admin: administration; Conc.: concentration; Cpmd.: Compound; IP: intraperitoneal; Lev: Levetiracetam; n/a: not applicable; PO: per os, oral.

Randomization and Blinding: The experimenter administering the compound assigned each animal randomly to a treatment group. A different experimenter, blinded to treatment group allocation, performed the test. All animals tested in a given experiment had an equal chance of assignment to any treatment group.

Observation for Clinical Signs: All animals dosed with compound were observed for abnormal behavior for 10 minutes after dosing by the experimenter who dosed the animals and again at the time of stimulation by the experimenter who tested the animals. Any qualitative changes from normal behavior were recorded.

6 Hz Psychomotor Seizure Assay: All animals were brought into the experiment room at least 1 hour prior to the electrical stimulation Immediately prior to the assay, a drop of Alcaine (proparacaine hydrochloride, 0.5%) was applied to each of the mouse's eyes. The animal was then securely restrained and a pair of electrodes was applied to the eyes (Electro Convulsive Therapy Unit 57800, Ugo Basile). A 3-second stimulus was elicited with a foot pedal at 34 milliampere, 6 Hz, and 0.2 millisecond pulse width Immediately after completion of the stimulus, the animal was placed in a Plexiglas cylinder and seizure behavior (jaw clonus, forelimb clonus, and Straub tail) was recorded (according to TECH-036). An animal was considered protected from seizure if it showed none of the three typical psychomotor seizure behaviors (jaw clonus, forelimb clonus, Straub tail) induced by this stimulation protocol in control animals.

Sample Collection and Preparation: Mice were anesthetized by isoflurane inhalation (TECH-018) until they reached a surgical plane of anesthesia. Then a syringe (1 mL syringe with a 22 gauge needle) was inserted under the sternum into the heart (TECH-031). Approximately 0.5 mL of blood was collected, deposited into a K2EDTA tube, and stored on ice. Animals were then euthanized by cervical dislocation (TECH-018). Brains were removed, placed into a pre-weighed vial, and snap frozen on dry ice. At the end of the sample collection, blood was centrifuged at 4000 rpm for 10 minutes at 4° C., and the plasma pipetted into a labeled tube. All samples were stored in a −80° C. freezer until bioanalysis (8 days after sample collection for Studies 3A and 3B; 20 days after sample collection for Study 3C).

Plasma and Tissue Sample Analysis: Bead mill polypropylene tubes containing weighed brain tissues were thawed at room temperature and 3 mL of homogenization solvent (water:acetonitrile (1:1, v:v)) was added. The tubes were placed in the Bead Mill Homogenizer (Bead Ruptor Elite Model, Omni International) and shaken at a velocity of 3.70 m/s for a single cycle lasting 30 seconds. The homogenized tube was centrifuged at 4000 rpm for 20 minutes, the supernatant was transferred to a 1.5 mL Eppendorf tube, and stored frozen at −80° C. until analysis. All samples including plasma and brain homogenate extracts (including calibration and quality control (QC) samples prepared in K₂EDTA rat plasma), were extracted by protein precipitation. To each 50 μL aliquot of sample, 50 μL of internal standard solution (2500 ng/mL of (S)-5-chloro-4-((1-(5-chloro-2-fluorophenyl)ethyl)amino)-2-fluoro-N-(pyrazin-2-yl)benzenesulfonamide and 44(24(7-azabicyclo[2.2.1]heptan-7-yl)methyl)-6-fluorobenzyl)amino)-2,6-difluoro-N-(isothiazol-3-yl)benzenesulfonamide for Compound A and Levetiracetam, respectively, in water/acetonitrile (1:1)), 50 μL of 6% (v/v) phosphoric acid in water, and then 200 μL to acetonitrile was added. The samples in the 1.5 mL tubes were vortex mixed and then centrifuged for 20 minutes at 13,000 rpm. Fifty microliters of the supernatant was mixed with 150 μL of water/acetonitrile (1:1) in a 96 well plate which was centrifuged post mixing for 20 minutes at 4000 rpm, and was then ready for analysis by liquid chromatography tandem mass spectrometry (LC-MS/MS).

Samples were analyzed by a research-grade LC-MS/MS method as follows:

| Instrument: | Sciex TQ-5500 | |
|---|---|---|
| Matrix: | Mouse plasma (K₂EDTA) | |
| | Mouse brain homogenate | |
| Analyte: | Compound A | |
| Internal Standard (IS): | (S)-5-chloro-4-((1-(5-chloro-2-fluorophenyl)ethyl)amino)-2-fluoro-N- | |
| | (pyrazin-2-yl)benzenesulfonamide | |
| MS Conditions: | ESI (electrospray ionization): positive | |
| | Compound A: [M+H]⁺ m/z 369.2/247.2 | |
| | (S)-5-chloro-4-((1-(5-chloro-2-fluorophenyl)ethyl)amino)-2-fluoro-N- | |
| | (pyrazin-2-yl)benzenesulfonamide (IS): [M+H]⁺ m/z 458.793/157 | |
| Ultra-high performance | Time (min) | %B |
| liquid chromatography | 0.0 | 10 |
| (UHPLC) Conditions: | 0.2 | 10 |
| | 3.4 | 86 |
| | 3.5 | 100 |
| | 4.0 | 100 |
| | 4.1 | 10 |
| | 5.0 | 10 |
| Calibration Standards | A: 0.1% formic acid in water | |
| | B: 0.1% formic acid in acetonitrile | |
| | Standards were prepared at 2.34, 4.69, 9.38, 18.8, 37.5, 75, 150, 300, | |
| | 600, 1200, 2400, and 4800 ng/mL for Studies 3A and 3B. | |
| | Standards were prepared at 0.586, 1.17, 2.34, 4.69, 9.38, 18.8, 36.5, | |
| | 75.0, 150, 300, 600, and 1200 ng/mL for Study 3C. | |
| Analyte: | Levetiracetam | |
| Internal Standard (IS): | 4-((2-((7-azabicyclo[2.2.1]heptan-7-yl)methyl)-6-fluorobenzyl)amino)- | |
| | 2,6-difluoro-N-(isothiazol-3-yl)benzenesulfonamide | |
| MS Conditions: | ESI: positive | |
| | Levetiracetam: [M+H]⁺ m/z 171/126 | |
| | 4-((2-((7-azabicyclo[2.2.1]heptan-7-yl)methyl)-6-fluorobenzyl)amino)- | |
| | 2,6-difluoro-A-(isothiazol-3-yl)benzenesulfonamide (IS): [M+H]⁺ m/z | |
| | 509/218 | |
| UHPLC Conditions: | Time (min) | %B |
| | 0.0 | 20 |
| | 0.6 | 20 |
| | 1.0 | 100 |
| | 1.7 | 100 |
| | 1.8 | 20 |
| | 3.0 | 20 |

| | |
|---|---|
| Calibration Standards | A: 0.1% formic acid in water |
| | B: 0.1% formic acid in acetonitrile |
| | Standards were prepared at 4.69, 9.38, 18.8, 37.5, 75, 150, 300, 600, |
| | 1200, 2400, 4800, and 9600 ng/mL. |

Data Processing and Analysis: Statistical data analysis was performed using GraphPad Prism (version 8.2.1). Dose-response data was analyzed using a Kruskal-Wallis test, followed by Dunn's multiple comparisons. A p-value <0.05 was considered significant. Dose- and concentration-response curves were generated using the Hill Langmuir equation:

Y=B+(T−B)×xⁿ/(IC₅₀ⁿ+xⁿ), Equation 1

where:
B=bottom, set as 0.
T=top, set as 1.
n=the Hill coefficient, constrained to less than zero.
IC₅₀=concentration of a compound required for 50% inhibition in vitro.

All data is reported to three significant figures and all group data is reported as mean±SD unless stated otherwise.

5.3.2 Results

Bioanalysis: All system suitability tests, QC samples, matrix, and solvent blanks met acceptance criteria. Analysis parameters are tabulated in Table 15. QC samples at each concentration were analyzed in triplicate.

TABLE 15
Bioanalysis Parameters

| | Cmpd A | Cmpd A | Cmpd A | Lev | Lev | | Samples |
|---|---|---|---|---|---|---|---|
| | LLOQ | ULOQ | QC | LLOQ | ULOQ | Lev QC | Below |
| Study | (ng/mL) | (ng/mL) | (ng/mL) | (ng/mL) | (ng/mL) | (ng/mL) | LLOQ |
| 3A | 2.34 | 4800 | 14 | n/a | n/a | n/a | none |
| | | | 225 | | | | |
| | | | 3600 | | | | |
| 3B | 2.34 | 4800 | 14 | 4.69 | 9600 | 28 | 2 plasma |
| | | | 225 | | | 450 | 7 brain |
| | | | 3600 | | | 7200 | |
| 3C | 1.17 | 1200 | 3.52 | 9.38 | 9600 | 28 | 1 plasma |
| | | | 56.25 | | | 450 | 1 brain |
| | | | 900 | | | 7200 | |

Lev: Levetiracetam; LLOQ: lower limit of quantitation; n/a: not applicable; ULOQ: upper limit of quantitation.

Clinical Observations: One animal administered 8 mg/kg Compound A (plasma: 1.16 μM; brain: 1.65 μM) showed tremor and was cold to the touch at the time of testing.

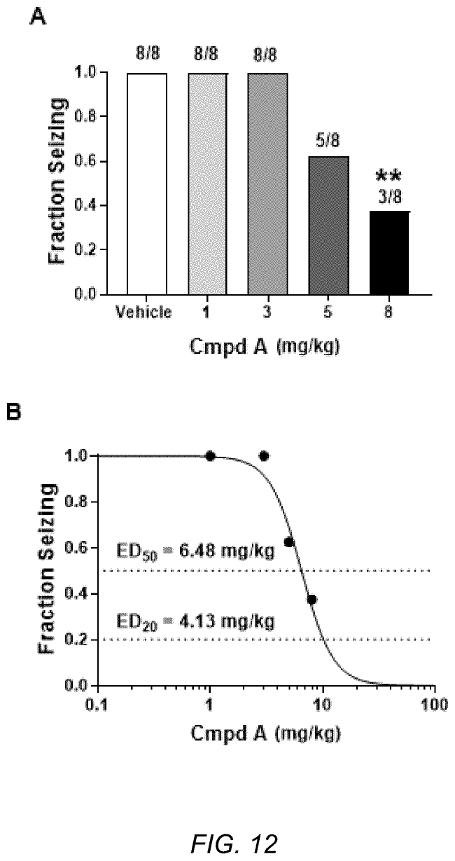

Dose Response of Compound A in the 6 Hz Psychomotor Seizure Assay (Study 3A): Compound A dosed PO 1 hour prior to corneal stimulation showed dose-dependent efficacy with 8/8 animals seizing at 1 mg/kg and 3 mg/kg, 5/8 animals seizing at 5 mg/kg, and 3/8 animals seizing at 8 mg/kg. Seizure protection was significantly different from vehicle at 8 mg/kg Compound A (p=0.0081; FIG. 12A). The dose response curve projects an ED₅₀ of 6.48 mg/kg and an ED₂₀ of 4.13 mg/kg at a Hill coefficient of n=−3.09 (FIG. 12B).

The individual animal plasma and brain pharmacokinetic-pharmacodynamic relationships are shown in FIG. 12A and FIG. 12B, respectively. Results and exposure by group are tabulated in Table 16. Efficacy of Compound A was concentration-dependent with a plasma EC₅₀ of 0.35 μM and a brain EC₅₀ of 0.54 μM at a Hill coefficient of n=−1.95 and n=−2.17, respectively (FIG. 12C and FIG. 12D).

TABLE 16
Efficacy and Exposure of Compound A Following a Single PO
Administration of Different Doses to CF-1 Mice (Study 3A)

| | Dose | Plasma | Plasma SD | Brain | Brain SD | Fraction |
|---|---|---|---|---|---|---|
| Compound | (mg/kg) | (μM) | (μM) | (μM) | (μM) | Seizing |
| Vehicle | n/a | n/a | n/a | n/a | n/a | 8/8 |
| Cmpd A | 1 | 0.04 | 0.01 | 0.09 | 0.06 | 8/8 |
| Cmpd A | 3 | 0.15 | 0.06 | 0.26 | 0.10 | 8/8 |
| Cmpd A | 5 | 0.18 | 0.07 | 0.29 | 0.16 | 5/8 |
| Cmpd A | 8 | 0.47 | 0.29 | 0.70 | 0.41 | 3/8 |

n/a: not applicable.

Combination of Compound A and Levetiracetam in the 6 Hz Psychomotor Seizure Assay (Studies 3B and 3C): To assess whether the combination of Compound A and Levetiracetam is favorable or unfavorable for efficacy in the 6 Hz psychomotor seizure assay, a sub-maximal dose was selected for each compound. The ED₂₀ of Compound A was determined to be 4 mg/kg in the dose-response experiment above. In previous studies, on average 35% efficacy was achieved at 300 mg/kg Levetiracetam, with considerable variability among the studies. In two identically designed experiments (Studies 3B and 3C, see above and Table 14), the combination of 4 mg/kg Compound A and 300 mg/kg Levetiracetam was significantly more efficacious in the 6 Hz psychomotor seizure assay than either compound alone (Table 16).

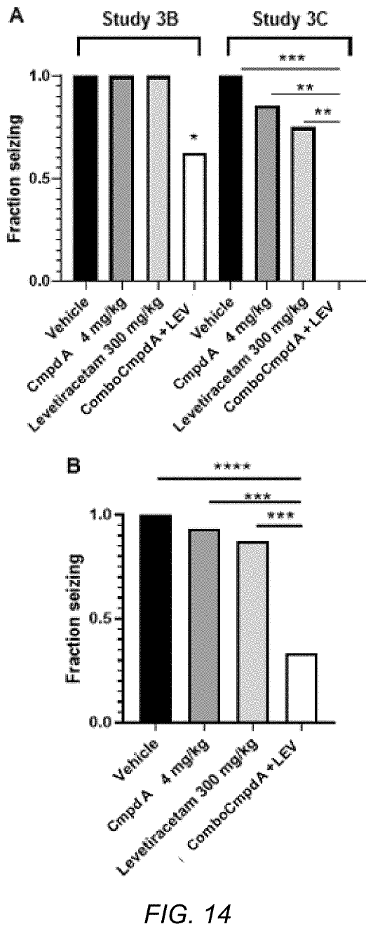

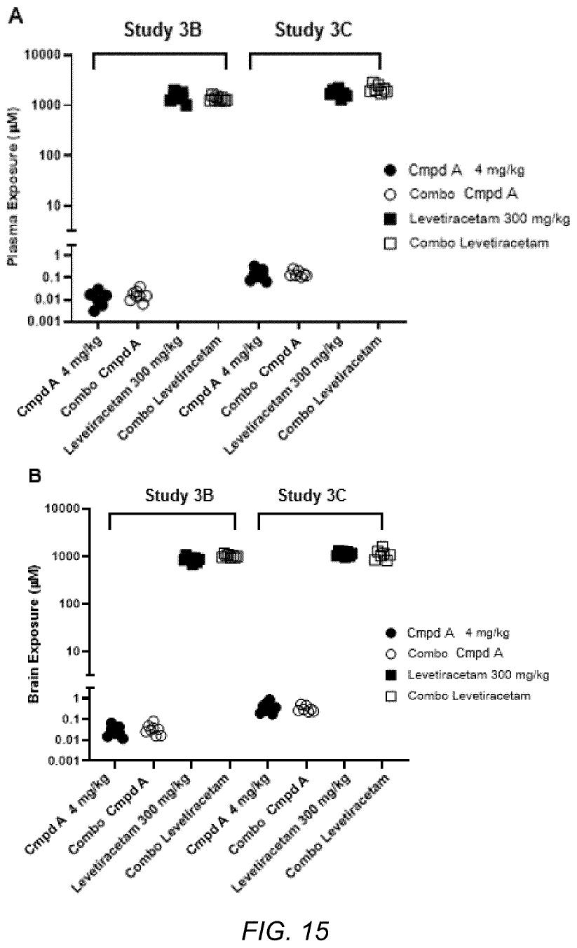

Study 3B: In Study 3B, Compound A or Levetiracetam dosed alone did not result in protection from seizure (plasma and brain concentrations of Compound A were much lower than expected), but combining both compounds protected 3/8 animals from seizing (FIG. 14A, Table 12). The benefit of combination-dosing was significant compared to vehicle and compared to either compound dosed alone (p=0.034). The concentrations of Compound A and Levetiracetam reached in plasma (Compound A: 0.014±0.009 μM; Levetiracetam: 1500±320 μM) and brain (Compound A: 0.03±0.02 μM; Levetiracetam: 861±120 μM) were comparable between the single dose and combination-dose groups (FIG. 14, Table 17). Thus, the beneficial effect of the combination of Compound A and Levetiracetam cannot be explained by increased exposure of either compound (FIG. 15).

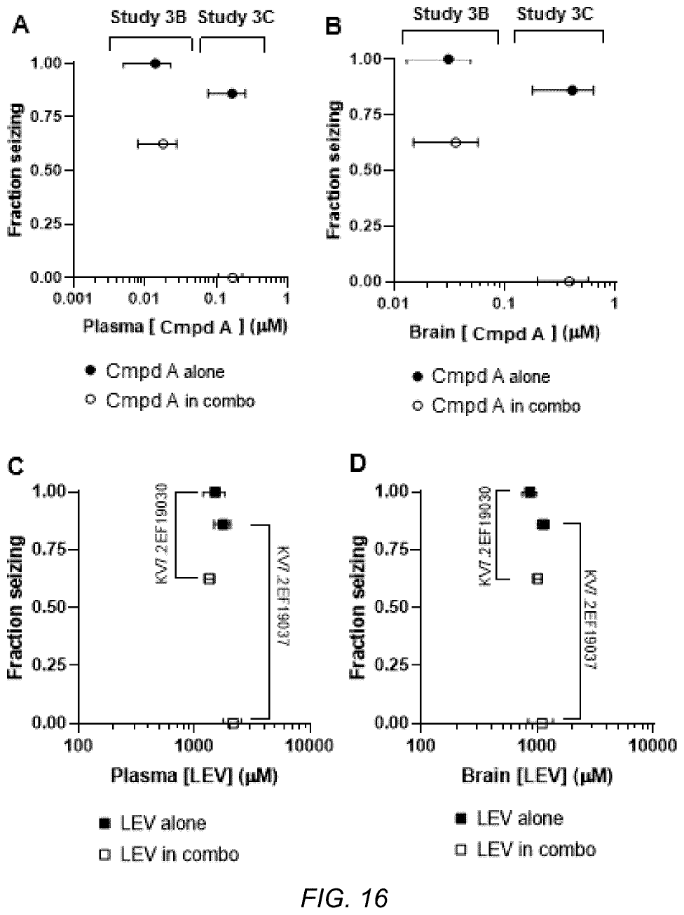

Study 3C: In Study 3C, the design of Study 3B was repeated. This time, the same dose of 4 mg/kg Compound A resulted in more than 10-fold higher concentrations of Compound A in plasma (0.17±0.09 μM vs. 0.01±0.01 μM) and brain (0.41±0.23 μM vs. 0.03±0.02 μM) than in Study 3B (FIG. 15, Table 17). Concentrations of Levetiracetam were also slightly higher than in Study 3B (brain: 1130±130 μM vs. 861±120 μM, plasma: 1770±287 μM vs. 1500±320 μM; FIG. 15, Table 17). The increased exposures translated into increased efficacy of either compound dosed alone compared to Study 3B: Compound A at 4 mg/kg protected 1/7 animals and Levetiracetam at 300 mg/kg protected 2/8 animals from seizing (FIG. 14A, Table 17). The combination of Compound A and Levetiracetam protected all animals tested in this study (FIG. 14A Table 17; n=7; one animal was excluded because it had no measurable levels of Levetiracetam in plasma or brain). With full efficacy reached following combination dosing of Compound A and Levetiracetam, the effect on efficacy was significantly different not only from vehicle (p=0.0002), but also from either compound dosed alone (p<0.01) Similar to Study 3B, the concentrations of Compound A and Levetiracetam reached in plasma and brain were comparable between the single dose and combination dose groups (FIG. 15, Table 17). Thus, the beneficial effect of the combination of Compound A and Levetiracetam cannot be explained by increased exposure of either compound (FIG. 16).

Studies 3B and 3C Combined: When the dose-response of both experiments is combined, maximal efficacy with either Compound A or Levetiracetam dosed alone was reached with Levetiracetam at 14/16 seizing, while the combination of both compounds resulted in 5/15 animals seizing. The combination of Compound A and Levetiracetam thus protected 66.7% of animals from seizure, which was significantly different from vehicle (p<0.0001), and from either compound alone (p<0.001, FIG. 14B, Table 17).

TABLE 17
Efficacy and Exposure of Compound A and Levetiracetam at 1 Hour Post Dosing Alone or in Combination to CD-1 Mice

| | | | Compound A | | | | Levetiracetam | | | | |
|---|---|---|---|---|---|---|---|---|---|---|---|
| | | | | Plasma | | Brain | | Plasma | | Brain | |
| | | Dose | Plasma | SD | Brain | SD | Plasma | SD | Brain | SD | Fraction |
| Study | Cmpd | (mg/kg) | (μM) | (μM) | (μM) | (μM) | (μM) | (μM) | (μM) | (μM) | Seizing |
| 3B | Veh. | n/a | n/a | n/a | n/a | n/a | n/a | n/a | n/a | n/a | 8/8 |
| | Cmpd A | 4 | 0.01 | 0.01 | 0.03 | 0.02 | n/a | n/a | n/a | n/a | 8/8 |
| | Lev | 300 | n/a | n/a | n/a | n/a | 1500 | 320 | 861 | 120 | 8/8 |
| | Cmpd A + | 4 + 300 | 0.02 | 0.01 | 0.04 | 0.02 | 1350 | 128 | 1010 | 78.8 | 5/8 |
| | Lev | | | | | | | | | | |
| 3C | Veh. | n/a | n/a | n/a | n/a | n/a | n/a | n/a | n/a | n/a | 8/8 |
| | Cmpd A | 4 | 0.17 | 0.09 | 0.41 | 0.23 | n/a | n/a | n/a | n/a | 5/6* |
| | Lev | 300 | n/a | n/a | n/a | n/a | 1770 | 287 | 1130 | 130 | 6/8 |
| | Cmpd A + | 4 + 300 | 0.17 | 0.06 | 0.383 | 0.19 | 2160 | 383 | 1100 | 265 | 0/7** |
| | Lev | | | | | | | | | | |
| Combined | Cmpd A | 4 | 0.10 | 0.10 | 0.22 | 0.25 | n/a | n/a | n/a | n/a | 13/14* |
| | Lev | 300 | n/a | n/a | n/a | n/a | 1640 | 324 | 996 | 185 | 14/16 |
| | Cmpd A + | 4 + 300 | 0.09 | 0.09 | 0.21 | 0.22 | 1730 | 497 | 1050 | 188 | 5/15** |
| | Lev | | | | | | | | | | |

Lev: Levetiracetam; n/a: not applicable.
*Two animals were excluded from efficacy assessment due to a stimulation error. A violation (loss of contact) occurred during the stimulation.
**One animal was excluded because it had no measurable concentration of Levetiracetam in either plasma or brain.

5.3.3 Conclusions

Compound A demonstrated dose-dependent efficacy in the mouse 6 Hz psychomotor assay with an ED₅₀ of 6.48 mg/kg. Efficacy correlated well with plasma and brain concentration of Compound A with a plasma EC₅₀ of 0.35 μM and a brain EC₅₀ of 0.54 μM.

Combination of Compound A at 4 mg/kg and Levetiracetam at 300 mg/kg led to significantly higher efficacy in this assay than was observed with either compound alone in two separate, identically designed experiments (Study 3B: p=0.034; Study 3C: p<0.01). Combination dosing did not significantly affect the plasma or brain exposure of either compound. At comparable brain concentrations of Levetiracetam (1130±130 μM vs. 1100±265 μM; Study 3C), the addition of Compound A to Levetiracetam increased efficacy from 25% (Levetiracetam alone) to 100% (Compound A+Levetiracetam).

First, a dose-response study (3A) was performed for Compound A in the 6 Hz psychomotor assay to determine the dose at which 20% of animals were protected from seizures (ED₂₀). Compound A was administered to male CF-1 mice (body weights: 32.7 to 46.0 g) at 1, 3, 5, and 8 mg/kg by oral gavage to 8 animals per group 1 hour prior to induction of psychomotor seizures via a 3-second corneal stimulation at 34 milliampere (6 Hz; 0.2 milliseconds pulse width). Efficacy in this assay was quantified by calculating the fraction of animals in each group of 8 that was protected from seizure behavior (jaw clonus, forelimb clonus, or Straub tail) following 6 Hz stimulation. Plasma and brain samples were analyzed to assess the concentration-response relationship.

While 1 mg/kg and 3 mg/kg of Compound A did not result in protection from seizure, the fraction of animals seizing was reduced to 5/8 in animals dosed with 5 mg/kg and to 3/8 in the 8 mg/kg group. At 8 mg/kg (plasma: 0.47±0.29 μM; brain: 0.70±0.41 μM; mean±SD), Compound A showed significant protection (p=0.0081) compared to the vehicle group (8/8 seizing). The dose and concentration response curves for Compound A project a dose at which 50% of animals were protected from seizures (ED₅₀) of 6.48 mg/kg, a plasma concentration at which 50% of animals are protected from seizures (EC₅₀) of 0.35 μM, and a brain EC₅₀ of 0.54 μM. The ED₂₀ for Compound A in this assay was calculated to be 4.13 mg/kg.

One animal dosed with 8 mg/kg Compound A showed tremor and was cold to the touch. At 1.16 μM plasma and 1.65 μM brain concentration, this animal had the highest exposure of Compound A among all animals in this experiment.

The combination of Compound A and Levetiracetam at doses that produce sub-maximal efficacy was evaluated in two separate, identically designed experiments (Studies 3B and 3C). Previous experiments had established that an intraperitoneal (IP) injection of 300 mg/kg Levetiracetam at 1 hour prior to stimulation produces 35% efficacy at a 34 milliampere 6 Hz stimulation. The dose-response experiment with Compound A (Study 3A) projected an ED₂₀ of 4 mg/kg when Compound A was dosed orally (PO) at 1 hour prior to stimulation. The following experimental groups (n=8) were then evaluated in each of the two identically designed experiments: 1. Vehicle control (PO and IP); 2. Compound A at 4 mg/kg PO and vehicle IP; 3. Levetiracetam at 300 mg/kg IP and vehicle PO; 4. Compound A at 4 mg/kg PO and Levetiracetam at 300 mg/kg IP. A 3-second corneal stimulation at 34 milliampere (6 Hz, 0.2 millisecond pulse width) was administered at 1 hour following compound administration. Efficacy was evaluated based on the fraction of animals protected from seizure behavior (jaw clonus, forelimb clonus, or Straub tail).

In Study 3B, either compound dosed alone to male CF-1 mice (body weights: 29.0 to 37.3 g) did not result in protection from seizure, but the combination of both compounds protected 3/8 animals from seizing (p=0.034). The concentrations of Compound A and Levetiracetam reached in plasma (Compound A: 0.014±0.009 μM; Levetiracetam: 1500±320 μM) and brain (Compound A: 0.03±0.02 μM; Levetiracetam: 861±120 μM) were comparable between the single dose and combination dose groups, but overall were much lower than expected for a 4 mg/kg dose.

In Study 3C, conducted in male CF-1 mice (body weights: 31.0 to 40.0 g), 4 mg/kg Compound A resulted in more than 10-fold higher concentrations of Compound A in plasma (0.167±0.0897 μM vs. 0.014±0.009 μM) and brain (0.41±0.23 μM vs. 0.03±0.02 μM) than in Study 3B. Concentrations of Levetiracetam were also slightly higher than in 3B (brain: 1130±130 μM vs. 861±120 μM; plasma: 1770±287 μM vs. 1500±320 04). The increased exposures translated into increased efficacy of either compound dosed alone: Compound A at 4 mg/kg protected 1/7 animals, and Levetiracetam at 300 mg/kg protected 2/8 animals from seizing. The combination of Compound A and Levetiracetam protected all animals tested in this group (7/7 protected; p<0.01) compared to either compound alone. At comparable brain concentrations of Levetiracetam (1130±130 μM vs. 1100±265 μM, Study 3C), the addition of Compound A to Levetiracetam increased efficacy from 25% (Levetiracetam alone) to 100% (Compound A+Levetiracetam).

### 5.4. Example 4. Anticonvulsant Effect of Compound A Alone and in Combination with Cenobamate in the AC-MES Assay in CF-1 Mice

The efficacy of Compound A and its pharmacological interaction with Cenobamate was evaluated after oral dosing in the mouse alternating current maximal electroshock seizure (AC-MES) assay (see Example 2).

The objective of this study was to characterize the dose-dependent anticonvulsant activity of Compound A and its pharmacological interaction with Cenobamate in the AC-MES assay in mice after a single oral administration of the compounds. The anticonvulsant efficacy of Compound A and Cenobamate, and the effect of combining Compound A with Cenobamate were tested in the AC-MES assay in male CF-1 mice following a single PO dose. Plasma and brain samples were obtained to understand the relationship between drug concentration and efficacy.

5.4.1 Materials and Method

The Compound A, vehicles, dose formulations, and test systems used in Example 4 was identical to those used in Example 2 substituting Lacosamide with Cenobamate.

Test Compound—Cenobamate

| | |
|---|---|
| Identity: | Cenobamate |
| Batch Number: | Batch 01 |
| Physical Description: | White powder |
| Purity: | 99.5% |
| Supplier: | Not available |

Experimental Design: The animals were assigned to treatment groups as indicated in Table 18 to Table 19. Four studies were conducted with Compound A and Cenobamate each. Their respective study dates are shown in Table 18 to Table 19.

TABLE 18
Experimental Groups - Study 4A

| | | | | | Pre- | | |
|---|---|---|---|---|---|---|---|
| | | | | | Treat- | | |
| | | | | | ment | Dose | |
| | | Dose | | Number | Time | Volume | Formu- |
| Group | Compound | (mg/kg) | Route | of Mice | (h) | (mL/kg) | lation |
| 1 | Vehicle | 0 | PO | 8 | 0.5 | 10 | F2 |
| 2 | Compound A | 1 | PO | 8 | 0.5 | 10 | F2 |
| 3 | Compound A | 5 | PO | 8 | 0.5 | 10 | F2 |
| 4 | Compound A | 10 | PO | 8 | 0.5 | 10 | F2 |

PO: per os, oral.

TABLE 19
Experimental Groups - Study 4B

| | | | | | Pre- | | |
|---|---|---|---|---|---|---|---|
| | | | | | Treat- | | |
| | | | | | ment | Dose | |
| | | Dose | | Number | Time | Volume | Formu- |
| Group | Compound | (mg/kg) | Route | of Mice | (h) | (mL/kg) | lation |
| 1 | Vehicle | 0 | PO | 8 | 0.5 | 10 | F2 |
| 2 | Compound A | 5 | PO | 8 | 0.5 | 10 | F2 |
| 3 | Compound A | 7.5 | PO | 7 | 0.5 | 10 | F2 |
| 4 | Compound A | 10 | PO | 9 | 0.5 | 10 | F2 |

PO: per os, oral.

TABLE 20
Experimental Groups - Study 4C

| | | | | | Pre- | | |
|---|---|---|---|---|---|---|---|
| | | | | | Treat- | | |
| | | | | | ment | Dose | |
| | | Dose | | Number | Time | Volume | Formu- |
| Group | Compound | (mg/kg) | Route | of Mice | (h) | (mL/kg) | lation |
| 1 | Vehicle | 0 | PO | 8 | 0.5 | 10 | F2 |
| 2 | Compound A | 3 | PO | 8 | 0.5 | 10 | F2 |

PO: per os, oral.

TABLE 21
Experimental Groups - Study 4F

| | | | | | Pre- | | |
|---|---|---|---|---|---|---|---|
| | | Dose | | Number | Treatment | Dose Volume | |
| Group | Compound | (mg/kg) | Route | of Mice | Time (h) | (mL/kg) | Formulation |
| 1 | Vehicle | 0 | PO | 8 | 2 | 10 | F2 + F1 |
| 2 | Compound A | 2 | PO | 8 | 0.5 | 10 | F2 + F1 |
| 3 | Cenobamate | 5 | PO | 8 | 2 | 10 | F2 + F1 |
| 4 | Compound A + | 2 + 5 | PO | 8 | 0.5 + 2 | 10 | F2 + F1 |
| | Cenobamate | | | | | | |

PO: per os, oral.

TABLE 22
Experimental Groups - Study 4D

| | | | | | Pre- | | |
|---|---|---|---|---|---|---|---|
| | | | | | Treat- | | |
| | | | | | ment | Dose | |
| | | Dose | | Number | Time | Volume | Formu- |
| Group | Compound | (mg/kg) | Route | of Mice | (h) | (mL/kg) | lation |
| 1 | Vehicle | 0 | PO | 8 | 2 | 10 | F1 |
| 2 | Cenobamate | 3 | PO | 8 | 2 | 10 | F1 |
| 3 | Cenobamate | 10 | PO | 8 | 2 | 10 | F1 |
| 4 | Cenobamate | 30 | PO | 8 | 8 | 10 | F1 |

PO: per os, oral.

TABLE 23
Experimental Groups - Study 4E

| | | | | | Pre- | | |
|---|---|---|---|---|---|---|---|
| | | | | | Treat- | | |
| | | | | | ment | Dose | |
| | | Dose | | Number | Time | Volume | Formu- |
| Group | Compound | (mg/kg) | Route | of Mice | (h) | (mL/kg) | lation |
| 1 | Vehicle | 0 | PO | 8 | 2 | 10 | F1 |
| 2 | Cenobamate | 3 | PO | 7 | 2 | 10 | F1 |
| 3 | Cenobamate | 5 | PO | 7 | 2 | 10 | F1 |
| 4 | Cenobamate | 7.5 | PO | 8 | 2 | 10 | F1 |

PO: per os, oral.

TABLE 24
Experimental Groups - Study 4G

| | | | | | Pre- | | |
|---|---|---|---|---|---|---|---|
| | | Dose | | Number | Treatment | Dose Volume | |
| Group | Compound | (mg/kg) | Route | of Mice | Time (h) | (mL/kg) | Formulation |
| 1 | Compound A + | 0.5 + 5 | PO | 8 | 0.5 +2 | 10 | F2 + F1 |
| | Cenobamate | | | | | | |
| 2 | Compound A + | 1 + 5 | PO | 8 | 0.5 +2 | 10 | F2 + F1 |
| | Cenobamate | | | | | | |

PO: per os, oral.

The Blinding and Randomization, Clinical Observations, AC-MES Assay, Sample Collection and Preparation, Plasma and Brain Samples Analysis, and Bioanalysis Procedures in Example 4 were identical to those used in Example 2.

Samples were analyzed for Compound A by a research-grade UHPLC-MS/MS method using the conditions listed below.

| Instrument: | Sciex TQ-5500 | |
|---|---|---|
| Matrix: | Mouse plasma (K₂EDTA) | |
| | Mouse brain homogenate | |
| Analyte: | Compound A | |
| Internal Standard (IS): | (S)-5-chloro-4-((1-(5-chloro-2-fluorophenyl)ethyl)amino)-2-fluoro-N- | |
| | (pyrazin-2-yl)benzenesulfonamide | |
| MS Conditions: | ESI: positive | |
| | Compound A: [M+H]⁺ m/z 369.2/247.2 | |
| | (S)-5-chloro-4-((1-(5-chloro-2-fluorophenyl)ethyl)amino)-2-fluoro-N- | |
| | (pyrazin-2-yl)benzenesulfonamide (IS): [M+H]⁺ m/z 458.793/157 | |
| UHPLC Conditions: | Time (min) | % B |
| | 0.0 | 10 |
| | 0.2 | 10 |
| | 3.4 | 6 |
| | 3.5 | 100 |
| | 4.0 | 100 |
| | 4.1 | 10 |
| | 5.0 | 10 |

| | |
|---|---|
| Calibration Standards | Mobile Phase A: 0.1% formic acid in water |
| | Mobile Phase B: 0.1% formic acid in acetonitrile |
| | Standards were prepared at 2.34, 4.69, 9.38, 18.8, 37.5, 75.0, 150, 300, |
| | 600, 1200, 2400, and 4800 ng/mL for 4A, 4B, and 4C. |
| | Standards were prepared at 0.586, 1.17, 2.34, 4.69, 9.38, 18.8, 37.5, |
| | 75.0, 150, 300, 600, and 1200 ng/mL for 4F. |
| | Standards were prepared at 0.293, 0.586, 1.17, 2.34, 4.69, 9.38, 18.8, |
| | 37.5, 75.0, 150, 300, 600 ng/mL for 4G. |
| QC Samples | QC samples were prepared at 14.0, 225 and 3600 ng/mL, analyzed in |
| | triplicate for 4A, 4B and 4C. |
| | QC samples were prepared at 3.5, 56.3 and 900 ng/mL, analyzed in |
| | triplicate for 4F. |
| | QC samples were prepared at 1.75, 28.1 and 450 ng/mL, analyzed in |
| | triplicate for 4G. |

Samples were analyzed for cenobamate by a research-grade UHPLC-MS/MS method using
the conditions listed below.

| Instrument: | Sciex TQ-5500 | |
|---|---|---|
| Matrix: | Mouse plasma (K₂EDTA) | |
| | Mouse brain homogenate | |
| Analyte: | Cenobamate | |
| Internal Standard (IS): | (2S,5R)-5-(4-((2-fluorobenzyl)oxy)phenyl)pyrrolidine-2-carboxamide | |
| | (4F, 4G); tert-butyl (2-(azetidin-l-ylmethyl)benzyl)(3,5-difluoro-4-(N- | |
| | (thiazol-4-yl)sulfamoyl)phenyl)carbamate (4D, 4E) | |
| MS Conditions: | ESI: positive | |
| | Cenobamate: [M+H]⁺ m/z 267.8/155.1 | |
| | (2S,5R)-5-(4-((2-fluorobenzyl)oxy)phenyl)pyrrolidine-2-carboxamide | |
| | (IS): [M+H]⁺ m/z 315.1/108.9 | |
| | tert-butyl (2-(azetidin-l-ylmethyl)benzyl)(3,5-difluoro-4-(A-(thiazol-4- | |
| | yl)sulfamoyl)phenyl)carbamate (IS): [M+H]⁺ m/z 551.1/214 | |
| UHPLC Conditions: | Time(min) | %B |
| | 0.0 | 20 |
| | 0.6 | 20 |
| | 1.00 | 100 |
| | 1.50 | 100 |
| | 1.60 | 20 |
| | 2.5 | 20 |

| | |
|---|---|
| | A: 0.1% formic acid in water |
| | B: 0.1% formic acid in acetonitrile |
| Calibration Standards | Standards were prepared at 2.34, 4.69, 9.38, 18.8, 37.5, 75.0, 150, 300, |
| | 600, 1200, 2400, and 4800 ng/mL for 4F, 4D, 4E, and 4G. |
| QC samples | QC samples were prepared at 14.0, 225 and 3600 ng/mL, analyzed in |
| | triplicate for 4F, 4D, 4E, and 4G. |

Sample concentration determination and Data Processing and Analysis in Example 4 was identical to Example 2.

5.4.2 Results

Bioanalysis: All system suitability test (SST), QC, matrix, and solvent blanks met acceptance criteria described in SOP MTD-066. The lower limit of quantitation (LLOQ) and upper limit of quantitation (ULOQ) for Compound A and Cenobamate are shown in Table 25.

TABLE 25
LLOQ and ULOQ for Compound A and Cenobamate

| Study | Compound | LLOQ | ULOQ |
|---|---|---|---|
| 4A | Compound A | 4.68 | 4800 |
| 4B | Compound A | 4.68 | 4800 |
| 4C | Compound A | 2.34 | 4800 |
| 4F | Compound A | 0.586 | 1200 |
| 4F | Cenobamate | 2.34 | 4800 |
| 4D | Cenobamate | 4.68 | 4800 |
| 4E | Cenobamate | 2.34 | 4800 |
| 4G | Compound A | 0.293 | 600 |
| 4G | Cenobamate | 2.34 | 4800 |

LLOQ: lower limit of quantitation.
ULOQ: upper limit of quantitation

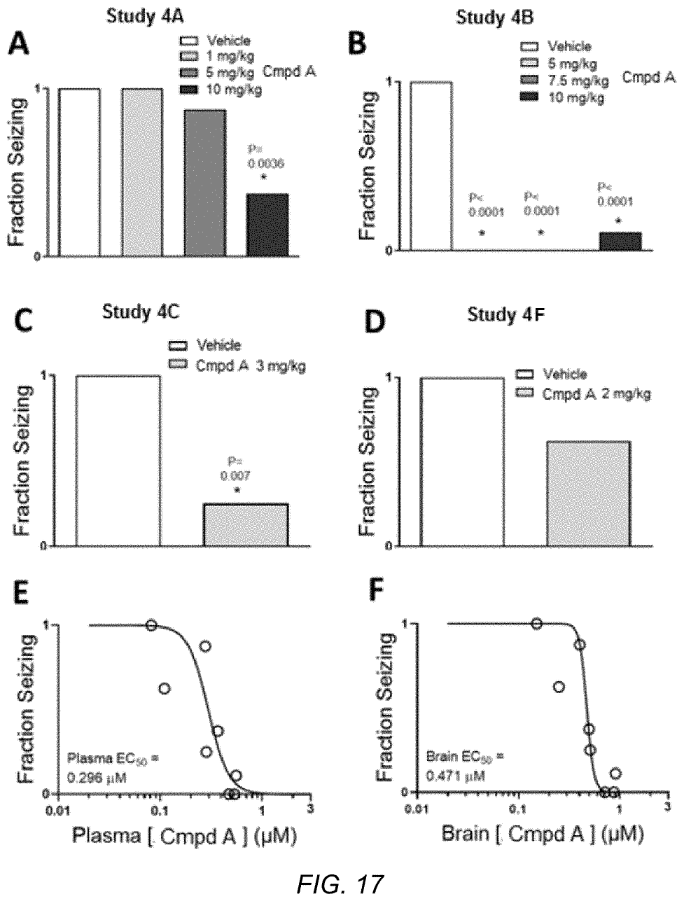

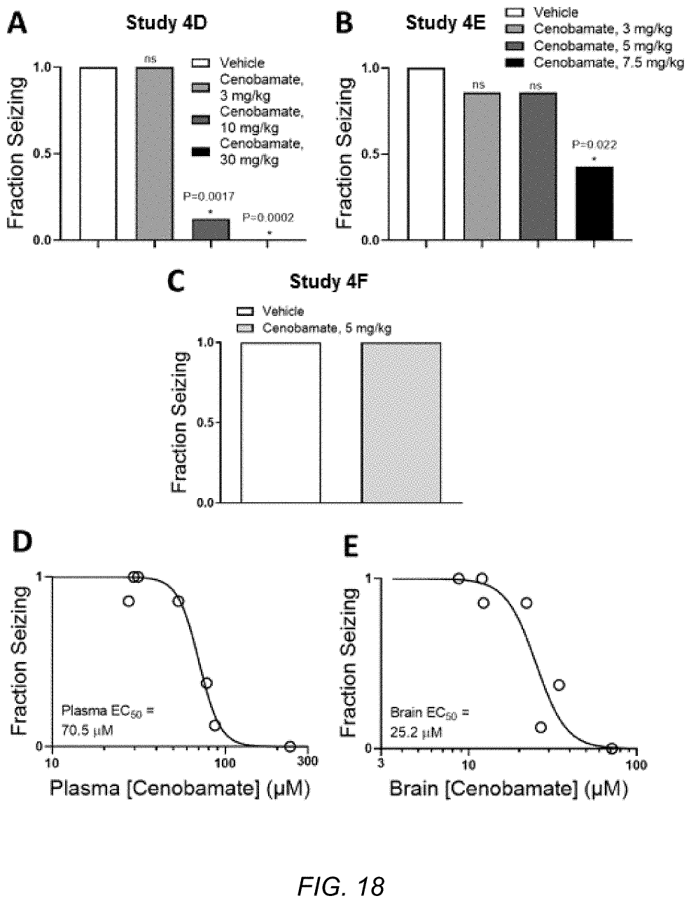

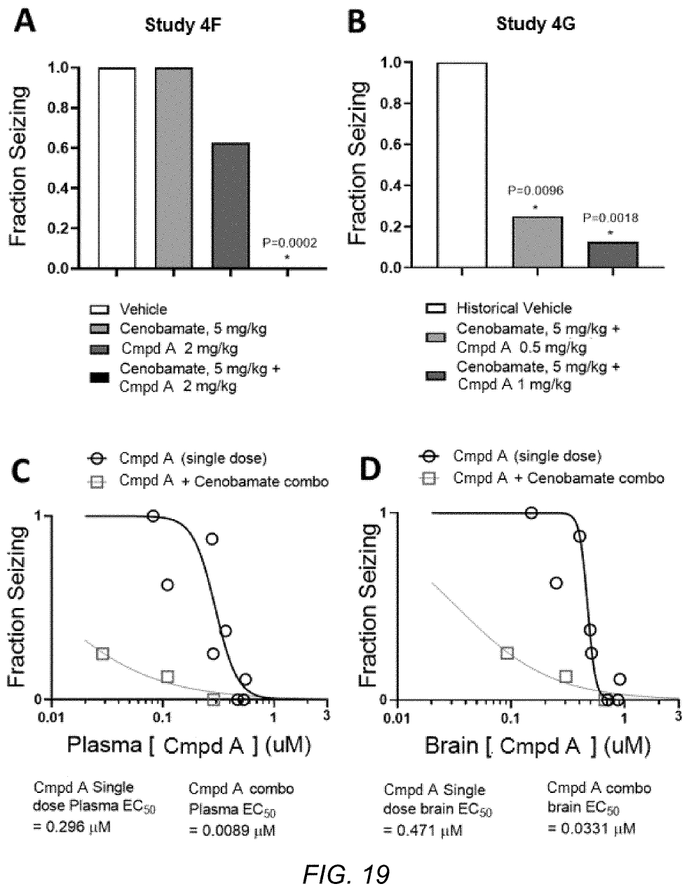

Efficacy in the AC-MES Assay: The results of the AC-MES-induced seizure study in mice are presented in Table 26 to Table 32, and in FIG. 17, FIG. 18, and FIG. 19.

Compound A Alone: Data from the dose and concentration responses of Compound A is summarized in FIG. 17, Table 26, Table 27 and Table 28. In all three efficacy studies (4A, 4B, 4C, and 4F), 100% of vehicle-treated CF-1 mice showed a tonic seizure with extension of hindlimbs. In Study 4A (at 1 mg/kg, n=8; 5 mg/kg, n=8; and 10 mg/kg, n=8), the fraction of animals that showed a tonic seizure response to the AC-MES stimulus following a 30 minute pre-treatment with Compound A decreased from 8/8 (mean plasma concentration of 0.08 μM; 1 mg/kg), to 7/8 (mean plasma concentration of 0.28 μM; 5 mg/kg), and to 3/8 (mean plasma concentration of 0.37 μM; 10 mg/kg). In Study 4B (5 mg/kg, n=8; 7.5 mg/kg, n=7 and 10 mg/kg, n=9), 5 mg/kg (mean plasma concentration of 0.47 μM) and 10 mg/kg (mean plasma concentration of 0.55 μM) doses of Compound A showed strong efficacy (0/8 and 1/9 animals seizing, respectively). In Study 4C (3 mg/kg, n=8), 3 mg/kg dose of Compound A showed 2/8 animals seizing at a plasma concentration of 0.28 μM. In study 4F (2 mg/kg, n=8), 2 mg/kg dose showed 5/8 animals seizing at a plasma concentration of 0.11 μM. In all efficacy studies except 4F, the difference between Compound A-treated groups and vehicle-dosed groups reached statistical significance (p-values are shown in FIG. 17).

One mouse from the 10 mg/kg group in Study 4A and three mice in study 4B (two mice from the 10 mg/kg group and one mouse from the 7.5 mg/kg group) showed behavioral signs (tremors, decreased activity, and splayed hindlimbs) (plasma concentrations: 4A: 0.39 μM; 4B: 10 mg/kg: 0.61 and 0.52 μM, 7.5 mg/kg: 0.75 μM).

The composite concentration response curve of Compound A from the three studies showed an EC₅₀ for plasma of 0.30 μM and an EC₅₀ for brain tissue of 0.47 μM (FIG. 17). The concentrations of Compound A reached in brain tissue and plasma were dose-linear (FIG. 17).

Cenobamate Alone: Prior to testing of Compound A in combination with Cenobamate in the AC-MES model (Study 4F and 4G), we tested cenobamate alone at different doses to establish a full dose-response. Data from the dose and concentration response of Cenobamate is summarized in FIG. 18, Table 29, and Table 30. Seven PO doses of Cenobamate were each tested in the AC-MES model separately (Study 4D: 3 mg/kg, 10 mg/kg, and 30 mg/kg, n=8 per group; Study 4E: 3 mg/kg, 5 mg/kg, and 7.5 mg/kg, n=8; and Study 4F: 5 mg/kg, n=8) and as part of the studies. Two hours after administration of Cenobamate at 3 mg/kg (n=15), 5 mg/kg (n=7), 7.5 mg/kg (n=7), 10 mg/kg (n=8), and 30 mg/kg (n=8) via oral gavage to male CF-1 mice, the mice underwent an AC-MES stimulus. Cenobamate showed dose- and concentration-dependent effect against AC-MES-induced tonic seizures with 3/7 animals seizing at 7.5 mg/kg (mean plasma concentration of 78.1 μM), 1/8 animals seizing at 10 mg/kg (mean plasma concentration of 87 μM), 0/8 animals seizing at 30 mg/kg (mean plasma concentration of 24 μM), and minimal effect at lower doses of 3 and 5 mg/kg. Data from all 3 studies (4D, 4E, and 4F) were combined for concentration response curve analysis of Cenobamate when dosed alone (FIG. 18). The concentration response curve of Cenobamate showed an EC₅₀ for plasma of 70.5 μM and an EC₅₀ for brain tissue of 25.2 μM.

Combination of Compound A and Cenobamate: Based on dose-response studies, a dose of 5 mg/kg cenobamate was chosen and doses of 0.5, 1, and 2 mg/kg Compound A that showed minimal efficacy when dosed alone for the combination study in the AC-MES model.

Data from the combination studies is summarized in FIG. 19, Table 31, and Table 32. We performed two combination studies, 4F (Compound A at 2 mg/kg combined with cenobamate at 5 mg/kg) and 4G (Compound A at 0.5 and 1 mg/kg combined with cenobamate at 5 mg/kg). Combining Compound A (0.5, 1, and 2 mg/kg PO, 0.5 hours before AC-MES) with Cenobamate (5 mg/kg PO, 2 hours before AC-MES) in male CF-1 mice showed 2/8 animals seizing at 0.5 mg/kg Compound A, 1/8 animals seizing at 1 mg/kg Compound A, and 0/8 animals seizing at 2 mg/kg Compound A compared to partial or no effect when Compound A (5/8 animals seizing when Compound A dosed alone at 2 mg/kg) or Cenobamate (8/8 animals seizing when cenobamate dosed alone at 5 mg/kg) were dosed alone. In study 4F, plasma concentrations of Compound A (mean plasma concentration at 2 mg/kg: 0.11 μM when dosed alone and 0.29 when dosed in combination) and Cenobamate (mean plasma concentration at 5 mg/kg: 31.3 μM when dosed alone and 38.9 μM when dosed in combination) were slightly higher when dosed in combination compared to when dosed alone. In study 4G, mean total plasma concentrations achieved were: Compound A dosed in combination at 0.5 mg/kg: 0.03 μM; Compound A dosed in combination at 1 mg/kg: 0.11 μM; Cenobamate dosed in combination at 5 mg/kg (+0.5 mg/kg Compound A): 41.1 μM; cenobamate dosed in combination at 5 mg/kg (+1 mg/kg Compound A): 39.7 μM. The concentration response curve of Compound A and Cenobamate dosed in combination showed an extrapolated EC₅₀ for plasma of 0.01 μM and for brain tissue of 0.03 μM for Compound A.

TABLE 26
Study 4A: Summary of the Anticonvulsant Effects of Compound A
in the Mouse AC-MES Following a Single Oral Dose

| Compound ID | Plasma | Brain | B/P | Fraction | Behavioral |
|---|---|---|---|---|---|
| and Dose | (μM) | (μM) | Ratio | Seizing | Signs |
| Vehicle | n/a | n/a | n/a | 8/8 | 0/8 |
| Compound A (1 mg/kg) | 0.08 | 0.15 | 1.85 | 8/8 | 0/8 |
| Compound A (5 mg/kg) | 0.28 | 0.40 | 1.45 | 7/8 | 0/8 |
| Compound A (10 mg/kg) | 0.37 | 0.50 | 1.36 | 3/8 | 1/8 |

B/P: brain to plasma ratio; n/a: not applicable.

TABLE 27
Study 4B: Summary of the Anticonvulsant Effects of Compound A
in the Mouse AC-MES Following a Single Oral Dose

| | Mean | Mean | | | Fraction with |
|---|---|---|---|---|---|
| Compound ID | Plasma | Brain | B/P | Fraction | Behavioral |
| and Dose | (μM) | (μM) | Ratio | Seizing | Signs |
| Vehicle | n/a | n/a | n/a | 8/8 | 0/8 |
| Compound A (5 mg/kg) | 1 | 0.71 | 1.51 | 0/8 | 0/8 |
| Compound A (7.5 mg/kg) | 0.53 | 0.87 | 1.64 | 0/7 | 1/7 |
| Compound A (10 mg/kg) | 0.55 | 0.91 | 1.64 | 1/9 | 2/9 |

B/P: brain to plasma ratio; n/a: not applicable.

TABLE 28
Study 4C: Summary of the Anticonvulsant Effects of Compound A
in the Mouse AC-MES Following a Single Oral Dose

| | Mean | Mean | | | Fraction with |
|---|---|---|---|---|---|
| Compound ID | Plasma | Brain | B/P | Fraction | Behavioral |
| and Dose | (μM) | (μM) | Ratio | Seizing | Signs |
| Vehicle | n/a | n/a | n/a | 8/8 | 0/8 |
| Compound A (3 mg/kg) | 0.28 | 0.51 | 1.79 | 2/8 | 0/8 |

B/P: brain to plasma ratio; n/a: not applicable.

TABLE 29
Study 4D: Summary of the Anticonvulsant Effects of Cenobamate
in the Mouse AC-MES Following a Single Oral Dose

| | Mean | Mean | | | Fraction with |
|---|---|---|---|---|---|
| Compound ID | Plasma | Brain | B/P | Fraction | Behavioral |
| and Dose | (μM) | (μM) | Ratio | Seizing | Signs |
| Vehicle | n/a | n/a | n/a | 8/8 | 0/8 |
| Cenobamate (3 mg/kg) | 29.6 | 8.70 | 0.29 | 8/8 | 0/8 |
| Cenobamate (10 mg/kg) | 87.0 | 26.8 | 0.31 | 1/8 | 0/8 |
| Cenobamate (30 mg/kg) | 237 | 71.3 | 0.30 | 0/8 | 0/8 |

B/P: brain to plasma ratio; n/a: not applicable.

TABLE 30
Study 4E: Summary of the Anticonvulsant Effects of Cenobamate
in the Mouse AC-MES Following a Single Oral Dose

| | Mean | Mean | | | Fraction with |
|---|---|---|---|---|---|
| Compound ID | Plasma | Brain | B/P | Fraction | Behavioral |
| and Dose | (μM) | (μM) | Ratio | Seizing | Signs |
| Vehicle | n/a | n/a | n/a | 8/8 | 0/8 |
| Cenobamate (3 mg/kg) | 27.7 | 12.2 | 0.44 | 6/7 | 0/8 |
| Cenobamate (5 mg/kg) | 53.6 | 22.1 | 0.41 | 6/7 | 0/8 |
| Cenobamate (7.5 mg/kg) | 78.1 | 34.5 | 0.44 | 3/8 | 0/8 |

B/P: brain to plasma ratio; n/a: not applicable.

TABLE 31
Study 4F: Summary of the Anticonvulsant Effects of Compound A in
Combination with Cenobamate in the Mouse AC-MES
Following a Single Oral Dose

| | | | | | Fraction |
|---|---|---|---|---|---|
| | Mean | Mean | | | with |
| Compound ID | Plasma | Brain | B/P | Fraction | Behavioral |
| and Dose | (μM) | (μM) | Ratio | Seizing | Signs |
| Vehicle | n/a | n/a | n/a | 8/8 | 0/8 |
| Compound A | 0.11 | 0.25 | 2.32 | 5/8 | 0/8 |
| (2 mg/kg) | | | | | |
| Cenobamate | 31.3 | 12.0 | 0.38 | 8/8 | 0/8 |
| (5 mg/kg) | | | | | |
| Compound A + | 0.29 + 38.9 | 0.67 + 14.7 | 2.34 + 0.38 | 0/8 | 0/8 |
| Cenobamate | | | | | |
| (2+5) | | | | | |

B/P: brain to plasma ratio; n/a: not applicable.

TABLE 32
Study 4G: Summary of the Anticonvulsant Effects of
Compound A in Combination with
Cenobamate in the Mouse AC-MES Following a Single Oral Dose

| | | | | | Fraction |
|---|---|---|---|---|---|
| | Mean | Mean | | | with |
| Compound ID | Plasma | Brain | B/P | Fraction | Behavioral |
| and Dose | (μM) | (μM) | Ratio | Seizing | Signs |
| Compound A + | 0.03 + 41.1 | 0.09 + 18.1 | 3.22 + 0.44 | 2/8 | 0/8 |
| Cenobamate | | | | | |
| (0.5 mg/kg + | | | | | |
| 5 mg/kg) | | | | | |
| Compound A + | 0.11 + 39.7 | 0.30 + 17.0 | 2.76 + 0.43 | 1/8 | 0/8 |
| Cenobamate | | | | | |
| (1 mg/kg + | | | | | |
| 5 mg/kg) | | | | | |

5.4.3 Conclusions

Compound A and Cenobamate demonstrated concentration-dependent efficacy in the CF-1 mouse AC-MES assay. Plasma and brain Compound A EC₅₀ values projected from the concentration-response curve analysis were 0.30 μM and 0.47 μM, respectively. The composite concentration response curve of Compound A and Cenobamate dosed in combination projected an EC₅₀ for plasma of 0.01 μM and for brain tissue of 0.03 μM for Compound A. The results project an increase in the potency of Compound A by 33.3 fold and 14.2 fold for plasma and brain tissue, respectively when dosed in combination with Cenobamate.

Six oral (PO) doses of Compound A were each tested in the AC-MES assay. Thirty minutes after administration of Compound A at 1 mg/kg (n=8), 2 mg/kg (n=8), 3 mg/kg (n=8), 5 mg/kg (n=16), 7.5 mg/kg (n=7), and 10 mg/kg (n=17) via oral gavage to male CF-1 mice, the mice underwent a 60 Hz corneal electrical stimulus (0.2 seconds duration, 40 mA). This stimulus elicited a tonic hindlimb extension in all vehicle-dosed animals. Any animal that did not show hindlimb extension in response to the electrical stimulus was considered protected. An observational neurological assessment (qualitative test) was also conducted at the time of testing as a tolerability screen. Terminal plasma and brain samples were collected from all animals to obtain the levels of Compound A and cenobamate concentrations, and to understand the relationship between efficacy and drug concentration.

After a single oral dose, Compound A showed concentration-dependent effect against AC-MES-induced tonic seizures with maximal effect of 0/8 animals seizing at a plasma concentration of 0.470 μM (from Study 4B at 5 mg/kg). In Study 4A (at 1 mg/kg, n=8; 5 mg/kg, n=8; and 10 mg/kg, n=8), the fraction of animals that showed a tonic seizure response to the AC-MES stimulus following a 30 minute pre-treatment with Compound A decreased from 8/8 (mean plasma concentration of 0.08 μM; 1 mg/kg), to 7/8 (mean plasma concentration of 0.28 μM; 5 mg/kg), and to 3/8 (mean plasma concentration of 0.37 μM; 10 mg/kg). In Study 4B (5 mg/kg, n=8; 7.5 mg/kg, n=7 and 10 mg/kg, n=9), 5 mg/kg (mean plasma concentration of 0.47 μM) and 10 mg/kg (mean plasma concentration of 0.55 μM) doses of Compound A showed strong efficacy (0/8 and 1/9 animals seizing, respectively). In Study 4C (3 mg/kg, n=8), 3 mg/kg dose of Compound A showed 2/8 animals seizing at a plasma concentration of 0.28 μM. In study 4F (2 mg/kg, n=8), 2 mg/kg dose showed 5/8 animals seizing at a plasma concentration of 0.11 μM. In all efficacy studies except 4F, the difference between Compound A-treated groups and vehicle-dosed groups reached statistical significance (p values shown in FIG. 17). Data from all four studies were combined for concentration-response curve analysis of Compound A when dosed alone. The concentration-response curve of Compound A showed a half-maximum effective concentration (EC₅₀) for plasma of 0.30 μM and an EC₅₀ for brain tissue of 0.47 μM.

One mouse from the 10 mg/kg group in Study 4A and three mice in study 4B (two mice from the 10 mg/kg group and one mouse from the 7.5 mg/kg group) showed behavioral signs (tremors, decreased activity, and splayed hindlimbs) (plasma concentrations: 4A: 0.39 μM; 4B: 10 mg/kg: 0.61 and 0.52 μM, 7.5 mg/kg: 0.75 μM).

Prior to testing of Compound A in combination with cenobamate in the AC-MES model, Cenobamate was test alone at different doses to establish a full dose-response. Seven PO doses of Cenobamate were each tested in the AC-MES model separately (Study 4D: 3 mg/kg, 10 mg/kg, and 30 mg/kg, n=8 per group; Study 4E: 3 mg/kg, 5 mg/kg, and 7.5 mg/kg, n=8; and Study 4F: 5 mg/kg, n=8) and as part of the studies. Two hours after administration of cenobamate at 3 mg/kg (n=15), 5 mg/kg (n=7), 7.5 mg/kg (n=7), 10 mg/kg (n=8), and 30 mg/kg (n=8) via oral gavage to male CF-1 mice, the mice underwent an AC-MES stimulus. Cenobamate showed dose- and concentration-dependent efficacy against AC-MES-induced tonic seizures with 3/7 animals seizing at 7.5 mg/kg (mean plasma concentration of 78.1 μM), 1/8 animals seizing at 10 mg/kg (mean plasma concentration of 87 μM), 0/8 animals seizing at 30 mg/kg (mean plasma concentration of 237 μM). Lower doses of 3 and 5 mg/kg had minimal effect with 7/8 animals seizing. The fraction of animals seizing in the Cenobamate-treated group was significantly different in 2/3 studies from the vehicle-treated group (p-values shown in FIG. 18). Data from all 3 studies (4D, 4E, and 4F) were combined for concentration response curve analysis of Cenobamate when dosed alone. The concentration response curve of Cenobamate showed an EC₅₀ for plasma of 70.5 μM and an EC₅₀ for brain tissue of 25.2 μM.

Based on dose-response studies, a dose of 5 mg/kg Cenobamate and 0.5, 1, and 2 mg/kg Compound A was chosen that showed minimal efficacy when dosed alone for the combination study in the AC-MES model.

Two combination studies were performed, 4F (Compound A at 2 mg/kg combined with Cenobamate at 5 mg/kg) and 4G (Compound A at 0.5 and 1 mg/kg combined with Cenobamate at 5 mg/kg). Combining Compound A (0.5, 1, and 2 mg/kg PO, 0.5 hours before AC-MES) with Cenobamate (5 mg/kg PO, 2 hours before AC-MES) in male CF-1 mice showed 2/8 animals seizing at 0.5 mg/kg Compound A, 1/8 animals seizing at 1 mg/kg Compound A, and 0/8 animals seizing at 2 mg/kg Compound A compared to 5/8 animals seizing when Compound A dosed alone at 2 mg/kg or 8/8 animals seizing when Cenobamate dosed alone at 5 mg/kg. In study 4F, the difference between the Cenobamate-treated group and the combination group was statistically significant, whereas the difference between the Compound A-treated group and combination group was not statistically significant. In study 4G, the difference between the vehicle group and the combination groups was statistically significant (note: vehicle control group and single dose control groups of Compound A and Cenobamate were not used in this study; p-values are shown in FIG. 19). In study 4F, plasma concentrations of Compound A (mean plasma concentration at 2 mg/kg: 0.11 μM when dosed alone and 0.29 when dosed in combination) and Cenobamate (mean plasma concentration at 5 mg/kg: 31.3 μM when dosed alone and 38.9 μM when dosed in combination) were slightly higher when dosed in combination compared to when dosed alone. In study 4G, mean total plasma concentrations achieved were: Compound A when dosed combination at 0.5 mg/kg: 0.03 μM; Compound A when dosed in combination at 1 mg/kg: 0.11 μM; Cenobamate when dosed combination at 5 mg/kg (0.5 mg/kg Compound A): 41.1 μM; Cenobamate when dosed in combination at 5 mg/kg (1 mg/kg Compound A): 39.7 μM.

The composite concentration response curve of Compound A and Cenobamate dosed in combination showed an extrapolated EC₅₀ of Compound A for plasma of 0.01 μM and for brain tissue of 0.03 μM. The results show an increase in the potency of Compound A by 33.3 fold and 14.2 fold for plasma and brain tissue, respectively when dosed in combination with Cenobamate.

All of the U.S. patents, U.S. patent application publications, U.S. patent applications, foreign patents, foreign patent applications, and non-patent publications referred to in this specification, including U.S. non-provisional appl. No. 63/147,736, filed Feb. 9, 2021, are incorporated herein by reference in their entireties.

Although the foregoing compositions, methods, and uses have been described in some detail to facilitate understanding, it will be apparent that certain changes and modifications may be practiced within the scope of the appended claims. Accordingly, the described embodiments are to be considered as illustrative and not restrictive, and the claimed invention is not to be limited to the details given herein, but may be modified within the scope and equivalents of the appended claims.

1. A method of treating a seizure disorder in a human in need thereof, comprising conjointly administering Compound A and an antiseizure medication (ASM) to the human in amounts that are therapeutically effective when conjointly administered;
wherein Compound A is N-[4-(6-fluoro-3,4-dihydro-1H-isoquinolin-2-yl)-2,6-dimethylphenyl]-3,3-dimethylbutanamide.

2. A method of reducing the amount of an antiseizure medication (ASM) that is required for therapeutic efficacy in a human suffering from a seizure disorder, comprising administering to the human, conjointly with the ASM, an amount of Compound A that is effective to achieve such reduction when administered with the ASM;
wherein Compound A is N-[4-(6-fluoro-3,4-dihydro-1H-isoquinolin-2-yl)-2,6-dimethylphenyl]-3,3-dimethylbutanamide.

3. A method of reducing the amount of Compound A that is required for therapeutic efficacy in a human suffering from a seizure disorder, comprising administering to the human, conjointly with Compound A, an amount of an antiseizure medication (ASM) that is effective to achieve such reduction when administered with Compound A;
wherein Compound A is N-[4-(6-fluoro-3,4-dihydro-1H-isoquinolin-2-yl)-2,6-dimethylphenyl]-3,3-dimethylbutanamide.

4. The method of any one of claims 1-3, which comprises enhancing the opening of a Kv7 potassium channel in the human.

5. A method of enhancing the opening of a Kv7 potassium channel in a human, comprising conjointly administering Compound A and an antiseizure medication (ASM) to the human in amounts that are therapeutically effective when conjointly administered;
wherein compound A is N-[4-(6-fluoro-3,4-dihydro-1H-isoquinolin-2-yl)-2,6-dimethylphenyl]-3,3-dimethylbutanamide; and
wherein the human has a seizure disorder.

6. The method of claim 1, which comprises enhancing the opening of a Kv7 potassium channel in the human, wherein the Kv7 potassium channel is one or more of Kv7.2, Kv7.3, Kv7.4, or Kv7.5.

7. The method of claim 6, which is selective for enhancing the opening of one or more of Kv7.2, Kv7.3, Kv7.4, or Kv7.5 over Kv7.1.

8. The method of claim 1, which comprises opening of the Kv7.2/Kv7.3 (KCNQ2/3) potassium channel.

9. The method of claim 1, wherein the ASM is a benzodiazepine, carbamazepine, cenobamate, felbamate, gabapentin, lacosamide, lamotrigine, levetiracetam, oxcarbazepine, phenobarbital, phenytoin, pregabalin, rufinamide, tiagabine, topiramate, valproic acid, vigabatrin, zonisamide, or a combination thereof.

10. The method of claim 1, wherein the ASM is valproic acid, levetiracetam, phenytoin, lacosamide, or cenobamate.

11. The method of claim 1, wherein the ASM does not enhance the opening of a Kv7 potassium channel in the human.

12. The method of claim 1, wherein the ASM decreases neuronal excitation in the human.

13. The method of claim 12, wherein the ASM decreases neuronal excitation by blocking a sodium channel in the human.

14. The method of claim 12, wherein the ASM decreases neuronal excitation by blocking a calcium channel in the human.

15. The method of claim 12, wherein the ASM decreases neuronal excitation by binding to synaptic vesicle glycoprotein 2A (SV2A) in the human.

16. The method of claim 1, wherein the ASM increases neuronal inhibition in the human.

17. The method of claim 1, wherein the ASM is a glutamatergic agent.

18. The method of claim 1, wherein the ASM is a GABAergic agent.

19. The method of claim 1, wherein the seizure disorder is associated with Kv7 potassium channel dysfunction.

20. The method of claim 1, wherein the seizure disorder is focal onset epilepsy.

21. The method of claim 1, wherein Compound A is orally administered to the human.

22. The method of claim 1, wherein the ASM is orally administered to the human.

23. The method of claim 1, wherein Compound A is administered at a dose of 1 to 200 mg to the human.

24. The method of claim 1, wherein Compound A is administered at a dose of 2 to 100 mg to the human.

25. The method of claim 1, wherein Compound A is administered at a dose of 5 to 50 mg to the human.

26. The method of claim 1, wherein Compound A is administered at a dose of 5, 10, 15, 20, or 25 mg to the human.

27. The method of claim 1, wherein Compound A is administered at a dose of 20 mg to the human.

28. The method of claim 1, wherein Compound A is administered at a dose of at least 10 mg to the human.

29. The method of claim 28, wherein Compound A is administered at a dose of at least 20 mg to the human.

30. (canceled)

31. The method of claim 1, wherein Compound A is administered at a dose of 5-1000 mg per day to the human.

32. (canceled)

33. (canceled)

34. The method of claim 31, wherein Compound A is administered at a dose of 20-150 mg per day to the human.

35. (canceled)

36. The method of claim 1, wherein Compound A is administered at a dose of 0.01-2.0 mg/kg to the human.

37. The method of claim 36, wherein Compound A is administered at a dose of 0.03-1.0 mg/kg to the human.

38. The method of claim 36, wherein Compound A is administered at a dose of 0.05-0.5 mg/kg to the human.

39. The method of claim 1, wherein Compound A is orally administered to the human from between about 30 minutes before to about 2 hours after eating a meal.

40. The method of claim 39, wherein Compound A is orally administered to the human during a meal or within 15 minutes after eating a meal.

41. The method of claim 1, wherein the ASM is valproic acid.

42. The method of claim 41, wherein the valproic acid is administered at a dose of 2-16 mg/kg to the human.

43. The method of claim 41, wherein the valproic acid is administered at a dose of 4-12 mg/kg to the human.

44. The method of claim 1, wherein the ASM is phenytoin.

45. The method of claim 44, wherein the phenytoin is administered at a dose of 0.05-5 mg/kg to the human.

46. The method of claim 44, wherein the phenytoin is administered at a dose of 0.1-1 mg/kg to the human.

47. The method of claim 1, wherein the ASM is lacosamide.

48. The method of claim 47, wherein the lacosamide is administered at a dose of 0.1-5 mg/kg to the human.

49. The method of claim 47, wherein the lacosamide is administered at a dose of 0.5-1 mg/kg to the human.

50. The method of claim 1, wherein the ASM is cenobamate.

51. The method of claim 50, wherein the cenobamate is administered at a dose of 0.05-5 mg/kg to the human.

52. The method of claim 50, wherein the cenobamate is administered at a dose of 0.1-1 mg/kg to the human.

53. The method of claim 1, wherein the conjoint administration of Compound A and the ASM provides improved efficacy relative to individual administration of Compound A or the ASM alone.

54. A pharmaceutical composition comprising Compound A, an antiseizure medication (ASM), and a pharmaceutically acceptable carrier;
wherein Compound A is N-[4-(6-fluoro-3,4-dihydro-1H-isoquinolin-2-yl)-2,6-dimethylphenyl]-3,3-dimethylbutanamide.

55. The pharmaceutical composition of claim 54, wherein the ASM is a benzodiazepine, carbamazepine, cenobamate, felbamate, gabapentin, lacosamide, lamotrigine, levetiracetam, oxcarbazepine, phenobarbital, phenytoin, pregabalin, rufinamide, tiagabine, topiramate, valproic acid, vigabatrin, zonisamide, or a combination thereof.

56. The pharmaceutical composition of claim 55, wherein the ASM is valproic acid, phenytoin, levetiracetam, lacosamide, or cenobamate.

57. The pharmaceutical composition of claim 56, wherein the ASM is valproic acid.

58. The pharmaceutical composition of claim 56, wherein the ASM is phenytoin.

59. The pharmaceutical composition of claim 56, wherein the ASM is levetiracetam.

60. The pharmaceutical composition of claim 56, wherein the ASM is lacosamide.

61. The pharmaceutical composition of claim 56, wherein the ASM is cenobamate.
